(19) United States
(12) Patent Application Publication
(10) Pub. No.: US 2025/0179043 A1
(43) Pub. Date: Jun. 5, 2025

(54) PROCESSES FOR THE PREPARATION OF N-(1-METHYLCYCLOPROPYL)-2-(3-PYRIDINYL)-2H-INDAZOLE-4-CARBOXAMIDE AND INTERMEDIATES THEREOF

(71) Applicant: FMC CORPORATION, Philadelphia, PA (US)

(72) Inventors: Ahmad EL-AWA, Wilmington, DE (US); Linli HE, West Chester, PA (US); Junbae HONG, Newark, DE (US); Jessica L. KLINKENBERG, Wilmington, DE (US); William Eugene PALMER, Shadyside, OH (US); Mahesh PATIL, Ankleshwar (IN); Prabhakar PEDDIKOTLA, Hyderabad (IN); Xianmiao QIAN, Shanghai (CN); Hao WANG, Shanghai (CN); Ning XU, Shanghai (CN); Jonathan KEIM, Newark, DE (US); R. Paul MANCHESTER, Newark, DE (US); Prafull SAWARKAR, Panoli (IN); Vipul DUDHAT, Panoli (IN)

(21) Appl. No.: 18/839,283

(22) PCT Filed: Feb. 17, 2023
(86) PCT No.: PCT/US2023/013267

§ 371 Date: Aug. 16, 2024

Related U.S. Application Data
(60) Provisional application No. 63/311,276, filed on Feb. 17, 2022.

(51) Int. Cl. C07D 401/04

(52) U.S. Cl. CPC C07D 401/04

(57) ABSTRACT

Disclosed are processes for the preparation of the pesticide N-(1-methylcyclopropyl)-2-(3-pyridinyl)-2H-indazole 4-carboxamide. Also disclosed are methods for the preparation of intermediates of thereof N-(1-methylcyclopropyl)-2-(3-pyridinyl)-2H-indazole 4-carboxamide.

### CROSS-REFERENCE TO RELATED APPLICATION

This application claims the benefit of U.S. Provisional Application No. 63/311,276 filed Feb. 17, 2022.

### FIELD OF THE DISCLOSURE

The field of the disclosure relates generally to processes for the preparation of N-(1-methylcyclopropyl)-2-(3-pyridinyl)-2H-indazole 4-carboxamide and intermediates thereof.

### BACKGROUND

International Publication Number WO 2015/038503 discloses useful pesticidal indazole compounds and processes of preparation thereof. N-(1-methylcyclopropyl)-2-(3-pyridinyl)-2H-indazole 4-carboxamide corresponding to CAS registry no. 1689545-27-4 is among the indazole compounds disclosed.

A need exists for improved processes for the preparation of N-(1-methylcyclopropyl)-2-(3-pyridinyl)-2H-indazole 4-carboxamide and associated intermediate compounds.

### BRIEF DESCRIPTION

One aspect of the disclosure is directed to a process for the preparation of compound 775. The process comprising forming a reaction mixture comprising compound 223, CH₃Cl, an alkali metal iodide, a base, and a solvent system, and reacting the reaction mixture to form a reaction product mixture comprising compound 775 according to the following scheme

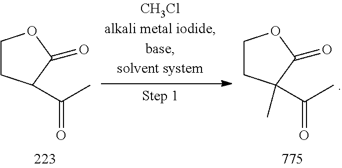

Another aspect of the disclosure is directed to a process for the preparation of compound 200 according to a first scheme, a second scheme, or a third scheme.

The first such scheme for preparing compound 200 comprises steps 1 to 3.

Step 1 comprises forming a reaction mixture comprising compound 775, HCl, and a solvent system, and reacting the reaction mixture to form a reaction product mixture comprising compound 069 according to the following reaction scheme

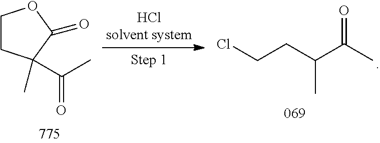

Step 2 comprises forming a reaction mixture comprising compound 069, a base, and a solvent system, and reacting the reaction mixture to form a reaction product mixture comprising compound 079 according to the following reaction scheme

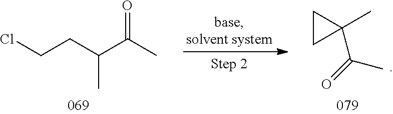

Step 3 comprises forming a reaction mixture comprising compound 079, an oxidizing agent, optionally a base, and a solvent system, and reacting the reaction mixture to form a reaction product mixture comprising compound 200 according to the following reaction scheme

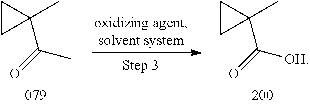

The second such scheme for preparing compound 200 comprises steps 1 to 2.

Step 1 comprises forming a reaction mixture comprising compound 900, CHX₃, a base, a solvent system, and a phase transfer catalyst, and reacting the reaction mixture to form a reaction product mixture comprising compound 905 according to the following reaction scheme

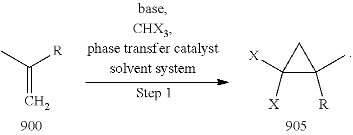

wherein R is selected from COOCH₃, COOCH₂CH₃, COOH, and CN, and wherein each X is independently selected from Cl, Br, and I.

When R is COOCH₃, COOCH₂CH₃, or CN, the process further comprises Step 1b forming a reaction mixture comprising compound 905, a base, and a solvent system, and reacting the reaction mixture to form a reaction product mixture comprising compound 906 according to the following reaction scheme

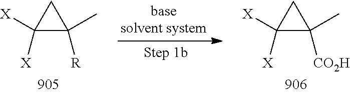

Step 2 comprises forming a reaction mixture comprising compound 906, a reducing agent, and a solvent system, and reacting the reaction mixture to form a reaction product mixture comprising compound 200 according to the following reaction scheme

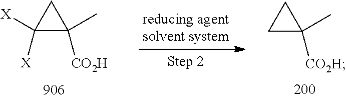

When R is COOH, the process comprises Step 2′, Step 2′ comprising forming a reaction mixture comprising compound 905, a reducing agent, and a solvent system, and reacting the reaction mixture to form a reaction product mixture comprising compound 200 according to the following scheme

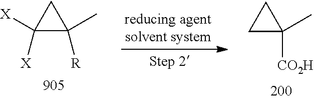

The third such scheme for preparing compound 200 comprises steps 1 and 2.

Step 1 comprises forming a reaction mixture comprising acetic acid, compound 100, a base, a photocatalyst, and a solvent system, and reacting the reaction mixture by exposure to light emitted from at least one light source to form a reaction product mixture comprising compound 110 according to the following reaction scheme

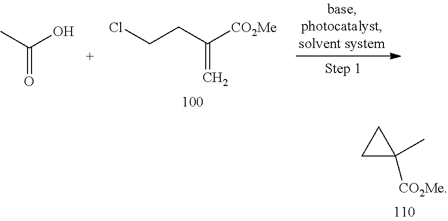

Step 2 comprises forming a reaction mixture comprising compound 110, a solvent system, and a base, and reacting the reaction mixture to form a reaction product mixture comprising compound 200 according to the following

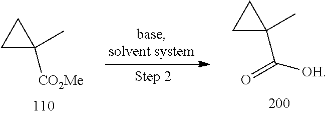

Another aspect of the disclosure is directed to a process for preparing compound 070, the process comprising a first scheme and a second scheme.

The first scheme comprising two steps:

Step 1 comprising substep (a) comprising forming a reaction mixture comprising compound 200, a chlorinating reagent, optionally a catalyst, and a solvent system and reacting the reaction mixture to form a reaction product mixture comprising an acid chloride intermediate, followed by substep (b) comprising forming a reaction mixture by combining the reaction product mixture from substep (a) with an ammonia source and reacting the reaction mixture to form a reaction product mixture comprising compound 144 according to the following reaction scheme

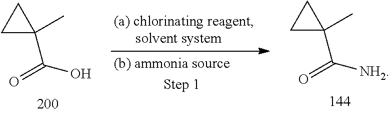

Step 2 comprises substep (a) comprising forming a reaction mixture comprising compound 144, a base, an oxidant, and a solvent system, and reacting the reaction mixture to form a reaction product mixture comprising a N-halocarboxamide intermediate, followed by substep (b) comprising heating the reaction product mixture comprising the N-halocarboxamide intermediate to form a reaction product mixture comprising compound 070 according to the following reaction scheme

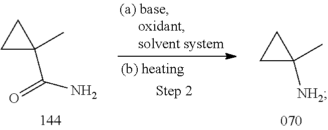

The second scheme comprising three steps:

Step 1 comprising forming a reaction mixture comprising compound 900, CHX₃, a base, a solvent system, and a phase transfer catalyst, and reacting the mixture to form a reaction product mixture comprising compound 905 according to the following reaction scheme

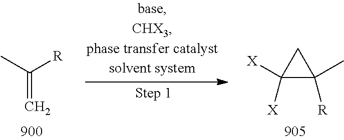

wherein R is CONH2; and
wherein each X is independently selected from Cl, Br, and I; and

Step 2 comprising forming a reaction mixture comprising compound 905, a reducing agent, and a solvent system, and reacting the reaction mixture to form a reaction product mixture comprising compound 144 according to the following reaction scheme

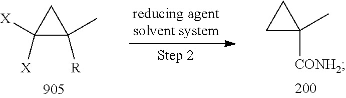

and

Step 3 comprising forming a reaction mixture comprising compound 144, a base, an oxidant, and a solvent system, and reacting the reaction mixture to form a reaction product mixture comprising a N-halocarboxamide intermediate, followed by heating the reaction product mixture comprising the N-halocarboxamide intermediate to form a reaction product mixture comprising compound 070 according to the following reaction scheme

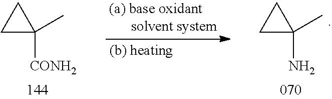

In one aspect, compound 905a, an intermediate compound useful for preparation of compound 070 and having the following structure:

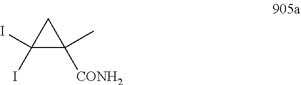

Another aspect of the disclosure is directed to a process for the preparation of compound 070, the process comprising a first, a second and a third scheme, the first scheme comprising:

Step 1 comprising forming a reaction mixture comprising compound 403, an acid, and a solvent system, and reacting the reaction mixture to form a reaction product mixture comprising compound 070 according to the following scheme

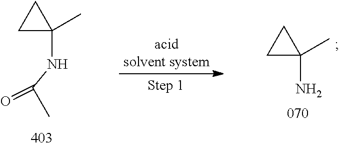

the second scheme comprising two steps, comprising:

Step 1 comprising forming a reaction mixture comprising compound 994, an activator, and a solvent system, reacting the reaction mixture to form a reaction product mixture comprising compound 403 according to the following scheme

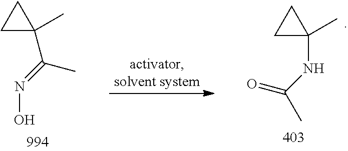

Step 2 comprising forming a reaction mixture comprising compound 403, and acid, and a solvent system, and reacting the reaction mixture to form a reaction product mixture comprising compound 070 according to the following scheme

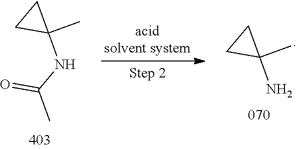

The third scheme comprising three steps, comprising:

Step 1 comprising forming a reaction mixture comprising compound 079, a source of hydroxylamine, a base, and a solvent system, and reacting the reaction mixture to form a reaction product mixture comprising compound 994 according to the following reaction scheme

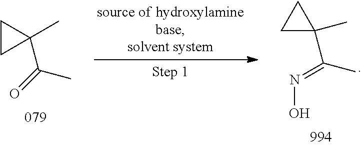

Step 2 comprising forming a reaction mixture comprising compound 994, an activator, and a solvent system, and reacting the reaction mixture to form a reaction product mixture comprising compound 403 according to the following

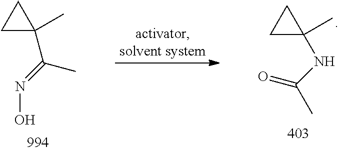

Step 3 comprising forming a reaction mixture comprising compound 403, an acid, and a solvent system, and reacting the reaction mixture to form a reaction product mixture comprising compound 070 according to the following reaction scheme

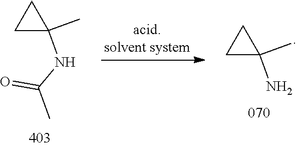

Another aspect of the disclosure is directed to a process for the preparation of compound 070. The process comprises forming a reaction mixture comprising acetonitrile, ethyl magnesium halide, a titanium reagent, and a solvent, reacting the reaction mixture, adding an acid to the reaction mixture, and further reacting the reaction mixture to form a reaction product mixture comprising compound 070 according to the following scheme

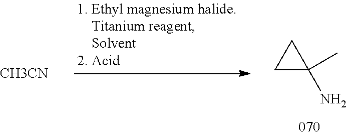

Another aspect of the disclosure is directed to a process for the preparation of compound 093a, the process comprising steps 1 to 4.

Step 1 comprises forming a reaction mixture comprising compound 339, a source of Br, optionally a photo—or thermally sensitive radical initiator, and a solvent system, and reacting the reaction mixture under photochemical conditions to form a reaction product mixture comprising compound 181a, according to the following reaction scheme

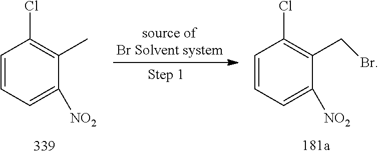

Step 2 comprises forming compound 378 by the (i) the combination of steps 2(a) and 2(b) or by (ii) step 2.

Step 2(a) comprises forming a reaction mixture comprising compound 181a, a base, and a solvent system, and reacting the reaction mixture to form a reaction product mixture comprising compound 050 according to the following reaction scheme

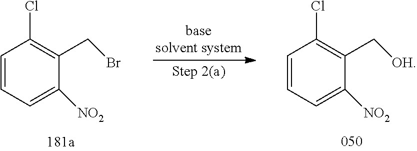 [SMILES: O=[N+]([O-])c1cccc(Cl)c1CBr ; O=[N+]([O-])c1cccc(Cl)c1CO]

Step 2(b) comprises forming a reaction mixture comprising compound 050, an oxidizing reagent, a phase transfer catalyst, and a solvent system, and reacting the reaction mixture to form a reaction product mixture comprising compound 378 according to the following reaction scheme

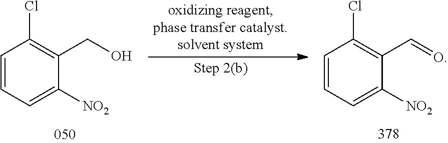 [SMILES: O=[N+]([O-])c1cccc(Cl)c1CO ; O=Cc1c(Cl)cccc1[N+](=O)[O-]]

Step 2 comprises forming a reaction mixture comprising compound 181a, an oxidizing reagent, and a solvent system, and reacting the reaction mixture to form a reaction product mixture comprising compound 378 according to the following reaction scheme

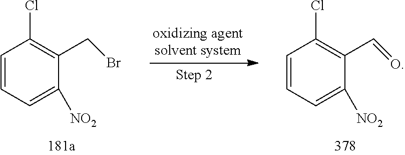 [SMILES: O=[N+]([O-])c1cccc(Cl)c1CBr ; O=Cc1c(Cl)cccc1[N+](=O)[O-]]

Step 3 comprises forming a reaction mixture comprising compound 378, 3-aminopyridine, an acid catalyst, and a solvent system, and reacting the reaction mixture to form a reaction product mixture comprising compound 003a according to the following reaction scheme

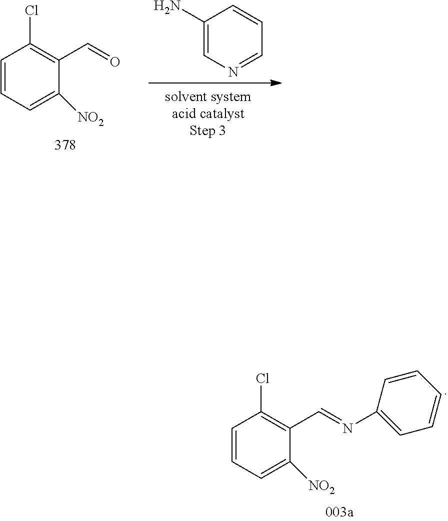 [SMILES: O=Cc1c(Cl)cccc1[N+](=O)[O-] ; Nc1cccnc1 ; O=[N+]([O-])c1cccc(Cl)c1/C=N/c1ccccc1]

Step 4 comprises forming a reaction mixture comprising compound 003a, a phosphine or a phosphite, and a solvent system, and reacting the reaction mixture to form a reaction product mixture comprising compound 093a according to the following reaction scheme

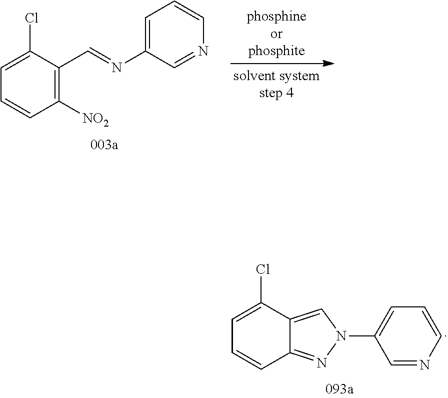 [SMILES: O=[N+]([O-])c1cccc(Cl)c1/C=N/c1cccnc1 ; Clc1cccc2nn(-c3cccnc3)cc12]

wherein the phosphine is selected from tri (C₁₋₄) alkyl phosphine and triaryl phosphine and the phosphite is selected from tri (C₁₋₄) alkyl phosphite and triaryl phosphite.

Another aspect of the disclosure is directed to a process for the preparation of compound 093a, the process comprising steps 1 to 7.

Step 1 comprises forming a reaction mixture comprising compound 150, an acid, 3-aminopyridine, and a solvent system, and reacting the reaction mixture to form a reaction product mixture comprising compound 086 according to the following reaction scheme

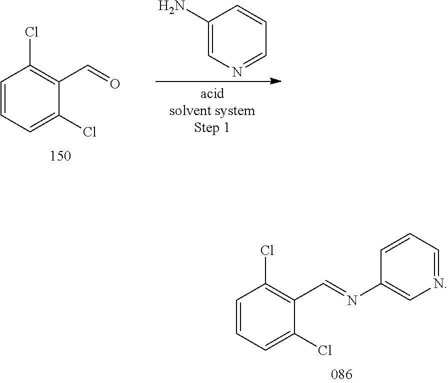 [SMILES: O=Cc1c(Cl)cccc1Cl ; Nc1cccnc1 ; Clc1cccc(Cl)c1/C=N/c1cccnc1]

Step 2 comprises forming a reaction mixture comprising compound 086, a reducing agent, and a solvent system, and reacting the reaction mixture to form a reaction product mixture comprising compound 084 according to the following reaction scheme

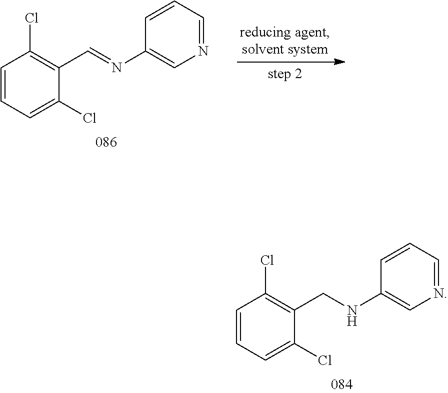 [SMILES: Clc1cccc(Cl)c1/C=N/c1cccnc1 ; Clc1cccc(Cl)c1CNc1cccnc1]

Step 3 comprises forming a reaction mixture comprising compound 084, a reagent for converting an amine moiety to a nitrosamine moiety, an acid, and a solvent system, and reacting the reaction mixture to form a reaction product mixture comprising compound 085 according to the following reaction scheme

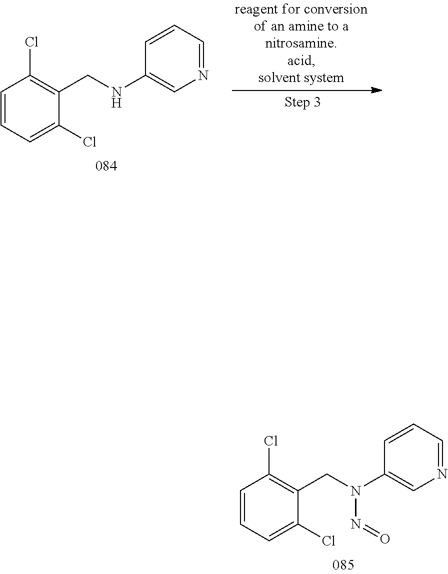 [SMILES: Clc1cccc(Cl)c1CNc1cccnc1 ; O=NN(Cc1c(Cl)cccc1Cl)c1cccnc1]

Step 4 comprises (a) forming a reaction mixture comprising compound 085, a reductant, a base, and a solvent system, and reacting the reaction mixture followed by (a) acidification to form a reaction product mixture comprising compound 048 according to the following reaction scheme

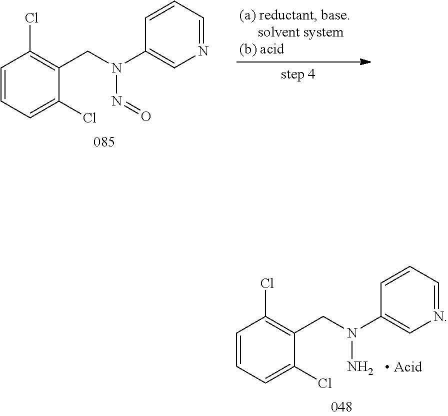 [SMILES: O=NN(Cc1c(Cl)cccc1Cl)c1cccnc1 ; NN(Cc1c(Cl)cccc1Cl)c1cccnc1]

Step 5 comprises forming a reaction mixture comprising compound 048, acetic anhydride, a base, and a solvent system, and reacting the reaction mixture to form a reaction product mixture comprising compound 083 according to the following reaction scheme

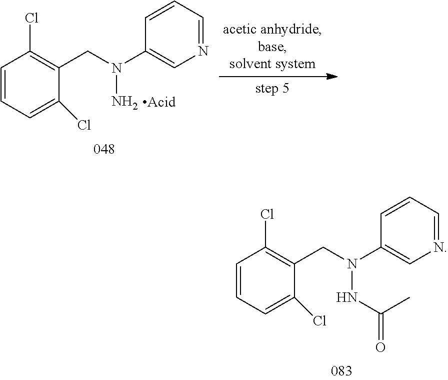 [SMILES: NN(Cc1c(Cl)cccc1Cl)c1cccnc1 ; CC(=O)NN(Cc1c(Cl)cccc1Cl)c1cccnc1]

Step 6 comprises forming a reaction mixture comprising compound 083, a ligand, a transition metal catalyst, a base, and a solvent system, and reacting the reaction mixture to form a reaction product mixture comprising compound 082 according to the following reaction scheme

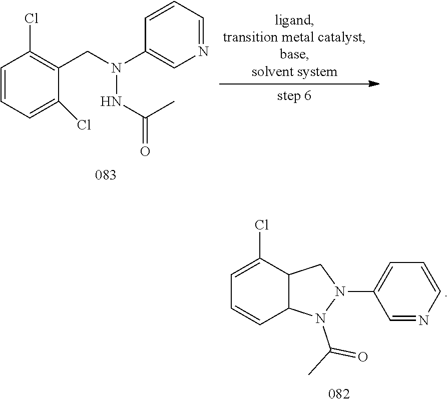 [SMILES: CC(=O)NN(Cc1c(Cl)cccc1Cl)c1cccnc1 ; CC(=O)N1C2C=CC=C(Cl)C2CN1c1cccnc1]

Step 7 comprises forming a reaction mixture comprising compound 082, a base, and a solvent system, and reacting the reaction mixture to form a reaction product mixture comprising compound 093a according to the following

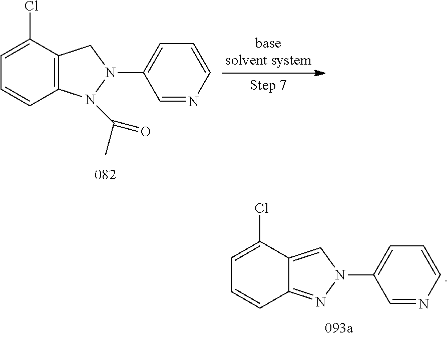 [SMILES: CC(=O)N1c2cccc(Cl)c2CN1c1cccnc1 ; Clc1cccc2nn(-c3cccnc3)cc12]

Another aspect of the disclosure is directed to a process for the preparation of compound 093a, the process comprising steps 1-3.

Step 1 comprises forming a reaction mixture solution comprising compound 150, acetohydrazine, a solvent system, and an organic acid, and reacting the reaction mixture to form a reaction product mixture comprising compound 197 according to the following reaction scheme

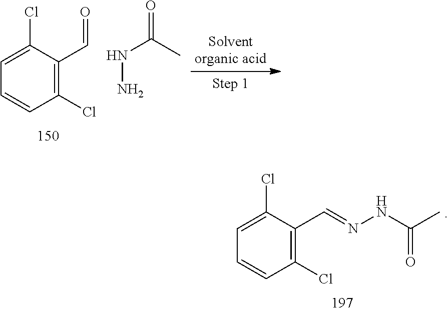

Step 2 comprises forming a reaction mixture comprising compound 197, a reducing agent, and a solvent system, and reacting the reaction mixture to form a reaction product mixture comprising compound 040 according to the following reaction scheme

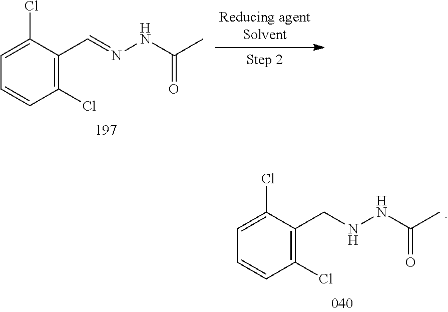

Step 3 comprises forming a reaction mixture comprising compound 040, 3-bromopyridine, a ligand, a transition metal catalyst, and a solvent system, and reacting the reaction mixture to form a reaction product mixture comprising compound 093a according to the following reaction scheme

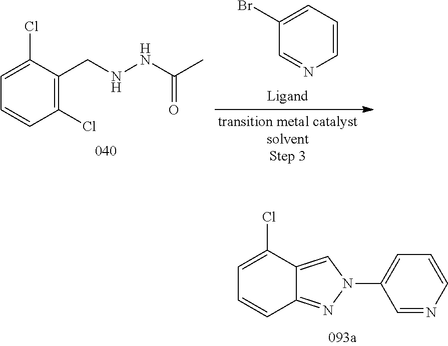 [SMILES: CC(=O)NNCc1c(Cl)cccc1Cl ; Brc1cccnc1 ; Clc1cccc2nn(-c3cccnc3)cc12]

Another aspect of the disclosure is directed to a process for preparing compounds 093a or 093b according to a first or second scheme.

Another aspect of the disclosure is directed to an alternative procedure to compound 093 according to a first or second scheme.

The first scheme for the preparation of compounds 093a or 093b comprises steps 1 to 2.

Step 1 comprises forming a reaction mixture comprising compounds 181a or 181b, compound 520, and a solvent system, and reacting the reaction mixture to form a reaction product mixture comprising compounds 182a or 182b, respectively, according to the following reaction scheme.

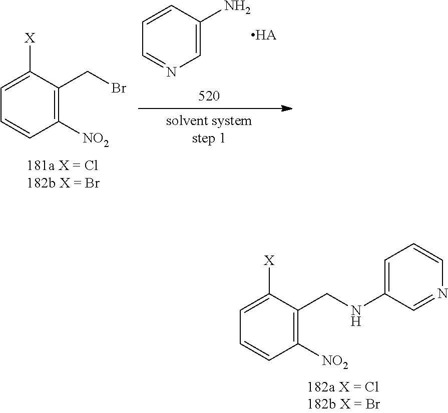

Step 2 comprises forming a reaction mixture comprising compounds 182a or 182b, a reductant, a base, and a solvent system, and reacting the reaction mixture to form a reaction product mixture comprising compounds 093a or 093b, respectively, according to the following reaction scheme

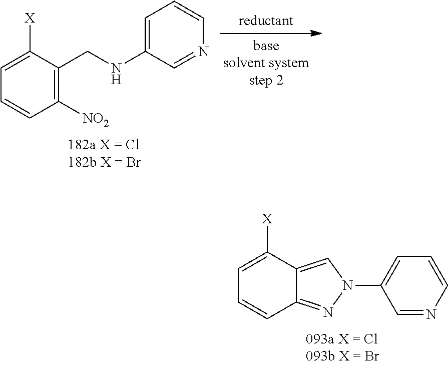

The second such scheme for preparing compounds 093a or 093b comprises steps 1 to 3.

Step 1 comprises forming a reaction mixture comprising compounds 114a or 114b, an oxidizing agent, and a solvent system, and reacting the reaction mixture to form a reaction product mixture comprising compounds 115a or 115b, respectively, according to the following reaction scheme.

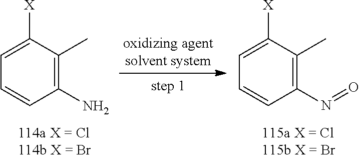

Step 2 comprises forming a reaction mixture comprising compounds 115a or 115b, a source of bromine or a source of chlorine, and a solvent system and reacting the reaction mixture under photochemical conditions to form a reaction product mixture comprising compound 116a, 116b, 116c, or 116d, according to the following reaction scheme.

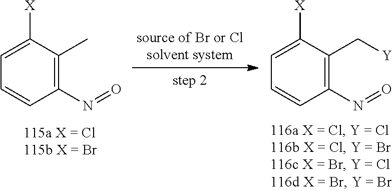

Step 3 comprises forming a reaction mixture comprising compound 116a, 116b, 116c, or 116d, compound 520, and a solvent system and reacting the reaction mixture to form a reaction product mixture comprising compound 093a, or 93b, respectively, according to the following reaction scheme.

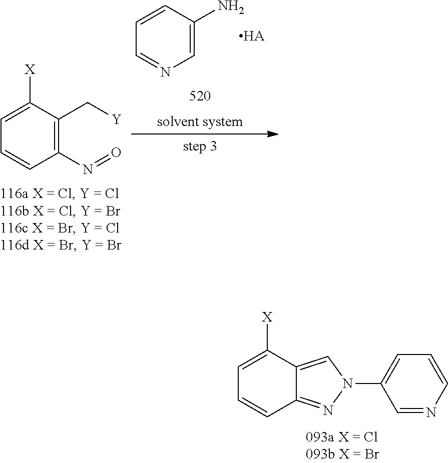

Another aspect of the disclosure is directed to an alternate process to prepare compound 182a or 182b. The process comprises forming a reaction mixture comprising compound 003a or 003b, a reducing agent, and a solvent system, and reacting the reaction mixture to form a reaction product mixture comprising compound 182a or 182b, respectively, according to the following reaction scheme.

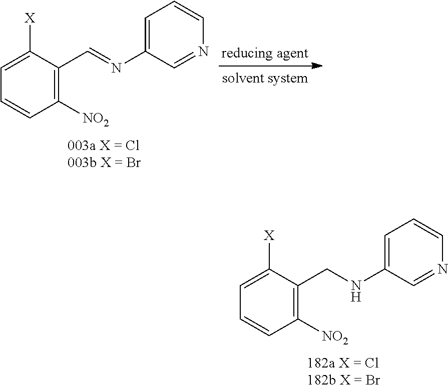

Another aspect of the disclosure is directed to a process for the preparation of compound 061. The process comprises forming a reaction mixture comprising compound 093a or 093b, CO, a catalyst, a ligand, a base, and a solvent system, and reacting the reaction mixture to form a reaction product mixture comprising compound 061 according to the following reaction scheme

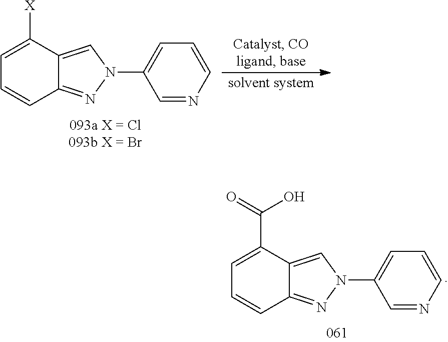

Another aspect of the disclosure is directed to a process for the preparation of compound 038 according to a first, second, or third scheme.

The first such scheme for preparing compound 038 comprises steps 1 to 3.

Step 1 comprises forming a reaction mixture comprising compound 400, an oxidizing agent, and a solvent system, and reacting the reaction mixture to form a reaction product mixture comprising compound 500 according to the following reaction scheme

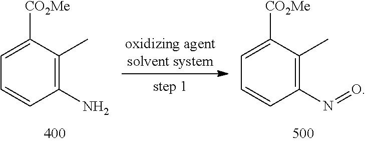

Step 2 comprises forming a reaction mixture comprising compound 500, a source of bromine or a source of chlorine, and a solvent system, and reacting the reaction mixture under photochemical conditions to form a reaction product mixture comprising compound 510a or 510b, according to the following reaction scheme

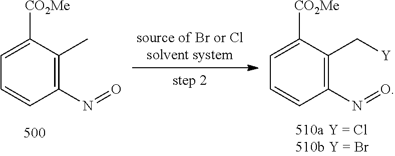

Step 3 comprises forming a reaction mixture comprising compound 510a or 510b, compound 520, and a solvent system, and reacting the reaction mixture to form a reaction product mixture comprising compound 038 according to the following reaction scheme

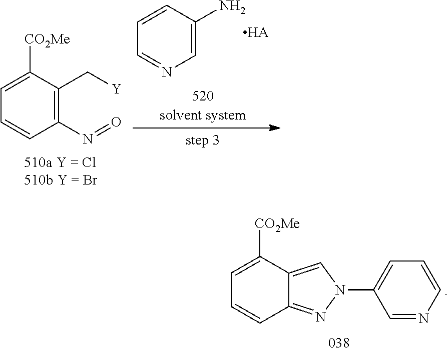

The second such scheme for preparing compound 038 comprises steps 1 to 3.

Step 1 comprises forming a reaction mixture comprising compound 400, a source of bromine or a source of chlorine, and a solvent system, and reacting the reaction mixture by exposure to a source of light to form a reaction product mixture comprising compound 410 according to the following reaction scheme

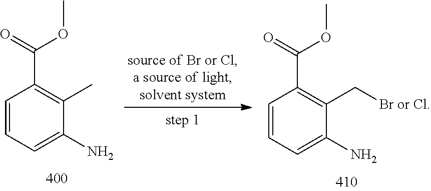

Step 2 comprising forming a reaction mixture comprising compound 410, compound 420, an acid, and a solvent system, and reacting the reaction mixture to form a reaction product mixture comprising compound 430 according to the following reaction scheme

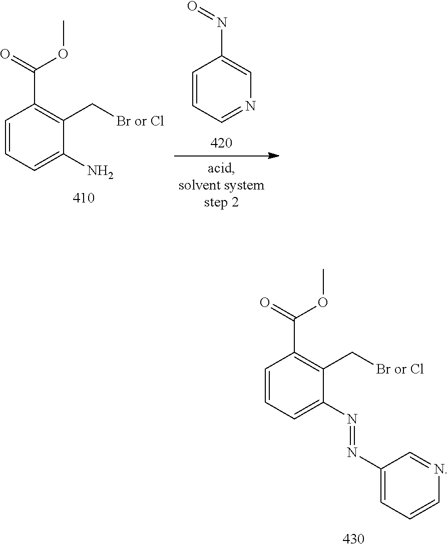

Step 3 comprises forming a reaction mixture comprising compound 430, a strong acid, and a solvent system, and reacting the reaction mixture to form a reaction product mixture comprising compound 038 according to the following

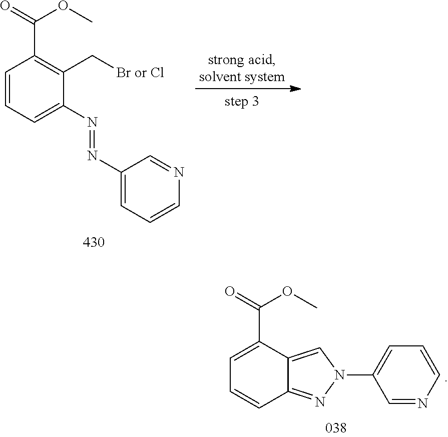

The third such scheme for preparing compound 038 comprises forming a reaction mixture comprising compound 093a or 093b, a catalyst, a ligand, CO, a base, and a solvent system, and reacting the reaction mixture to form a reaction product mixture comprising compound 038 according to the following reaction scheme

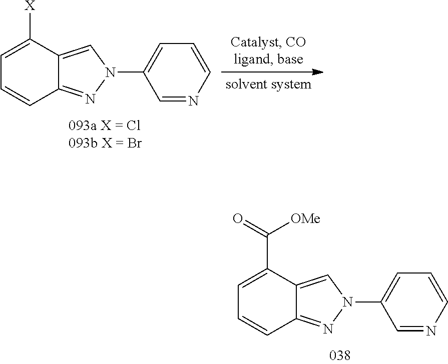

Another aspect of the disclosure is directed to a process for the preparation of compound 061, the process comprising forming a reaction mixture comprising compound 038, a base, and a solvent system, and reacting the reaction mixture to form a reaction product mixture comprising compound 061 according to the following reaction scheme

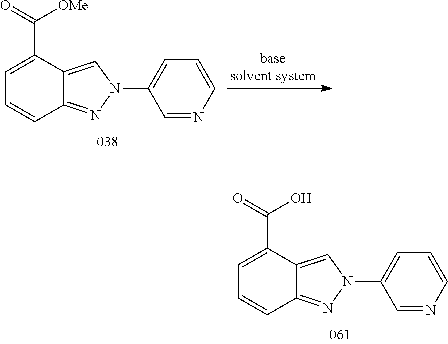 [SMILES: COC(=O)c1cccc2nn(-c3cccnc3)cc12 ; O=C(O)c1cccc2nn(-c3cccnc3)cc12]

Another aspect of the disclosure is directed to a process for preparing compound 038, the process comprising forming a reaction mixture comprising compound 061, an acid, optionally an additive, and a solvent system containing methanol, and reacting the reaction mixture to form a reaction product mixture comprising compound 038 according to the following reaction scheme

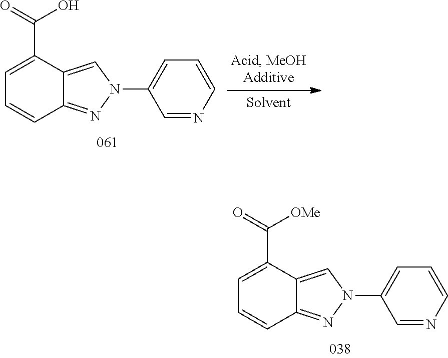 [SMILES: O=C(O)c1cccc2nn(-c3cccnc3)cc12 ; COC(=O)c1cccc2nn(-c3cccnc3)cc12]

In one aspect, an intermediate compound useful for preparation of compound 093a and having the following structure:

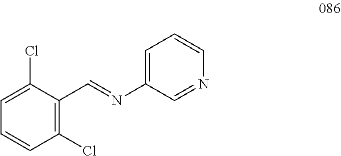 [SMILES: Clc1cccc(Cl)c1/C=N/c1cccnc1]

In one aspect, an intermediate compound useful for preparation of compound 093a and having the following structure:

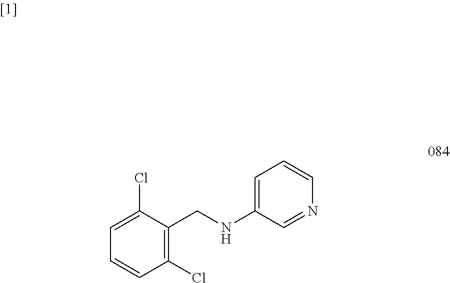 [SMILES: Clc1cccc(Cl)c1CNc1cccnc1]

In one aspect, an intermediate compound useful for preparation of compound 093a and having the following structure.

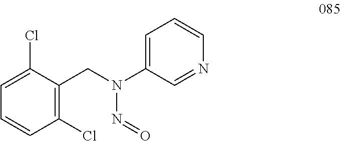 [SMILES: O=NN(Cc1c(Cl)cccc1Cl)c1cccnc1]

In one aspect, an intermediate compound useful for preparation of compound 093a and having the following structure and the hydrochloride salt:

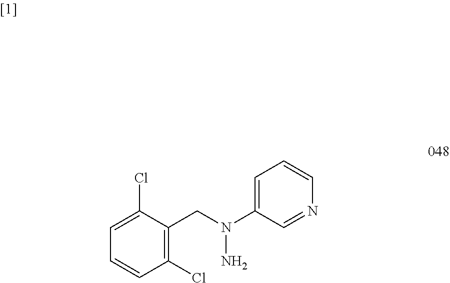 [SMILES: NN(Cc1c(Cl)cccc1Cl)c1cccnc1]

In one aspect, an intermediate compound useful for preparation of compound 093a and having the following structure:

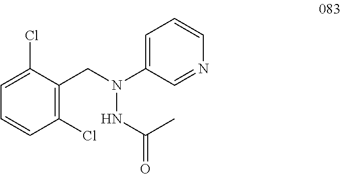 [SMILES: CC(=O)NN(Cc1c(Cl)cccc1Cl)c1cccnc1]

In one aspect, an intermediate compound useful for preparation of compound 093a and having the following structure:

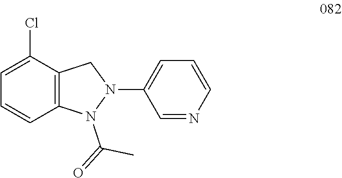 [SMILES: CC(=O)N1c2cccc(Cl)c2CN1c1cccnc1]

In one aspect, an intermediate compound useful for preparation of compound 093a and having the following structure

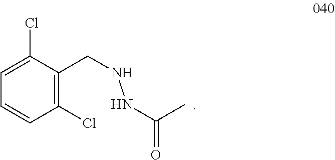

In one aspect, a compound having the structure of compound 520a:

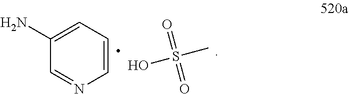

In one aspect, a compound having the structure of compounds 182a or 182b

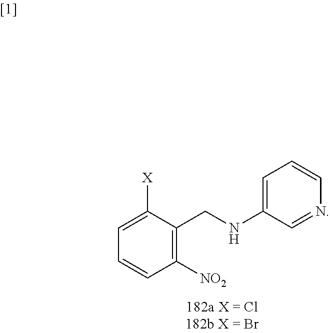

In one aspect, a compound having the structure of compound 115b:

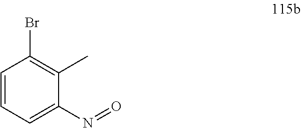

In one aspect, a compound having the structure of compound 116a-d:

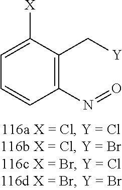

In one aspect, a compound having the structure of compound 500:

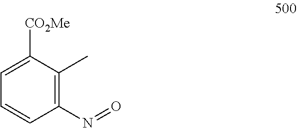

In one aspect, a compound having the structure of compound 510a or 510b:

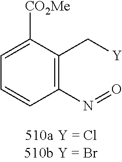

Another aspect of the disclosure is directed to a process for preparing compound 092, the process comprising steps 1 and 2.

Step 1 comprises forming a reaction mixture comprising compound 061, a chlorinating reagent, and a solvent system, and reacting the reaction mixture to form a reaction product mixture comprising compound 930 according to the following reaction scheme

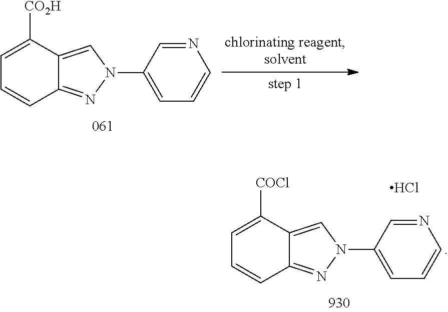 [SMILES: O=C(O)c1cccc2nn(-c3cccnc3)cc12 ; O=C(Cl)c1cccc2nn(-c3cccnc3)cc12 ; Cl]

Step 2 comprises forming a reaction mixture comprising compound 930, compound 070, an organic base, and a solvent system, and reacting the reaction mixture to form a reaction product mixture comprising compound 092 according to the following reaction scheme

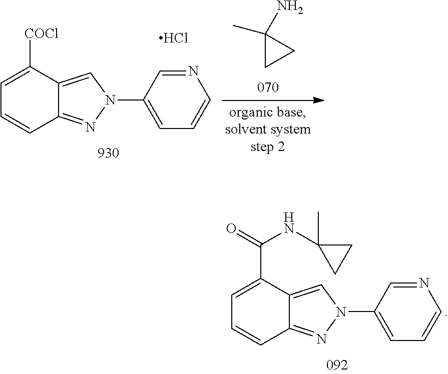

Another aspect of the disclosure is directed to a process for preparing compound 092, the process comprising forming a reaction mixture comprising compound 093a or 93b, compound 070, a catalyst, a ligand, CO, a base, and a solvent system, and reacting the reaction mixture to form a reaction product mixture comprising compound 092 according to the following reaction scheme.

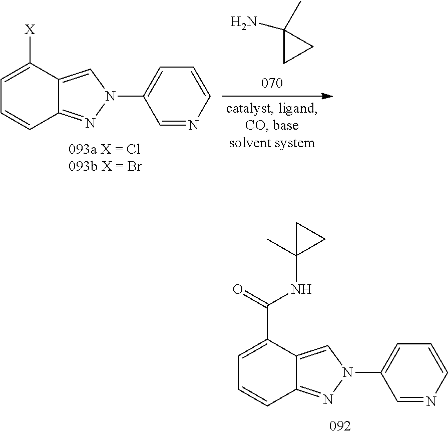

Another aspect of the disclosure is directed to a process for preparing compound 092, the process comprising forming a reaction mixture comprising compound 061, compound 070, an activator, a base, and a solvent system, and reacting the reaction mixture to form a reaction product mixture comprising compound 092 according to the following reaction scheme

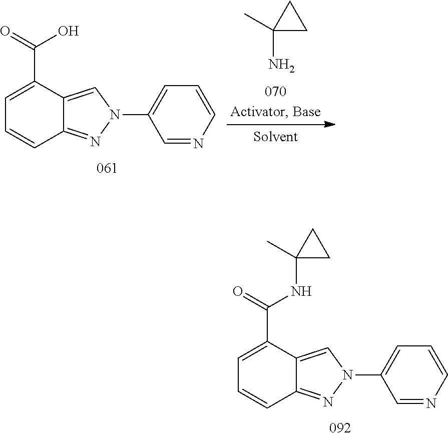

Another aspect of the disclosure is directed to a process for preparing compound 092, the process comprising forming a reaction mixture comprising compound 038, compound 070, a base, and a solvent system, and reacting the reaction mixture to form a reaction product mixture comprising compound 092 according to the following reaction scheme

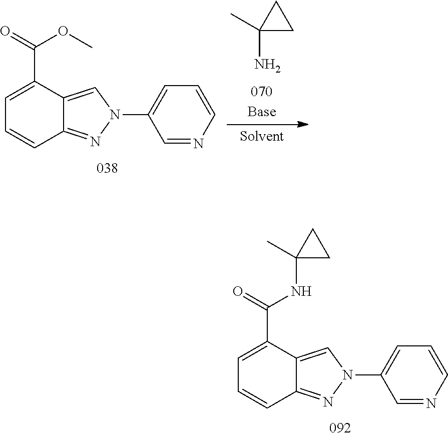

Another aspect of the disclosure is directed to a process for preparing a compound 092 salt, the process comprising forming a reaction mixture comprising compound 092, a solvent system, an acid, and reacting the reaction mixture to form a reaction product mixture comprising compound 092 salt according to the following

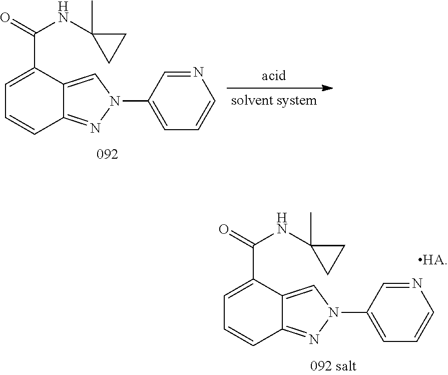

Another aspect of the disclosure is directed to a polymorph of N-(1-methylcyclopropyl)-2-(3-pyridinyl)-2H-indazole 4-carboxamide designated Form A characterized by a X-ray powder diffraction pattern in accordance with FIG. 1.

Another aspect of the disclosure is directed to a polymorph of N-(1-methylcyclopropyl)-2-(3-pyridinyl)-2H-indazole 4-carboxamide designated Form B characterized by a X-ray powder diffraction pattern in accordance with FIG. 2.

### BRIEF DESCRIPTION OF THE DRAWINGS

FIG. 1 shows an XRPD pattern for N-(1-methylcyclopropyl)-2-(3-pyridinyl)-2H-indazole 4-carboxamide Form A prepared according to the examples of the present disclosure.

FIG. 2 shows an XRPD pattern for N-(1-methylcyclopropyl)-2-(3-pyridinyl)-2H-indazole 4-carboxamide Form B prepared according to the examples of the present disclosure.

### DETAILED DESCRIPTION OF THE DISCLOSURE

The disclose is generally directed to improved processes for the preparation of N-(1-methylcyclopropyl)-2-(3-pyridinyl)-2H-indazole 4-carboxamide, designated as compound 092 herein and of the structure

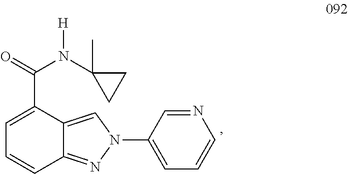 [SMILES: CC1(NC(=O)c2cccc3nn(-c4cccnc4)cc23)CC1]

and intermediates thereof.

As used herein the term “inorganic base” generally includes sodium, potassium, ammonium, calcium, magnesium, iron, zinc, copper, manganese, and aluminum salts. Non-limiting examples include phosphates such as dipotassium monohydrogen phosphate, potassium dihydrogen phosphate, tripotassium phosphate, disodium monohydrogen phosphate, sodium dihydrogen phosphate, trisodium phosphate, diammonium monohydrogen phosphate, ammonium dihydrogen phosphate and triammonium phosphate; acetates such as potassium acetate, sodium acetate and ammonium acetate; formates such as potassium formate and sodium formate; carbonates such as cesium carbonate, potassium carbonate, sodium carbonate, potassium hydrogen carbonate, and sodium hydrogen carbonate; ammonium hydroxide; and alkali metal hydroxides such as lithium hydroxide, sodium hydroxide and potassium hydroxide. The inorganic bases may be used singly, or in combination of two or more kinds thereof.

As used herein, the term “organic base” generally includes primary, secondary, and tertiary amines, substituted amines including naturally occurring substituted amines, cyclic amines and basic ion exchange resins, such as pyridine, isopropylamine, trimethylamine, diethylamine, triethylamine, triethanolamine, diisopropylamine, ethanolamine, 2-diethylaminoethanol, trimethylamine, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, and polyamine resins. The organic bases may be used alone, or in combination with one or more kinds thereof.

As used herein the term “organometallic base” generally includes organolithium, organomagnesium, organoaluminum, or organozinc compounds. Non-limiting examples include organolithiums such as methyl lithium, n-butyl lithium, sec-butyl lithium, tert-butyl lithium; organomagnesiums such as methyl magnesium chloride, methyl magnesium bromide, methyl magnesium iodide, ethyl magnesium chloride, ethyl magnesium bromide iso-propyl magnesium chloride, iso-propyl magnesium bromide; organoaluminums such as trimethyl aluminum, triethylaluminum, triisobutylaluminum. Diisobutylaluminum hydride; organozincs such as dimethyl zinc or diethyl zinc. The organometallic bases may be used alone or in combination with any of the preceding bases.

As used herein, the term “inorganic acid” refers to an acid comprising an inorganic component. Examples of inorganic acids include mineral acids including, but not limited to hydrochloric acid, hydrobromic acid, hydroiodic acid, sulfuric acid, nitric acid, phosphoric acid, and boric acid. The inorganic acid may be used alone, or in combination of two or more kinds thereof.

As used herein, the term “organic acid” refers to an organic compound that acts an acid. Examples of organic acids include but are not limited carboxylic acids. Examples of organic acids include, but are not limited to, formic acid, oxalic acid, malonic acid, succinic acid, glutaric acid, butanedioic acid, adipic acid, tartaric acid and citric acid. The organic acid may be used alone, or in combination of two or more kinds thereof.

As used herein, the term “non-polar solvent” refers to a solvent without significant partial charges on any atoms or a solvent where polar bonds are arranged in such a way that the effect of their partial charges cancel out. Non-limiting examples of non-polar solvents include pentane, hexane, heptane, cyclopentane, cyclohexane, benzene, toluene, xylenes, 1,4-dioxane, dichloromethane (“DCM”), methyl tert-butyl ether (“MTBE”), chloroform, carbon tetrachloride, diethyl ether, and combinations thereof.

As used herein, the term “aprotic solvent” refers to a solvent that does not donate hydrogen. As used herein, “polar aprotic solvent” refers to a solvent having high dielectric constants and high dipole movements and that lack an acidic hydrogen. Non-limiting examples of polar aprotic solvents include tetrahydrofuran (“THF”), methyl tetrahydrofuran (“Me-THF”), ethyl acetate (“EA”), acetone, dimethylformamide (“DMF”), dimethylacetamide (“DMAc”), acetonitrile (“ACN”), cyclopentylmethyl ether (“CPME”), petroleum ether, N-methyl-2-pyrrolidone (“NMP”), trifluorotoluene, chlorobenzene, anisole, and dimethyl sulfoxide (“DMSO”). In some aspects, the aprotic solvent is a low molecular weight ester. Non-limiting examples of aprotic low molecular weight ester solvents include methyl acetate, ethyl acetate, n-propyl acetate, i-propyl acetate, i-butyl acetate, propylene glycol methyl ether acetate, monoethyl ether acetate, and combinations thereof.

As used herein, the term “polar protic solvent” refers to a solvent having a labile hydrogen bound to an oxygen atom or a nitrogen atom. Non-limiting examples of polar protic solvents include formic acid, n-butanol, i-propanol, n-propanol, ethanol, methanol, acetic acid, water, and combinations thereof.

As used herein, the term “solvent” refers to a non-polar solvent, an aprotic solvent, a polar protic solvent, and combinations thereof.

As used herein, the term “solvent system” refers to a solvent or mixture of solvents. Solvent systems may comprise, or predominantly comprise, the indicated solvent or combination of solvents. Solvent systems may further comprise residual solvent from one or more preceding process steps.

As used herein the term “reducing agent” refers to a compound that donates electrons, either directly or through a hydride (“H—)”. Non-limiting examples of reducing agents include sodium, potassium, zinc, iron, magnesium, sodium borohydride, potassium borohydride, p-toluenesulfonic acid, sodium bis(2-methoxyethoxy)aluminum hydride, sodium bisulfite, sodium hydrogensulfite, sodium hydrosulfite, sodium tetrahydroborate, potassium tetrahydroborate, sodium triacetoxyborohydride, trichlorosilane, triphenylphosphite, triethylsilane, trimethylphosphine, triphenylphosphine, diborane, diethoxymethylsilane, diisobutylaluminum hydride, diisopropylaminoborane, lithium aluminum hydride, and lithium triethylborohydride.

As used herein, the term “oxidizing agent” refers to a compound that receives an electron. Non-limiting examples of oxidizing agents include: hypochlorite, chlorate, and perchlorate; peroxides such as H₂O₂; O₂; O₃; N₂O; halogens such as F₂, Cl₂, Br₂, and I₂; HNO₃; KNO₃; H₂SO₄; H₂S₂O₈; and H₂SO₅.

As used herein, the terms “halogen”, “halo” and “halide” are used interchangeably and refer to any of F, Cl, Br, and I.

As used herein, the term “alkyl” refers to a saturated linear or branched chain monovalent hydrocarbon group. The alkyl group is suitably one to six carbon atoms (C₁₋₆), one to four carbon atoms (C₁₋₄), or one to three carbon atoms (C₁₋₃). Non-limiting examples of alkyl groups include methyl (Me, —CH₃), ethyl (Et, —CH₂CH₃), 1-propyl (n-Pr, —CH₂CH₂CH₃), 2-propyl (i-Pr, —CH(CH₃)₂), 1-butyl (n-Bu, —(CH₂)₃CH₃), and 1,1-dimethylethyl (t-buyl, (CH₃)₃C—).

As used herein, the term “source of light” refers to visible light such as supplied by sunlight or a light source such as a xenon lamp, a halogen lamp, a fluorescent lamp, a diode or a mercury lamp. A filter that cuts wavelengths other than a necessary wavelength is within the scope of the disclosure.

As used herein, the term “photocatalyst” refers to substances exhibiting a photocatalytic activity by exposure of light having an energy higher than a predetermined band gap. In some aspects, the photocatalyst may be a visible light photocatalyst, non-limiting examples of which include 4CzIP, CZS1, CzS2, 2Cz-DPS, 2TCz-DPSN, fac-Ir(ppy)₃, [Ir(ppy)2(dtbbpy)]PF₆, [Ir(dF(CF₃)ppy)₂(bpy)]PF₆, [Ir(dF(CF₃)ppy)₂(dtbbpy)]PF₆, [Ir(dF(Me)ppy)₂(bpy)]PF₆, [Ir(F(Me)ppy)₂(bpy)]PF₆, [Ru(bpy)₃](PF₆)₂, [Acr-Mes]ClO₄, Eosin Y, and Rose Bengal. In some aspects, the photocatalyst may be one or a combination of metal oxide semiconductors, such as—for instance and without limitation-titanium oxide, tungsten oxide, zinc oxide, tin oxide, iron oxide, bismuth oxide, bismuth vanadate and strontium titanate.

As used herein, the term “photochemical conditions” refers to using a source of light or near-visible electromagnetic radiation to promote a reaction.

Salts of the compounds disclosed herein are within the scope of the present disclosure. Salts include both acid and base addition salts. “Acid addition salt” refers to salts formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, malonic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, and salicyclic acid. “Base addition salt” refers to salts formed with an organic or inorganic base.

As used herein, the term “predominantly” means greater than 50%, at least 75%, at least 90% or at least 95% on a population %, w/w %, w/v % or v/v % basis.

As used herein, the term “chemical processing aid” means a chemical added directly to the reaction mixture or is present in a mixture and is used to aid in processing and its function is such that it does not remain in the product.

As used herein, the term “transition metal catalyst” refers to a substance containing at least one element from groups III through XI of the periodic table which exhibits catalytic activity. Non limiting examples include iron catalysts, nickel catalysts, palladium catalysts, platinum catalysts, or copper catalysts. Non limiting examples of iron catalysts where X stands for halogen include Fe, FeX₂, FeX₃, Fe(acac)₃, Fe(NO₃)₃, or Fe(OTf)₃. Non limiting examples of nickel catalysts include Raney Ni, Ni/C, Ni/Si Ni, NiX₂, NiX₂·nH₂O, NiX₂(DME), NiX₂(diglyme), (bpy)NiX₂, Ni(OTf)₃, Ni(acac)₂, Ni(COD)₂, Ni(CO)₄, (dppe)NiX₂, (dppp)NiX₂, (dppb)NiX₂, (dppf)NiX₂, (dcype)NiX₂, (dcypp)NiX₂, (dcypb)NiX₂, (binap)NiX₂, (bpy)NiX₂ or solvates thereof. Non limiting examples of palladium catalysts where X stands for halogen include Pd, Pd/C, Pd/Si, Pd/BaSO₄, Pd(dba)₂, Pd₂(dba)₃, Pd(PPh₃)₄ PdX₂, Pd(OAc)₂, Pd(OBz)₂, [Pd(allyl)X]₂, Pd(MeCN)₂X₂, (COD)PdX₂, (2-methylallyl) palladium chloride dimer, Pd(OTf)₂, (PPh₃)₂PdX₂, (PCy₃)PdX₂, (PfBu₃)₂Pd, Pd[(o-tol)₃P]₂, trans-Di(μ-acetato)bis[o-(di-o-tolyl-phosphino)benzyl]dipalladium(II), Pd(amphos)X₂, (dppe)PdX₂, (dppp)PdX₂, (dppb)PdX₂, (dppf)PdX₂, (dppf)PdX₂·DCM, (dcype)PdX₂, (dcypp)PdX₂, (dcypb)PdX₂, (binap)PdX₂, (xantphos)PdX₂, (dpephos)PdX₂ or solvates thereof. Non limiting examples of copper catalysts where X stands for halogen include CuX, CuX₂, CuCN, Cu(OTf)₂, CuO, Cu₂O, CuBr·DMS or solvates thereof. In some aspects, the transition metal catalyst may act as a precursor to an active catalyst species which optionally may be preformed and charged into the reaction or generated in situ. In some aspects, the transition metal catalyst is optionally used with a ligand. In some such aspects, the ligand may optionally be precomplexed with the transition metal before addition into the reaction or the ligand and transition metal catalyst complexed in situ. In some aspects, the transition metal catalyst may be used alone or in combination with any of the preceding transition metal catalysts.

As used herein, the term “diphosphine ligand” refers to a substance containing two phosphino groups connected by a backbone. The diphosphine ligand is able to chelate to a transition metal catalyst in a bidentate fashion. Non limiting examples include 1,1-bis(diphenylphosphino)methane, 1,2-bis(diphenylphosphino)ethane, 1,3-bis(diphenylphosphino)propane, 1,4-bis(diphenylphosphino)butane, O-isopropylidene-2,3-dihydroxy-1,4-bis(diphenylphosphino)butane, 2,3-bis(diphenylphosphino)butane, 2,2′-bis(diphenylphosphino)-1,1′-binaphthyl, 1,2-Bis(diphenylphosphino)benzene, 4,5-Bis(diphenylphosphino)-9,9-dimethylxanthene, bis[(2-diphenylphosphino)phenyl]ether, 4,4′-bi-1,3-benzodioxole-5,5′-diylbis(diphenylphosphane), 5,5′-Bis[di(3,5-di-tert-butyl-4-methoxyphenyl)phosphino]-4,4′-bi-1,3-benzodioxole, (R,R)-(−)-2,3-Bis(tert-butylmethylphosphino) quinoxaline, (S,S)-(−)-2,3-Bis(tert-butylmethylphosphino) quinoxaline (R)-1-[(SP)-2-(dicyclohexylphosphino) ferrocenyl]ethyldi-tbutylphosphine, (+)-1,2-Bis[(2R,5R)-2,5-diethylphospholano]ethane 1,1′-bis(diphenylphosphino) ferrocene, 1,2-bis(2,5-dimethylphospholano)benzene, 1,2-bis(dicyclohexylphosphino) ethane, 1,3-bis(dicyclohexylphosphino) propane, 1,4-bis(dicyclohexylphosphino) butane, 1,2-bis(dicyclopentylphosphino) ethane, 1,3-bis(dicyclopentylphosphino) propane, or 1,4-bis(dicyclopentylphosphino) butane, 1,3-bis(di-tert-butylphosphino) propane, 1,1-bis(dimethylphosphino) methane 1,2-bis(dimethylphosphino) ethane, 1,3-bis(dimethylphosphino) propane, 1,4-bis(dimethylphosphino) butane, 1,2-bis(diethylphosphino) ethane, 1,3-bis(diethylphosphino) propane, 1,4-bis(diethylphosphino) butane, 1,2-bis(diamylphosphino) ethane, 1,3-bis(diamylphosphino) propane, 1,4-bis(diamylphosphino) butane In some aspects, the diphosphine ligands are used as bisphosphonium salts that are then converted to the free base diphosphine in situ. In some such aspects, the diphosphonium salts are hydrochloride, hydrobromide, hydroiodide, tetrafluoroborate salts or some combination thereof. In some aspects, the diphosphine ligand may optionally be added separately from the transition metal catalyst or added precomplexed to the transition metal catalyst. In some aspects, the diphosphine may optionally in situ be oxidized to the monophosphine oxide, which acts as the ligand for an active catalyst species. In some aspects, the diphosphine ligand may be used alone or in combination with any of the preceding diphosphine ligands.

Some aspects of the disclosure are directed to a process for the preparation of compound 775, the process comprising the following scheme:

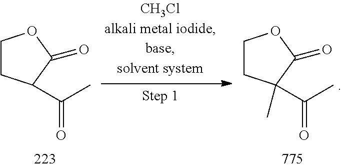

The process comprises forming a reaction mixture comprising compound 223 (3-acetyldihydrofuran-2 (3H)-one), CH₃Cl, an alkali metal iodide, a base, and a solvent system, and reacting the reaction mixture to form a reaction product mixture comprising compound 775 (3-acetyl-3-methyldihydrofuran-2 (3H)-one).

In some aspects, CH₃Cl is present in a stoichiometric excess with respect to compound 223.

In some aspects, the alkali metal iodide is selected from NaI, KI, and LiI. In some such aspects, the alkali metal iodide is KI. The alkali metal is present in a catalytic amount.

In some aspects, the base is an inorganic base. In some such aspects, the base is a weak inorganic base. In some such aspects, the base is a carbonate. In some such aspects, the base is selected from sodium carbonate, sodium bicarbonate, potassium carbonate, and potassium bicarbonate. In some aspects, the base may be present in a stoichiometric excess with respect to compound 223.

In some aspects, the solvent system comprises or predominantly comprises an aprotic solvent. In some such aspects, a suitable solvent can be selected from among acetone, methyl t-butyl ether, acetonitrile, 1,4-dioxane, tetrahydrofuran, and isopropyl acetate. In some such aspects, the solvent system comprises acetonitrile, or the solvent system comprises acetone. In some such aspects, the solvent system comprises or predominantly comprises acetone.

In some aspects, the reaction is run at reflux. The reaction may be monitored for completion by methods known in the art such as ¹H NMR (CDCl₃), high performance liquid chromatography (“HPLC”), or ultraperformance liquid chromatography (“UPLC”).

The process for preparing compound 775 provides for good selectivity and yield to compound 775. The process for preparing compound 775 provides for elimination of certain expensive and hazardous reagents known in the art, such as Mel, organic bases such as sodium amylate or sodium methoxide, and sodium metal.

Some aspects of the disclosure are directed to a process for the preparation of compound 200 (1-methylcyclopropane-1-carboxylic acid) according to a first scheme, a second scheme, or a third scheme.

Various methods for cyclopropanation are known in the prior art. See Ebner, et al., “Cyclopropanation Strategies in Recent Total Synthesis”, Chem. Rev. 2017, 117, 18, 11651-11679; de Meijere, et al., “Small Ring Compounds in Organic Synthesis VI”, Topics in Current Chemistry, January 2000, DOI: 10.1007/3-540-48255-5; and Rappaport, Ed., “The Chemistry of the Cyclopropyl Group”, Vol 1, Patai's Chemistry of Functional Groups, 1987. Each of those references is incorporated by reference herein.

The first scheme for preparing compound 200 comprises three steps.

Step 1 of the first scheme for preparing compound 200 comprises forming a reaction mixture comprising compound 775 (3-acetyl-3-methyldihydrofuran-2 (3H)-one), HCl, and a solvent system, and reacting the reaction mixture to form a reaction product mixture comprising compound 069 (5-chloro-3-methylpentan-2-one) according to the following reaction scheme:

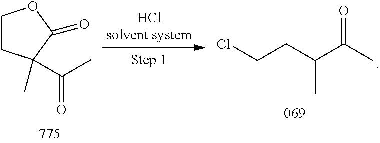

In some aspects, HCl is concentrated HCl. In some aspects, the HCl is hydrogen chloride gas. In some aspects, the solvent system comprises or predominantly comprises an aprotic solvent, water, or a combination thereof. In some aspects, the aprotic solvent is DCM. In some aspects, the solvent system comprises less than 70 w/w % water. In some aspects, concentrated HCl suitably has an HCl concentration of from about 30 w/w % to about 38 w/w %.

Step 2 of the first scheme for preparing compound 200 comprises forming a reaction mixture comprising compound 069, a base, and a solvent system, and reacting the reaction mixture to form a reaction product mixture comprising compound 079 (methyl-(1-methylcyclopropyl)-ketone) according to the following reaction scheme

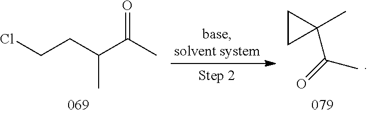

In some aspects, the base is a strong inorganic base. In some such aspects, the base is an alkali metal hydroxide. In some such aspects, the base is selected from sodium hydroxide and potassium hydroxide.

In some aspects, the solvent system comprises or predominantly comprises a polar protic solvent, water, or a combination thereof.

Step 3 of the first scheme for preparing compound 200 comprises forming a reaction mixture comprising compound 079, an oxidizing agent, optionally a base, and a solvent system, and reacting the reaction mixture to form a reaction product mixture comprising compound 200 according to the following reaction scheme

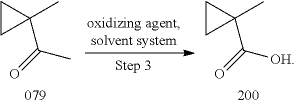

In some aspects, the oxidizing agent is selected from sodium hypochlorite, sodium hypobromite, bromine, or chlorine. In some such aspects, the oxidizing agent is sodium hypochlorite. In some aspects, the concentration of sodium hypochlorite is as obtained in a commercially available solution.

In some aspects, optionally a base is present. In some aspects, the base is selected from sodium hydroxide or potassium hydroxide.

In some aspects, the solvent system comprises or predominantly comprises water.

In some aspects, any of compounds 775, 069, 079, and 200 are optionally isolated from the reaction product mixture. In some aspects any two, or all, of the sequential reactions from compound 223 to compound 775, from compound 775 to compound 069, from compound 069 to compound 079, and from compound 079 to compound 200, are carried forward to the next step without isolation or purification.

The second scheme for preparing compound 200 comprises two steps.

Step 1 of the second scheme for preparing compound 200 comprises forming a reaction mixture comprising compound 900, CHX₃, a base, a solvent system, and a phase transfer catalyst (PTC), and reacting the reaction mixture to form a reaction product mixture comprising compound 905 according to the following reaction scheme

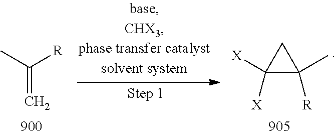

R is selected from CO₂CH₃, CO₂CH₂CH₃, COOH, and CN. In some such aspects, R may be CO₂CH₃ or CO₂CH₂CH₃. Each X is independently selected from Cl, Br, and I. In some such aspects, each X is Cl;

when R is CO₂CH₃, CO₂CH₂CH₃, or CN, the process further comprises Step 1b forming a reaction mixture comprising compound 905, a base, and a solvent system, and reacting the reaction mixture to form a reaction product mixture comprising compound 906 according to the following reaction scheme

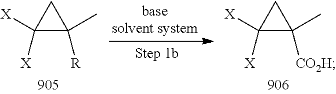

Step 2 comprises forming a reaction mixture comprising compound 906, a reducing agent, and a solvent system, and reacting the reaction mixture to form a reaction product mixture comprising compound 200 according to the following reaction scheme

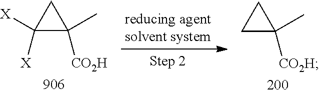

When R is COOH, the process comprises Step 2′, Step 2′ comprising forming a reaction mixture comprising compound 905, a reducing agent, and a solvent system, and reacting the reaction mixture to form a reaction product mixture comprising compound 200 according to the following scheme

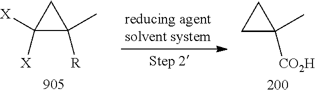

In some aspects of Step 1, the base is a strong inorganic base. In some such aspects, the base is an alkali metal hydroxide. In some such aspects, the base is selected from sodium hydroxide and potassium hydroxide. In some aspects, the base is present in molar excess over compound 900.

In some aspects of Step 1, the CHX₃ reagent is chloroform, bromoform, or iodoform. In one aspect, the CHX₃ reagent is chloroform. In some aspects, chloroform is present in molar excess over compound 900.

Phase transfer catalysts (“PTC”) are known in the art. In some aspects of Step 1, the PTC is selected from ammonium halide salts, crown ethers, and PEGs. Non-limiting examples of suitable PTCs include triethylbenzylammonium chloride, triethylbenzylammonium bromide, tetraethylammonium bromide, tetrabutylammonium bromide, and ethyltrimethylammonium iodide. In one aspect, the PTC is triethylbenzylammonium chloride. The PTC is generally present in a catalytic amount.

In some aspects of Step 1, suitable solvents include protic solvents, aprotic solvents, and combinations thereof. Non-limiting examples of solvents include water, hexane, pentane, heptane, benzene, toluene, chlorobenzene, dichloromethane, and combinations thereof.

The reaction temperature may be suitably selected to achieve desired purity and yield in a commercially acceptable time. Reaction completion may suitably be measured by in-process testing as described elsewhere herein.

When R is COOCH₃, COOCH₂CH₃, or nitrile, the process further comprises Step 1b forming a reaction mixture comprising compound 905, a base, and a solvent system, and reacting the reaction mixture to form a reaction product mixture comprising compound 906 according to the following reaction scheme.

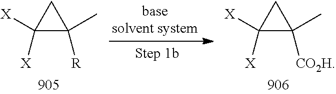

In some aspects of Step 1b, the base is a strong inorganic base. In some such aspects, the base is an alkali metal hydroxide. In some such aspects, the base is selected from sodium hydroxide and potassium hydroxide.

In some aspects of Step 1b, the solvent system suitably comprises or predominantly comprises a polar protic solvent. In some such aspects, the solvent system comprises or predominantly comprises a C₁₋₄ alcohol, such as methanol or ethanol. In some aspects, the solvent system comprises methanol. In some such aspects, the solvent system comprises water. In some such aspects, the solvent system comprises water and a C₁₋₄ alcohol mixture.

In some aspects, the reaction product mixture may be optionally worked up. For instance, water may be added and the pH adjusted to less than 3, such as about 1-2, with a strong acid such as HCl. The resultant mixture may be extracted with a solvent, such as a non-polar solvent (e.g., toluene) to extract compound 906. The extraction mixture may then be optionally evaporated (e.g., under vacuum) to isolate compound 906.

Step 2 of the second scheme for preparing compound 200 comprises forming a reaction mixture comprising compound 906, a reducing agent, and a solvent system, and reacting the reaction mixture to form a reaction product mixture comprising compound 200 according to the following reaction scheme

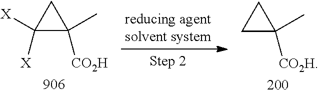

In some step 2 aspects, the transformation of compound 906 to compound 200 is carried out under hydrodehalogenation conditions, comprising forming a reaction mixture comprising compound 906, H₂, a metal catalyst, a base, and a solvent system, and reacting the reaction mixture to form a reaction product mixture comprising compound 200. Reaction temperature and pressure may be suitably selected to achieve commercially acceptable yield, purity, and throughput.

In some aspects, a suitable solvent is selected from among methanol, ethanol, 1-propanol, isopropanol, t-butanol, isobutanol, sec-butanol, 1-hexanol, 2-ethyl-1-hexanol, 2-octanol, benzyl alcohol, n-octane, cyclohexane, xylene, tetrahydrofuran, dioxane, water, monoglyme, diglyme, ethyleneglycol, N,N-dimethylformamide, N,N-dimethylsulfoxide, triethylamine, pyridine, and combinations thereof. In some aspects, the solvent system comprises a polar protic solvent. In some aspects, the polar protic solvent comprises a C₁₋₄ alcohol. In one aspect, the solvent is t-butanol.

In some aspects, the reaction mixture comprises a base. Suitable bases include sodium hydroxide, potassium hydroxide, lithium hydroxide, calcium hydroxide, sodium carbonate, potassium carbonate, sodium bicarbonate, potassium bicarbonate, sodium ethoxide, potassium ethoxide, sodium tert-butoxide, potassium tert-butoxide, triethylamine, pyridine, ethylenediamine, and combinations thereof. In some such aspects, the base is an alkali metal hydroxide. In some such aspects, the base is selected from sodium hydroxide and potassium hydroxide. In some aspects, the base is an alkali metal alkoxide. In some aspects, the base is selected from sodium tert-butoxide and potassium tert-butoxide. In some aspects, the base is potassium tert-butoxide. In some aspects, the base is in molar excess over compound 906.

In some aspects, the metal catalyst is a Pt, Pd, Rh, or Ru catalyst. In some aspects, the metal catalyst is Pd/C, Rh/Al₂CO₃, Pd/CaCO₃, Pd/Pb/CaCO₃, or Pt/Al₂O₃. In one aspect, the metal catalyst is Pd/C.

The reaction product mixture may be optionally worked up. For instance, and without limitation, the reaction product mixture may be filtered and the filtrate may be evaporated to remove solvent. The resulting mixture may be diluted with water and acidified with strong acid (e.g., HCl) to a pH of less than 3, such as from about 1 to about 2. The resultant mixture may be extracted with a solvent (e.g., DCM) to extract compound 200. In some aspects, compound 200 may be isolated by evaporation of the solvent to yield compound 200.

In some step 2′ aspects, the transformation of compound 905 to compound 200 is carried out under reducing metal conditions, comprising forming a reaction mixture comprising compound 905, a reducing metal, and a solvent system, and reacting the reaction mixture to form a reaction product mixture comprising compound 200.

In some aspects, the solvent system comprises a polar aprotic solvent, a polar protic solvent, or a combination thereof. In some such aspects, a suitable solvent is selected from among tert-butanol, acetic acid, water, isopropanol, THF, ethylene glycol, tetramethylethylenediamine, N,N-dimethylaniline, DMF, diethanolamine, ethylenediamine, triethylamine, ammonium hydroxide, and combinations thereof. In some aspects, the solvent system may suitably comprise or predominantly comprise a polar aprotic solvent. In some aspects, the solvent is tetrahydrofuran (THF). In some aspects, the solvent system comprises or predominantly comprises a polar aprotic solvent (e.g., THF) and a polar protic solvent (such as a C₁₋₄ alcohol (e.g., methanol or ethanol)), water, and combinations thereof.

In some aspects, the reducing metal is Na, K, Ca, Mg, or zinc. In some such aspects the reducing agent is selected form Na and Zn. In some such aspects, the reducing agent is sodium metal. In some aspects, the reducing agent is in molar excess over compound 905.

In some aspects, when the reducing metal is zinc, the solvent is acetic acid. In some aspects, when the reducing metal is zinc, a base is optionally present. In some aspects, the base is an inorganic base. In some such aspects, the base is an alkali metal hydroxide. In some such aspects, the base is selected from sodium hydroxide and potassium hydroxide. In some aspects, the base can also be used as a solvent, for example, triethylamine. In some aspects, the base is in molar excess over compound 905 from step 1.

In some aspects, the reducing agent may be added in portions during the course of the reaction. In some aspects, the reducing agent may be added continuously or semi-continuously during the course of the reaction.

Compound 200 may be isolated by methods known in the art such as solvent removal. In some aspects, the reaction product mixture may be optionally worked up. For instance, a solution containing compound 200 may be acidified, extracted with a solvent and isolated by methods known in the art.

In some aspects, any of compounds 905, compound 906, and 200 are optionally isolated from the reaction product mixture. In some aspects any two, or all, of the sequential reactions from compound 900 to compound 905, from compound 905 to compound 906, and from compound 906 to compound 200, are carried forward to the next step without isolation or purification.

The third scheme for preparing compound 200 comprises two steps.

Step 1 of the third scheme for preparing compound 200 comprises forming a reaction mixture comprising acetic acid, compound 100 (methyl 4-chloro-2-methylenebutanoate), a base, a photocatalyst, and a solvent system, and reacting the reaction mixture by exposure to light emitted from at least one light emitting source to form a reaction product mixture comprising compound 110 (methyl 1-methylcyclopropane-1-carboxylate) according to the following reaction scheme

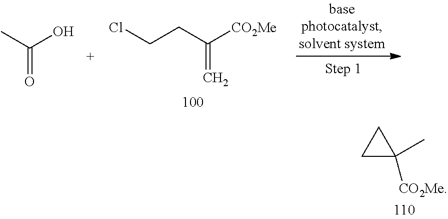

The solvent system may suitably comprise or predominantly comprises a polar or nonpolar solvent. In some such aspects, the solvent system comprises a polar aprotic solvent or the solvent system comprises dimethyl formamide (DMF). In some aspects, the polar solvent comprises or predominantly comprises DMF.

In some aspects, the base is an inorganic base. In some such aspects, the base is a carbonate. In some such aspects, the base is Cs₂CO₃. In some aspects, the base is an organic base.

In some aspects, the photocatalyst is a visible light photocatalyst. Non-limiting examples of photocatalysts within the scope of the disclosure include 4CzIP, CZS1, CzS2, 2Cz-DPS, 2TCz-DPSN, fac-Ir(ppy)₃, [Ir(ppy)2(dtbbpy)]PF₆, [Ir(dF(CF₃)ppy)₂^{(bpy)}]PF₆, [Ir(dF(CF₃)ppy)₂(dtbbpy)]PF₆, [Ir(dF(Me)ppy)₂(bpy)]PF₆, [Ir(F(Me)ppy)₂(bpy)]PF₆, [Ru(bpy)₃](PF₆)₂, [Acr-Mes]ClO₄, Eosin Y, and Rose Bengal. In some aspects, the photocatalyst is 4CzIP or Ir(ppy)₂(dtbbpy)PF₆.

In some aspects, the light source is a blue light emitting diode.

Step 2 of the third scheme for preparing compound 200 comprises forming a reaction mixture comprising compound 110, a solvent system, and a base, and reacting the reaction mixture to form a reaction product mixture comprising compound 200 according to the following reaction scheme

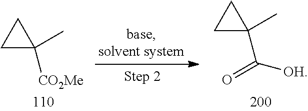

In some aspects, the base is an inorganic base. In some such aspects, the base is an alkali metal hydroxide. In some such aspects, the base is selected from sodium hydroxide and potassium hydroxide.

In some aspects, the solvent system suitably comprises or predominantly comprises a polar solvent. In some such aspects, the solvent system comprises or predominantly comprises a C₁₋₄ alcohol, such as methanol or ethanol. In some such aspects, the solvent system comprises water. In some such aspects, the solvent system comprises water and C₁₋₄ alcohol mixture.

The reaction product mixture may be optionally worked up. For instance, water may be added and the pH adjusted to less than 3, such as about 1-2, with a strong acid such as HCl. The resultant mixture may be extracted with a solvent, such as a non-polar solvent (e.g., toluene) to extract compound 200. The extraction mixture may then be optionally evaporated (e.g., under vacuum) to yield compound 200.

In some aspects, compounds 110 and 200 are optionally isolated from the reaction product mixture. In some aspects, the sequential reactions from compound 100 to compound 110 and from compound 110 to compound 200, are carried forward to the next step without isolation.

Some aspects of the disclosure are directed to a process for the preparation of compound 070, the process comprising two steps.

Step 1 comprises substep (a) comprising forming a reaction mixture comprising compound 200 (1-methylcyclopropanecarboxylic acid), a chlorinating reagent, and a solvent system, and reacting the reaction mixture to form a reaction product mixture comprising an acid chloride intermediate, followed by substep (b) comprising forming a reaction mixture by combining the reaction product mixture from substep (a) with an ammonia source and reacting the reaction mixture to form a reaction product mixture comprising compound 144 (1-methylcyclopropane carboxamide) according to the following reaction scheme

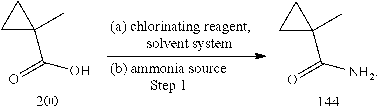

In some substep (a) aspects, the solvent system comprises or predominantly comprises an aprotic solvent. In some aspects, the solvent system comprises or predominantly comprises dichloromethane (DCM), toluene, acetonitrile or a combination thereof.

In some aspects, the substep (a) reaction mixture may further comprise a catalyst selected from DMF, 4-Dimethylaminopyridine (DMAP), triethylamine, N-Methyl-2-pyrrolidone (NMP), N-methylformanilide, N-formylpyridine, or pyridine.

In some aspects, the chlorinating reagent may be selected from thionyl chloride, triphosgene, or phosgene. In some such aspects, the chlorinating agent is thionyl chloride. The chlorination reagent is preferably in stoichiometric excess compared to compound 200.

In some aspects, the substep 1b solvent system predominantly comprises the solvent system from step 1a. In some aspects, the substep 1b solvent system predominantly comprises the combination of the solvent system from step 1a and a polar protic solvent. In some aspects, the substep 1b solvent system predominantly comprises a polar protic solvent. In either such aspect, the polar protic solvent may suitably be water, C₁₋₄ alcohol or a combination thereof, such as water, methanol, ethanol, or a combination thereof.

In some substep 1 (b) aspects, the ammonia source is selected from ammonia, ammonium hydroxide, and ammonia dissolved in a suitable organic solvent known in the art, such as methanol, ethanol, DCM, or toluene. In some such aspects, the ammonia source is selected form ammonia and ammonium hydroxide. In some aspects, ammonia is in molar excess to compound 200.

In some substep (b) aspects, the acid solution of compound 144 is added to the source of ammonia. In some aspects, the source of ammonia may be added to the reaction mixture.

Compound 144 may be optionally isolated from the reaction mixture by methods known in the art, such as solvent removal or filtration.

Step 2 comprises substep (a) comprising forming a reaction mixture comprising compound 144, a base, an oxidant, and a solvent system, and reacting the reaction mixture to form a reaction product mixture comprising a N-halocarboxamide intermediate, followed by substep (b) comprising heating the reaction product mixture comprising the N-halocarboxamide intermediate to form a reaction product mixture comprising compound 070 (1-methylcyclopropylamine) according to the following reaction scheme

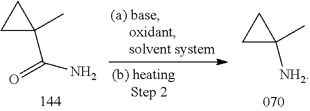

In some aspects, the oxidant is selected from Cl₂, NaOCl, Br₂, and NaOBr. In some particular aspects, the oxidant is NaOCl. In some other particular aspects, the oxidant is Br₂.

In some aspects, the base is an inorganic base. In some such aspects, the base is an alkali metal hydroxide. In some such aspects, the base is selected from sodium hydroxide and potassium hydroxide. In some aspects, the base is present in molar excess over compound 144.

In some aspects, the solvent system comprises or predominantly comprises a polar protic solvent. In some such aspects, the solvent system comprises or predominantly comprises water. In some such aspects, compound 144 is slurried in the solvent system.

In some aspects, a quenching reagent may be added to the reaction product mixture to quench the excess oxidant. In some aspects, the quenching agent is Na₂S₂O₃.

In some aspects of the disclosure, a representative example of the order of some of the reaction steps for certain acrylate-derived cyclopropyl compounds may be ordered as depicted below

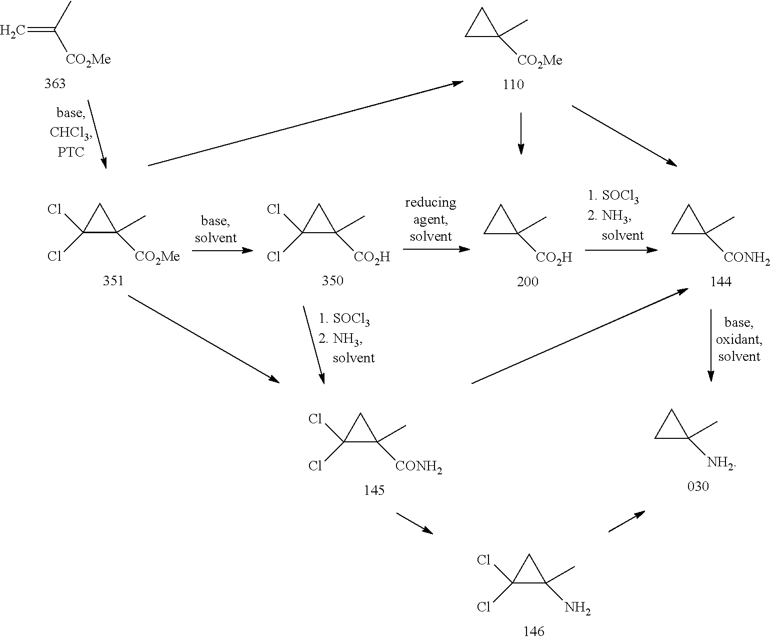

In such aspects, general conditions for the reactions in the above scheme have been previously described, except for those described below.

The reaction of compound 351 to form compound 110 may proceed according to the following reaction scheme

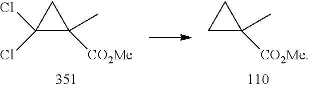

The reaction of compound 351 to form compound 110 may proceed according to conditions known in literature for dehalogenation of alkanes.

The reaction of compound 110 to form compound 200 may proceed according to the following reaction scheme

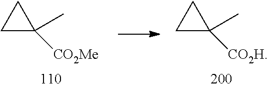

The reaction of compound 110 to form compound 144 may proceed according to the following reaction scheme

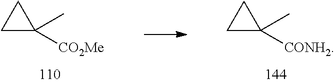

The reaction of compound 110 to form compound 144 may proceed according to conditions known in literature for ammonolysis of organic esters to amides.

The reaction of compound 351 to form compound 145 may proceed according to the following reaction scheme

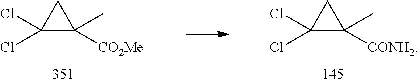

The reaction of compound 351 to form compound 145 may proceed according to conditions known in literature for ammonolysis of esters to amides.

The reaction of compound 350 to form compound 145 may proceed according to the following reaction scheme

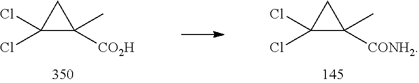

The reaction of compound 350 to form compound 145 may proceed according to conditions known in literature for ammonolysis of carboxyl groups to amides.

The reaction of compound 145 to form compound 146 may proceed according to the following reaction scheme

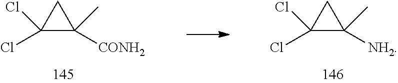

The reaction of compound 145 to form compound 144 may proceed according to the following reaction scheme

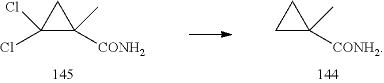

The reaction of compound 146 to form compound 070 may proceed according to the following reaction scheme

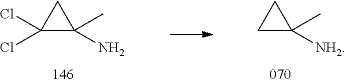

Some aspects of the disclosure are directed to a process for the preparation of compound 070, the process comprising three steps.

Step 1 comprises forming a reaction mixture comprising compound 079, a source of hydroxylamine, a base, and a solvent system, and reacting the reaction mixture to form a reaction product mixture comprising compound 994 (1-(1-methylcyclopropyl) ethan-1-one oxime) according to the following reaction scheme

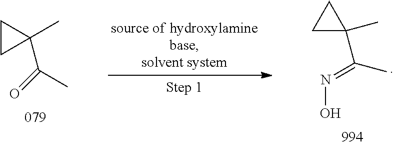

In some aspects, the source of amine is selected from hydroxylamine and hydroxylamine salts. In one aspect, the hydroxylamine salt is the HCl salt.

In some aspects, the base is selected from an inorganic base or an organic base. In some such aspects, the base is sodium acetate or potassium acetate.

In some aspects, the solvent system comprises or predominantly comprises a polar protic solvent. In some such aspects, the solvent system comprises or predominantly comprises a C₁₋₄ alcohol. In one aspect, the solvent system comprises or predominantly comprises methanol or ethanol.

In some aspects, the reaction product mixture comprises (E,Z)-1-(1-methylcyclopropyl) ethan-1-one oxime. In some aspects, the reaction product mixture comprises (E)-1-(1-methylcyclopropyl) ethan-1-one oxime. In some aspects, the reaction product mixture comprises (Z)-1-(1-methylcyclopropyl) ethan-1-one oxime.

Step 2 comprises forming a reaction mixture comprising compound 994, an activator, and a solvent system, and reacting the reaction mixture to form a reaction product mixture comprising compound 403 (N-(1-methylcyclopropyl) acetamide) according to the following reaction scheme

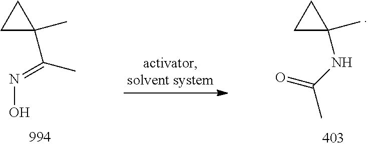

In some aspects, the activator is selected from tosyl chloride, cyanuric hydrochloride, thionyl chloride, sulfamic acid, phosphorus pentachloride, phosphorus pentoxide, triethylamine, inorganic bases, inorganic acids, organic acids, trimethylsilyl iodide, transition metal catalysts (such as zinc chloride), thiamine hydrochloride, alkylpyridinium salts, chloral, and combinations thereof. In some aspects, the activator is selected from tosyl chloride, cyanuric hydrochloride, thionyl chloride, and sulfamic acid. In some such aspects, the activator is tosyl chloride

In some aspects, the solvent system comprises or predominantly comprises a polar or nonpolar solvent. In some such aspects, the solvent system comprises or predominantly comprises acetonitrile.

Step 3 comprises forming a reaction mixture comprising compound 403, an acid, and a solvent system, and reacting the reaction mixture to form a reaction product mixture comprising compound 070 according to the following reaction scheme

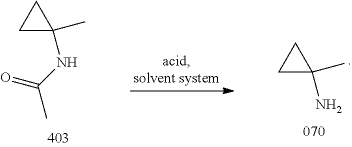

In some aspects, the acid is selected from an inorganic acid or an organic acid. In some such aspects, the acid is selected from among hydrochloric acid, sulfuric acid, phosphoric acid, acetic acid, and TsOH. In some such aspects, the acid is a mineral acid. In one aspect, the acid is H₂SO₄.

In some aspects, the solvent system comprises or predominantly comprises a polar protic solvent. In some such aspects, the solvent system comprises or predominantly comprises water.

In some aspects, any of compounds 994, 403, and 070 are optionally isolated from the reaction product mixture. In some aspects, two, or all, of the sequential reactions from compound 079 to compound 070 are carried forward onto the next step without isolation or purification.

In some aspects of the present disclosure, compound 070 may be prepared from acetonitrile according to the following reaction scheme

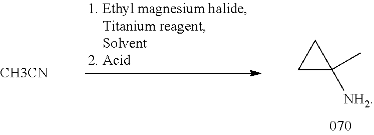

In some aspects, the ethyl magnesium halide reagent may be suitably selected from ethyl magnesium bromide and ethyl magnesium chloride.

In some aspects, the solvent may be suitably selected from diethyl ether, tetrahydrofuran, 2-methyltetrahydrofuran, methyl tert-butyl ether, tert-amyl methyl ether, and cyclopentyl methyl ether.

In some aspects, the titanium reagent is selected from titanium(IV) methoxide, titanium(IV) ethoxide, titanium(IV) propoxide, titanium(IV) isopropoxide, titanium(IV) butoxide, titanium(IV) tert-butoxide, titanium(IV) 2-ethylhexyloxide, and methyltitanium(IV) triisopropoxide.

In some aspects, the acid is a Lewis acid or a Bronsted acid. In some aspects, the Lewis acid is selected from boron trifluoride, boron trifluoride diethyl etherate, boron trifluroride tetrahydrofuran complex, boron trifluoride dibutyl etherate, boron trifluoride tert-butyl methyl etherate, boron trichloride, titanium (IV) chloride, aluminum trichloride, cerium (III) trichloride heptahydrate, zinc chloride, nickel (II) bromide trihydrate. In some aspects, the Bronsted acid is selected from sulfuric acid, phosphoric acid, and acetic acid.

A challenge in using this chemistry to form cyclopropanamines is the large amount of metal salts present in the reaction mass, which causes significant difficulty in post-reaction workup and product isolation steps. For example, polar cyclopropanamine products are water soluble and a typical extractive workup used to remove inorganic salts will result in significant product loss to the aqueous layer. Another challenge is the insoluble titanium dioxide that forms during post-reaction workup which coats reactor vessel walls and requires aggressive reactor cleanout protocols to remove. (Org. Process Res. Dev. 2021, 25, 2351; Org. Process Res. Dev. 2020, 24, 1735-1742; Org. Process Res. Dev. 2012, 16, 836).

In some aspects, a chemical processing aid is added after the reaction to facilitate product isolation. In some aspects, the chemical processing aid is selected from tartrate salts, such as potassium sodium tartrate, lactate salts, glycolate salts, ethylenediaminetetraacetate salts, triethanolamine, and citrate salts. In some aspects, the salts are generated from the corresponding acid upon treatment with a base. In some aspects, the processing aid is a flocculent. In some aspects the flocculent is selected from aluminum sulfate, ferric chloride, ferrous sulfate, ferric sulfate, sodium silicate, silicate salts, and silicate/kaolin clays or hydrates thereof.

Some aspects of the disclosure are directed to a process for the preparation of compound 093a (4-chloro-2-(pyridin-3-yl)-2H-indazole), the process comprising four steps.

Step 1 comprises forming compound 181a (2-(bromomethyl)-1-chloro-3-nitrobenzene) by forming a reaction mixture comprising compound 339 (1-chloro-2-methyl-3-nitrobenzene), a source of Br, optionally a photosensitive radical initiator, and a solvent system, and reacting the reaction mixture under photochemical conditions to form a reaction product mixture comprising compound 181a according to the following reaction scheme

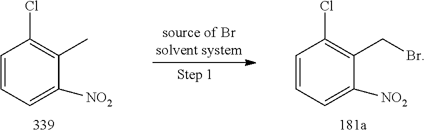

In some aspects, the source of Br is selected from N-bromosuccinimide, Br₂, or the combination of HBr and H₂O₂. In some aspects, the source of Br is N-bromosuccinimide.

In some aspects, the solvent system comprises or predominantly comprises at least one polar aprotic solvent. In some such aspects, the solvent system comprises or predominantly comprises dichloromethane (DCM) or a combination of DCM and water.

In some aspects, the reaction is promoted by a light source. In some aspects, the photochemical conditions relate to promoting the reaction using a visible light source. In some such aspects, the visible light is provided by about 400W of a light source.

In some aspects, a photosensitive or thermally activated radical initiator is used. In some such aspects the radical initiator is azobisisoutyronitrile.

Step 2 comprises forming compound 378 by the (i) the combination of steps 2(a) and 2(b) or by (ii) step 2.

(i). Step 2(a) comprises forming a reaction mixture comprising compound 181a, a base, and a solvent system, and reacting the reaction mixture to form a reaction product mixture comprising compound 050 ((2-chloro-6-nitrophenyl) methanol) according to the following reaction scheme

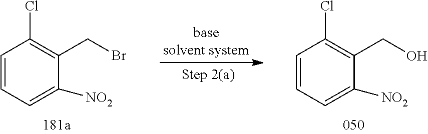 [SMILES: O=[N+]([O-])c1cccc(Cl)c1CBr ; O=[N+]([O-])c1cccc(Cl)c1CO]

In some aspects, the base is a weak base. In some aspects, the base is an inorganic base. In some aspects the base is a carbonate or a bicarbonate. In some such aspects, the base is selected from sodium carbonate, sodium bicarbonate, potassium carbonate, potassium bicarbonate, and magnesium carbonate.

In some aspects, the solvent system comprises or predominantly comprises at least one polar solvent system. In some such aspects, the solvent system comprises or predominantly comprises water. In some such aspects, the solvent system comprises or predominantly comprises acetonitrile. In some such aspects the solvent system comprises acetonitrile and water.

Step 2(b) comprises forming a reaction mixture comprising compound 050, an oxidizing reagent, a PTC, and a solvent system, and reacting the reaction mixture to form a reaction product mixture comprising compound 378 (2-chloro-6-nitro benzaldehyde) according to the following reaction scheme

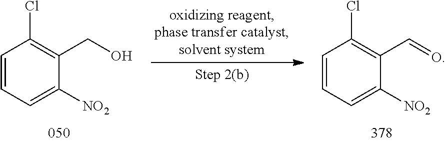 [SMILES: O=[N+]([O-])c1cccc(Cl)c1CO ; O=Cc1c(Cl)cccc1[N+](=O)[O-]]

In some aspects, the oxidizing reagent is NaOCl.

In some aspects, the phase transfer catalyst (PTC) is a quaternary ammonium salt. In some aspects the PTC is tetrabutylammonium bromide.

In some aspects, the solvent system comprises or predominantly comprises a non-polar solvent. In some such aspects, the solvent system comprises or predominantly comprises toluene.

(ii) Step 2 comprises forming a reaction mixture comprising compound 181a ((2-(bromomethyl)-1-chloro-3-nitrobenzene), an oxidizing reagent, and a solvent system, and reacting the reaction mixture to form a reaction product mixture comprising compound 378 according to the following reaction scheme

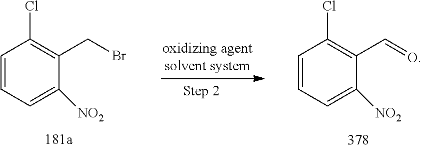 [SMILES: O=[N+]([O-])c1cccc(Cl)c1CBr ; O=Cc1c(Cl)cccc1[N+](=O)[O-]]

In some aspects, the oxidizing reagent is a N-oxide reagent. In some such aspects the reagent is trimethylamine-N-oxide or N-methylmorpholine N-oxide.

In some aspects, the solvent system comprises or predominantly comprises at least one polar solvent. In some such aspects, the solvent system comprises or predominantly comprises DMSO or DMF.

Step 3 comprises forming a reaction mixture comprising compound 378, 3-aminopyridine, an acid catalyst, and a solvent system, and reacting the reaction mixture to form a reaction product mixture comprising compound 003a (1-(2-chloro-6-nitrophenyl)-N-(pyridine-3-yl) methanimine) according to the following reaction scheme

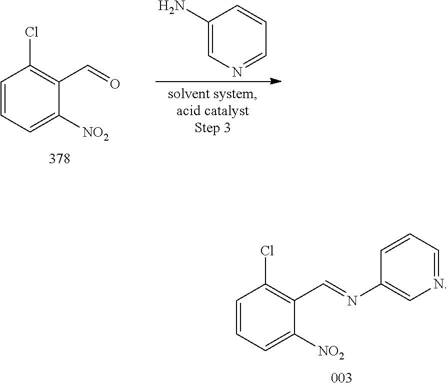 [SMILES: O=Cc1c(Cl)cccc1[N+](=O)[O-] ; Nc1cccnc1 ; O=[N+]([O-])c1cccc(Cl)c1/C=N/c1cccnc1]

In some aspects, the solvent system comprises or predominantly comprises a non-polar solvent. In some such aspects, the solvent system comprises or predominantly comprises toluene.

In some aspects, the acid is an organic acid. In some such aspects the acid is p-toluenesulfonic acid.

In some aspects, the reaction is run at reflux with concomitant azeotropic distillation of water.

Step 4 comprises forming a reaction mixture comprising compound 003a, a phosphine or a phosphite, and a solvent system, and reacting the reaction mixture to form a reaction product mixture comprising compound 093a according to the following reaction scheme

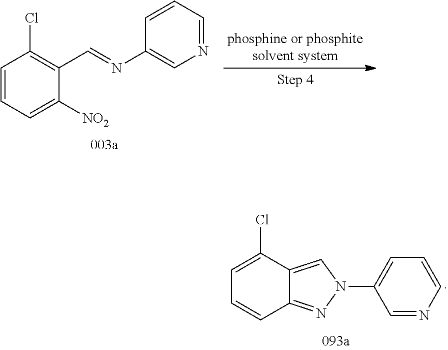 [SMILES: O=[N+]([O-])c1cccc(Cl)c1/C=N/c1cccnc1 ; Clc1cccc2nn(-c3cccnc3)cc12]

In some aspects, the solvent system comprises or predominantly comprises a polar protic solvent or a nonpolar solvent. In some such aspects, the solvent system comprises or predominantly comprises a C₁₋₄ alcohol. In one such aspect, the solvent system comprises or predominantly comprises isopropyl alcohol. In some aspects, the solvent system comprises or predominantly comprises toluene.

In some aspects, the phosphine or phosphite is trimethyl phosphite, triethyl phosphite, triisopropyl phosphite, triphenylphosphite, trimethyl phosphine, triethyl phosphine, tributyl phosphine, or triphenyl phosphine.

In some aspects, the reaction is run at reflux.

In some aspects, any of compounds 181a, 050, 378, 003a, and 093a are optionally isolated from the reaction product mixture. In some aspects, any two sequential reactions, or all of the sequential reactions from compound 339 to compound 093a are done in a telescopic pot scheme.

Some aspects of the disclosure are directed to a process for the preparation of compound 093a, the process comprising seven steps.

Step 1 comprises forming a reaction mixture comprising compound 150 (2,6-dichlorobenzaldehyde), an acid, and a solvent system, and reacting the reaction mixture to form a reaction product mixture comprising compound 086 (1-(2,6-dichlorophenyl)-N-(pyridin-3-yl) methanimine) according to the following

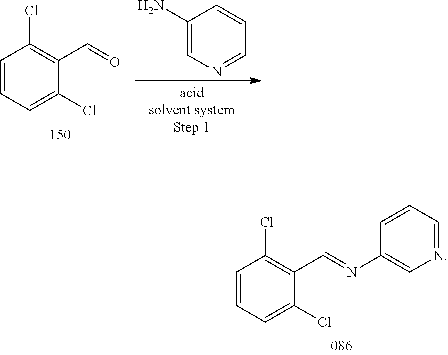 [SMILES: O=Cc1c(Cl)cccc1Cl ; Nc1cccnc1 ; Clc1cccc(Cl)c1/C=N/c1cccnc1]

In some aspects, the acid is p-toluenesulfonic acid.

In some aspects, the solvent system comprises or predominantly comprises at least one non-polar solvent. In some such aspects, the solvent system comprises or predominantly comprises toluene. Step 2 comprises forming a reaction mixture comprising compound 086, a reducing agent, and a solvent system, and reacting the reaction mixture to form a reaction product mixture comprising compound 084 (N-(2,6-dichlorobenzyl)pyridin-3-amine) according to the following reaction scheme

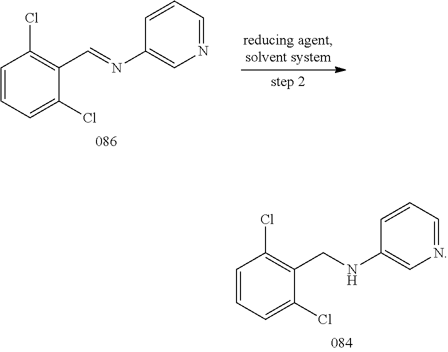 [SMILES: Clc1cccc(Cl)c1/C=N/c1cccnc1 ; Clc1cccc(Cl)c1CNc1cccnc1]

In some aspects, the reducing agent is selected from sodium borohydride and sodium cyanoborohydride.

In some aspects, the solvent system comprises or predominantly comprises a polar protic solvent, the solvent system comprises or predominantly comprises a C₁₋₄ alcohol, or the solvent system comprises or predominantly comprises methanol.

Step 3 comprises forming a reaction mixture comprising compound 084, a reagent for converting an amine moiety to a nitrosamine moiety, an acid, and a solvent system, and reacting the reaction mixture to form a reaction product mixture comprising compound 085 (N-(2,6-dichlorobenzyl)-N-(pyridin-3-yl) nitrous amide) according to the following reaction scheme

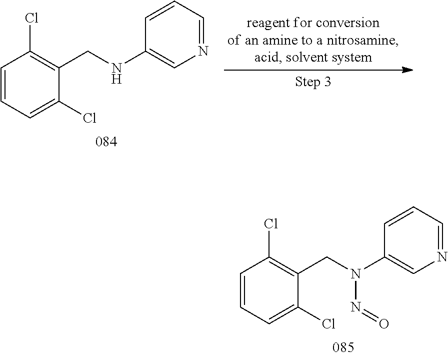 [SMILES: Clc1cccc(Cl)c1CNc1cccnc1 ; O=NN(Cc1c(Cl)cccc1Cl)c1cccnc1]

In some aspects, the reagent for conversion of an amine to a nitrosamine is a nitrite. In some such aspects, the reagent for conversion of an amine to a nitrosamine is sodium nitrite.

In some aspects, the acid is an organic acid. In some such aspects, the acid is p-toluenesulfonic acid.

In some aspects, the solvent system comprises or predominantly comprises at least one polar aprotic solvent. In some such aspects, the solvent system comprises or predominantly comprises DCM.

Step 4 comprises: (a) forming a reaction mixture comprising compound 085, a reductant, a base, and a solvent system, and reacting the reaction mixture and (b) acidification to form a reaction product mixture comprising compound 048 (1-[(2,6-dichlorophenyl)methyl]-1-(3-pyridyl) hydrazine salt) according to the following reaction scheme

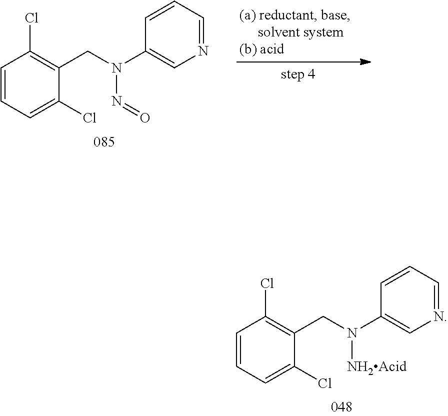

In some aspects, the reductant is thiourea dioxide.

In some aspects, the base is an inorganic base. In some such aspects, the base is an alkali metal hydroxide. In some such aspects, the base is NaOH or KOH.

In some aspects, the solvent system comprises or predominantly comprises at least one polar protic solvent. In some such aspects, the solvent system comprises or predominantly comprises water.

In some aspects, the acid is an inorganic acid. In some aspects the acid is HCl. In some aspects, the acid is HCl in isopropanol and compound 048 is (3-(1-(2,6-dichlorobenzyl) hydrazineyl)pyridine*HCl).

Step 5 comprises forming a reaction mixture comprising compound 048, acetic anhydride, a base, and a solvent system, and reacting the reaction mixture to form a reaction product mixture comprising compound 083 (N′-(2,6-dichlorobenzyl)-N′-(pyridin-3-yl) acetohydrazide) according to the following reaction scheme.

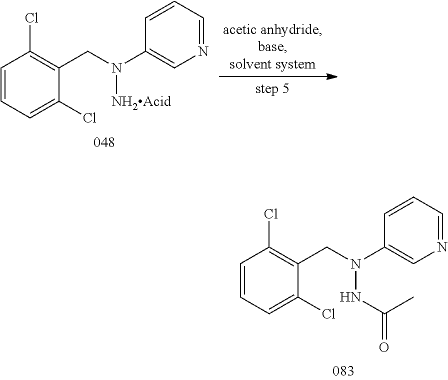

In some aspects, the base is an organic base. In some such aspects, the base is triethylamine.

In some aspects, the solvent system comprises or predominantly comprises at least one polar aprotic solvent. In some such aspects, the solvent system comprises or predominantly comprises dichloromethane.

Step 6 comprises forming a reaction mixture comprising compound 083, a ligand, a transition metal catalyst, a base, and a solvent system, and reacting the reaction mixture to form a reaction product mixture comprising compound 082 (1-[4-chloro-2-(3-pyridyl)-3H-indazol-1-yl]ethanone) according to the following reaction scheme.

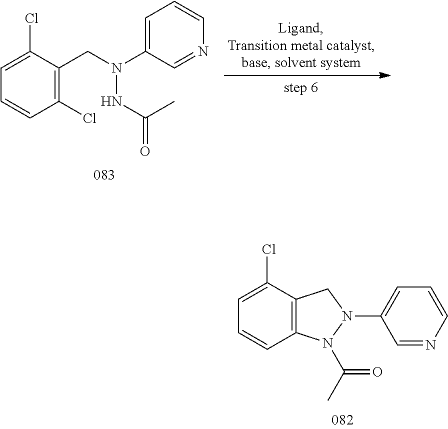 [SMILES: CC(=O)NN(Cc1c(Cl)cccc1Cl)c1cccnc1 ; CC(=O)N1c2cccc(Cl)c2CN1c1cccnc1]

In some aspects, the ligand is ethylene diamine or trans-dimethylcyclohexyl-1,2-diamine.

In some aspects, the transition metal catalyst is selected from Group VIII, Group IX, Group X, or Group XI metals. In some aspects, the transition metal catalyst is selected from a Pd catalyst and a Cu catalyst. In some aspects, the transition metal catalyst is CuI.

In some aspects, the base is an inorganic base. In some such aspects, the base is K₃PO₄.

In some aspects, the solvent system comprises or predominantly comprises at least one non-polar solvent. In one aspect, the solvent system comprises predominantly comprises dioxane. In another aspect, the solvent system predominantly comprises toluene.

Step 7 comprises forming a reaction mixture comprising compound 082, a base, and a solvent system, and reacting the reaction mixture to form a reaction product mixture comprising compound 093a according to the following reaction scheme

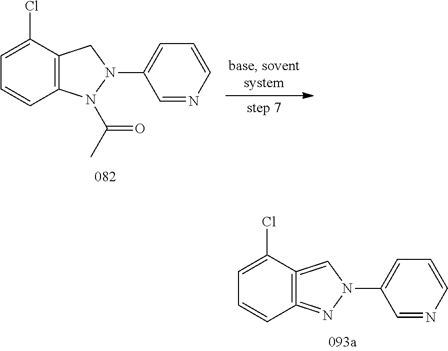

In some aspects, the base is an inorganic base. In some such aspects, the base is a weak inorganic base. In some aspects, the base is a carbonate. In some aspects, the base is selected from sodium carbonate, sodium bicarbonate, potassium carbonate, and potassium bicarbonate.

In some aspects, the solvent system comprises or predominantly comprises a polar protic solvent. In some such aspects, the solvent system comprises or predominantly comprises a C₁₋₄ alcohol. In some such aspects, the solvent system comprises or predominantly comprises methanol.

In some aspects, reactions steps 6 and 7 are done sequentially in a single vessel.

Some aspects of the disclosure are directed to a process for the preparation of compound 093a, the process comprising three steps.

In the first step, a reaction mixture solution is formed comprising compound 150 (2,6-dichlorobenzaldehyde), acetohydrazine, a solvent system, and an organic acid, and the reaction mixture is reacted to form a reaction product mixture comprising compound 197 (N-[(2,6-dichlorophenyl)methyleneamino]acetamide) according to the following reaction scheme

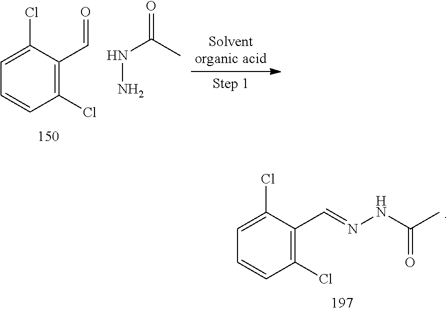

In some aspects, the organic acid is p-toluenesulfonic acid. In some aspects, the solvent system comprises at least one nonpolar solvent. In some such aspects, the solvent system comprises toluene.

In some aspects, the reaction is run at reflux with azeotropic distillation of water to drive the condensation process.

In the second step, a reaction mixture is formed comprising compound 197, a reducing agent, and a solvent system, and the reaction mixture is reacted to form a reaction product mixture comprising compound 040 (N′-[(2,6-dichlorophenyl)methyl]acetohydrazide) according to the following reaction scheme

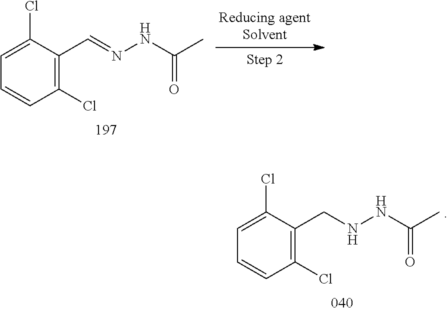

In some aspects, the reducing agent is magnesium or a borohydride. In some such aspects, the reducing agent is selected from sodium borohydride, sodium cyanoborohydride, and magnesium.

In some aspects, the solvent system comprises at least one alcohol, at least one carboxylic acid, or a combination thereof. In some such aspects, the solvent system comprises methanol, ethanol, acetic acid, or a combination thereof.

In the third step, a reaction mixture is formed comprising compound 040, 3-bromopyridine, a ligand, a transition metal catalyst, and a solvent system, and the reaction mixture is reacted to form a reaction product mixture comprising compound 093a according to the following reaction scheme

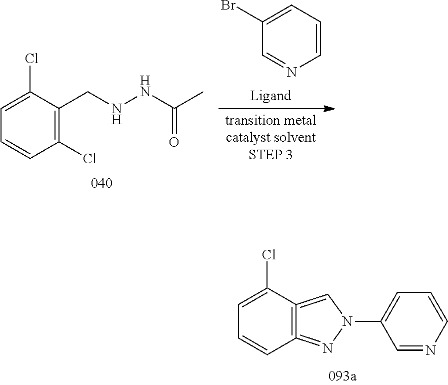

In some aspects, the ligand is a diamine ligand. In some such aspects the ligand is selected from trans-N,N′-dimethylcyclohexyldiamine and N,N′-dimethylethylenediamine.

In some aspects, the transition metal catalyst is a copper catalyst. In some such aspects, the transition metal catalyst is CuI.

In some aspects, the solvent system comprises a nonpolar solvent. In some such aspects, the solvent system comprises toluene.

Some aspects of the disclosure are directed to a process for the preparation of compound 093a or 093b (4-chloro-2-(3-pyridyl) indazole or 4-bromo-2-(3-pyridyl) indazole), the process comprising two steps.

In the first step, a reaction mixture solution is formed comprising compound 181a or 181b (2-(chloromethyl)-1-chloro-3-nitro-benzene or 2-(bromomethyl)-1-chloro-3-nitro-benzene), compound 520 (3-aminopyridinium salt), and a solvent system, and the reaction mixture is reacted to form a reaction product mixture comprising compound 182a or 182b (N-[(2-chloro-6-nitro-phenyl)methyl]pyridin-3-amine or N-[(2-bromo-6-nitro-phenyl)methyl]pyridin-3-amine) according to the following reaction scheme.

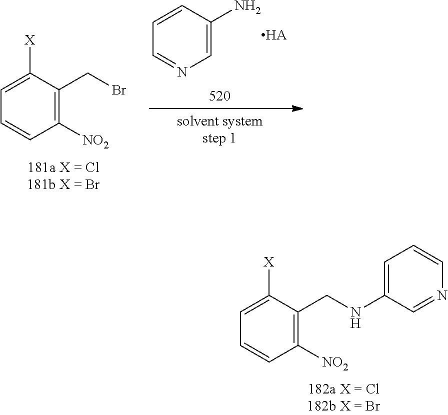

In some aspects, compound 520 is a hydrochloride, hydrobromide, sulfate, bisulfate, methanesulfonate, or p-toluenesulfonate salt. In some such aspects compound 520 optionally is prepared in situ or prepared and isolated prior to use.

In some aspects, the solvent system is comprises or predominately comprises a polar aprotic solvent. In some such aspects the solvent system comprises acetonitrile, benzonitrile, sulfolane, or a combination thereof. In some aspects, the solvent system comprises a mixture of a non polar solvent and a polar protic solvent. In some such aspects, the solvent system is toluene and water or xylenes and water. Optionally, a phase transfer catalyst (PTC) is also included. In some such step 3 aspects, the PTC is a quaternary ammonium salt. In some such aspects, the PTC is tetrabutylammonium bromide. In some such aspects, the PTC is used in a catalytic amount.

In some aspects, the 182a or 182b product is isolated as the anilinium bromide salt, the di-bromide salt, or a salt mixture of HBr and methanesulfonic acid or p-toluenesulfonic acid. If 182a or 182b is treated with a base after the reaction, it is isolated as the free base.

In the second step, a reaction mixture solution is formed comprising 182a or 182b, reductant, a base, and a solvent system, and the reaction mixture is reacted to form a reaction product mixture comprising compound 093a or 093b according to the following reaction scheme

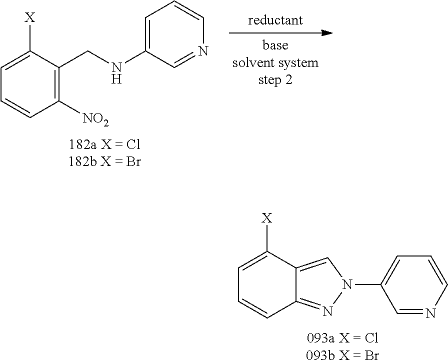

In some aspects, the reductant is a reducing agent. In some aspects, the reductant is selected from zinc, iron, or titanium tetrachloride,

In some aspects, the base is an inorganic base. In some such aspects the base is a hydroxide base. In some such aspects, the base is selected from sodium hydroxide, potassium hydroxide, or cesium hydroxide. When titanium tetrachloride is the reductant the base is triethylamine

In some aspects, the solvent system is comprised of a mixture of non-polar or polar solvent and a polar protic solvent. In some aspects, the solvent system is selected from water and 1,4-dioxane, water and isopropanol, water and tetrahydrofuran, water and toluene, water and xylenes, water and N-methylpyrrolidone, water and sulfolane, or water and dimethylacetamide In some aspects, the solvent system comprises or predominately comprises a polar protic solvent. In some such aspects, the solvent system is water.

In some aspects, any of compound 182a, 182b, 093a, or 093b are optionally isolated from the reaction product mixture. In some aspects, any two sequential reactions, or all of the sequential reaction from compound 181a or 181b to 093a or 093b are done in a telescopic pot scheme.

Some aspects of the disclosure are directed to an alternate process for the preparation of compound 182a or 182b (N-[(2-chloro-6-nitro-phenyl)methyl]pyridin-3-amine or N-[(2-bromo-6-nitro-phenyl)methyl]pyridin-3-amine). The process comprises forming a reaction mixture comprising compound 003a or 003b (1-(2-chloro-6-nitro-phenyl)-N-(3-pyridyl) methanimine or 1-(2-bromo-6-nitro-phenyl)-N-(3-pyridyl) methanimine), a reducing agent, and a solvent system and reacting the reaction mixture to form a reaction product mixture comprising compound 182a or 182b according to the following reaction scheme.

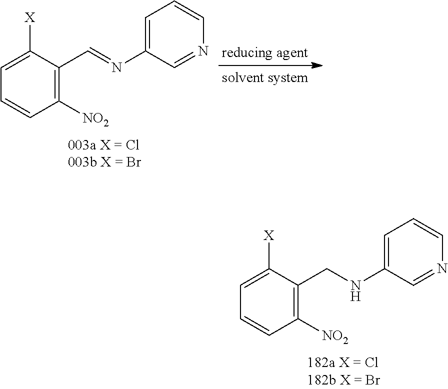

In some aspects, the reducing agent is magnesium. In some such aspects the reducing agent is a borohydride. In some such aspects the reducing agent is selected from sodium borohydride or sodium cyanoborohydride.

In some aspects, the solvent system comprises at least one C₁₋₄ alcohol, a carboxylic acid, or a combination thereof. In some such aspects, the solvent system comprises methanol, ethanol, acetic acid, or a combination thereof.

Some aspects of the disclosure are directed to a process for the preparation of compound 093a or 093b (4-chloro-2-(3-pyridyl) indazole or 4-bromo-2-(3-pyridyl) indazole) according to a scheme comprising three steps.

Step 1 for preparing compound 093a or 093b comprises forming a reaction mixture comprising compound 114a or 114b (3-chloro-2-methyl-aniline or 3-bromo-2-methyl-aniline), an oxidizing agent, and a solvent system, and reacting the reaction mixture to form a reaction product mixture comprising compound 115a or 115b (3-chloro-2-methyl-nitrosobenzene or 3-bromo-2-methyl-3-nitroso-benzene) according to the following reaction scheme.

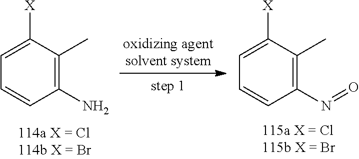

In some In some step 1 aspects, the oxidizing agent is potassium peroxymonosulfate, sodium perborate, sodium percarbonate, hydrogen peroxide, peracetic acid, or 3-chloroperbenzoic acid.

In some step 1 aspects, the solvent system comprises or predominately comprises at least one polar aprotic solvent and at least one polar protic solvent. In some such aspects, the solvent system comprises or predominately comprises dichloromethane and water or dichloroethane and water.

In some step 1 aspects, compound 115a or 115b may be optionally isolated from the reaction product mixture.

Step 2 for preparing compound 093a or 093b comprises forming a reaction mixture comprising compound 115a or 115b, a source of bromine or a source of chlorine, optionally a radical initiator, and a solvent system, and reacting the reaction mixture under photochemical conditions to form a reaction product mixture comprising compound 116a-d (1-chloro-2-(chloromethyl)-3-nitroso-benzene, 1-chloro-2-(bromomethyl)-3-nitroso-benzene, 1-bromo-2-(chloromethyl)-3-nitroso-benzene, or 1-bromo-2-(bromomethyl)-3-nitroso-benzene) according to the following reaction scheme.

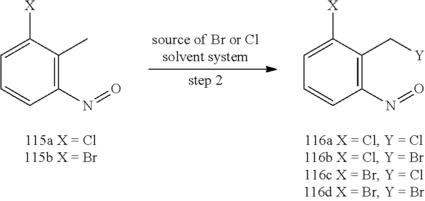

In some step 2 aspects, the source of Br is N-bromosuccinimide, Br₂, or HBr and hydrogen peroxide. In some step 2 aspects, the source of Cl is N-chlorosuccinimide, trichloroisocyanuric acid, Cl₂, or HCl and hydrogen peroxide.

In some step 2 aspects, the photochemical conditions relate to promotion of the reaction using a source of light. In some such step 2 aspects, the photochemical conditions promote the reaction using visible light.

In some step 2 aspects, a photosensitive or thermally activated radical initiator is used. In some such aspects the radical initiator is azobisisobutyronitrile.

In some step 2 aspects, the solvent system comprises or predominantly comprises at least one polar aprotic solvent and at least one polar protic solvent. In some such aspects, the solvent system comprises or predominantly comprises dichloromethane and water or dichloroethane and water.

In some step 2 aspects, compound 116a-d may be optionally isolated from the reaction product mixture.

Step 3 of the first scheme for preparing compound 093a or 093b comprises forming a reaction mixture comprising compound 116a-d, compound 520 (3-aminopyridinium salt), and a solvent system, and reacting the reaction mixture to form a reaction product mixture comprising compound 093a 093b according to the following reaction scheme.

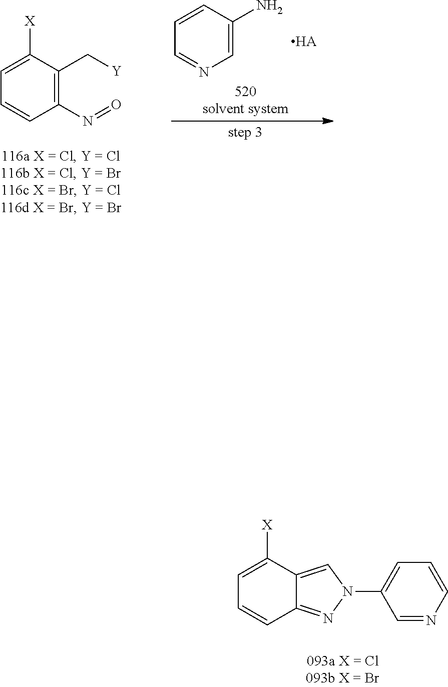

In some step 3 aspects, compound 520 is a hydrochloride, hydrobromide, hydroiodide, sulfate, bisulfate, formic, methanesulfonate, or para-toluenesulfonate salt. In some such step 3 aspects, compound 520 is optionally prepared in situ or prepared and isolated prior to use.

In some step 3 aspects, the solvent system comprises or predominantly comprises a polar aprotic solvent. In some such aspects, the solvent system comprises or predominantly comprises dichloromethane, dichloroethane, or acetonitrile. In some step 3 aspects, the solvent system comprises or predominantly comprises at least one aprotic solvent and at least one polar protic solvent. In some such aspects, the solvent system comprises or predominantly comprises dichloromethane and water, dichloroethane and water, acetonitrile and water, toluene and water, or combinations thereof.

In some step 3 aspects, a phase transfer catalyst is used. In some such step 3 aspects, the phase transfer catalyst is a quaternary ammonium salt. In some such step 3 aspects, the phase transfer catalyst is tetrabutylammonium chloride, tetrabutylammonium bromide, or tetrabutylammonium iodide.

In some step 3 aspects, compound 093a or 093b may be optionally isolated from the reaction product mixture.

In some aspects, any of compound 115a or 115 b, 116a-d, or 093a or 093b are optionally isolated from the reaction product mixture. In some aspects, any two sequential reactions, or all of the sequential reaction from compound 114a or 114b to 093a or 093b are done in a telescopic pot scheme.

Some aspects of the disclosure are directed to a first process for the preparation of compound 061 (2-(pyridin-3-yl)-2H-indazole-4-carboxylic acid). The process comprises forming a reaction mixture comprising compound 093a or 93b (4-chloro-2-(3-pyridyl) indazole or 4-bromo-2-(3-pyridyl) indazole), CO, a catalyst, a ligand, a base, and a solvent system, and reacting the reaction mixture to form a reaction product mixture comprising compound 061 according to the following reaction scheme according to the following reaction scheme

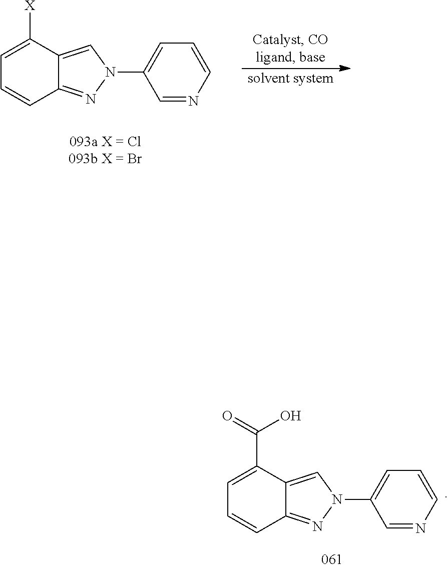

In some aspects, the catalyst is selected from a transition metal catalyst, In some aspects, the catalyst is selected from a palladium catalyst, a nickel catalyst, a platinum catalyst, and a copper catalyst. In some aspects, the catalyst is selected from a palladium catalyst. In some such aspects, the catalyst is selected from palladium on carbon or palladium acetate.

In some aspects, the ligand is a diphosphine ligand. In some such aspects, the ligand is 1,3-bis(dicyclohexylphosphium)propane bis(tetrafluoroborate), 1,3-bis(dicyclohexylphosphino)propane, 1,3-bis(diphenylphosphino)propane, or 1,3-bis(diphenylphoshonium) bis(tetrafluoroborate). In one such aspect, the ligand is 1,3-bis(dicyclohexylphosphino)propane bis(tetrafluoroborate).

In some aspects, the base is an inorganic base. In some such aspects, the base is a carbonate. In some such aspects, the base is selected from sodium carbonate, sodium bicarbonate, potassium carbonate, and potassium bicarbonate. In some such aspects, the base is potassium carbonate.

In some aspects, the reaction atmosphere comprises or predominately comprises a mixture CO and N₂. In some aspects, the reaction atmosphere comprises or predominately comprises CO. In some aspects, the reaction temperature is at least 100° C.

In some aspects, the solvent system comprises or predominantly comprises a polar aprotic solvent. In some aspects, the solvent system comprises or predominantly comprises dimethyl sulfoxide. In some aspects, the solvent system further comprises water. In one such aspect, the solvent system comprises dimethyl sulfoxide and water.

Some aspects of the disclosure are directed to a second process for the preparation of compound 061 (2-(3-pyridyl)indazole-4-carboxylic acid). The process comprises forming a reaction mixture comprising compound 038 (methyl 2-(3-pyridyl)indazole-4-carboxylate), a base, and a solvent system, and reacting the reaction mixture to form a reaction product mixture comprising compound 061 according to the following reaction scheme

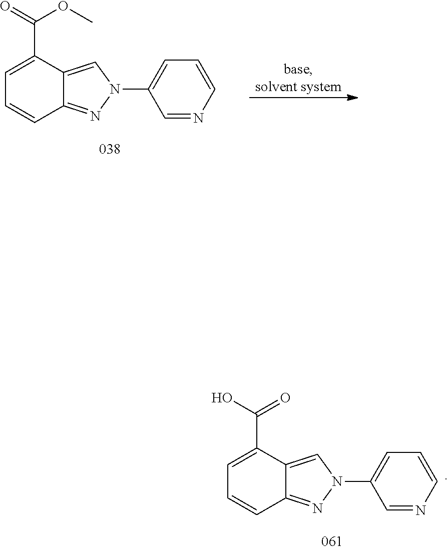 [SMILES: COC(=O)c1cccc2nn(-c3cccnc3)cc12 ; O=C(O)c1cccc2nn(-c3cccnc3)cc12]

In some aspects, the base is an alkali metal hydroxide. In some such aspects, the base is potassium hydroxide or sodium hydroxide. In some such aspect, the base is sodium hydroxide.

In some aspects, the solvent system comprises or predominantly comprises water and a polar organic solvent. In one aspect, the solvent system comprises or predominantly comprises water and at least one solvent selected from acetone, acetonitrile, isopropanol, methanol, ethanol, dimethylsulfoxide, dimethylacetamide, dimethylformamide, N-methylpyrrolidone, and combinations thereof.

Some aspects of the disclosure are directed to a process for the preparation of compound 038 (methyl 2-(3-pyridyl) indazole-4-carboxylate according to a first, second, or third scheme

The first scheme for preparing compound 038 comprises three steps.

Step 1 of the first scheme for preparing compound 038 comprises forming a reaction mixture comprising compound 400 (methyl 3-amino-2-methylbenzoate), an oxidizing agent, and a solvent system, and reacting the reaction mixture to form a reaction product mixture comprising compound 500 (methyl 2-methyl-3-nitrosobenzoate) according to the following reaction scheme

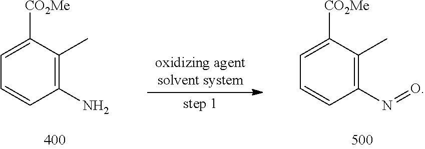

In some step 1 aspects, the oxidizing agent is potassium peroxymonosulfate, sodium perborate, sodium percarbonate, hydrogen peroxide, peracetic acid, or 3-chloroperbenzoic acid.

In some step 1 aspects, the solvent system comprises or predominantly comprises at least one polar aprotic solvent and at least one polar protic solvent. In some such aspects, the solvent system comprises or predominantly comprises dichloromethane and water or dichloroethane and water.

In some step 1 aspects, compound 500 may be optionally isolated from the reaction product mixture.

Step 2 of the first scheme for preparing compound 038 comprises forming a reaction mixture comprising compound 500, a source of bromine or a source of chlorine, optionally a radical initiator, and a solvent system, and reacting the reaction mixture under photochemical conditions to form a reaction product mixture comprising compound 510a or 510b (methyl 2-(bromomethyl)-3-nitrosobenzoate or methyl 2-(chloromethyl)-3-nitrosobenzoate) according to the following reaction scheme

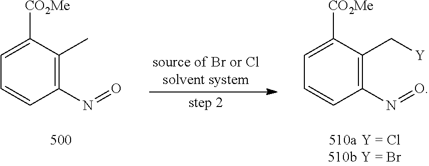

In some step 2 aspects, the source of Br is N-bromosuccinimide, Br₂, or HBr and hydrogen peroxide. In some step 2 aspects, the source of Cl is N-chlorosuccinimide, trichloroisocyanuric acid, Cl₂, or HCl and hydrogen peroxide.

In some step 2 aspects, the photochemical conditions relate to promotion of the reaction by a source of light.

In some step 2 aspects, a photosensitive or thermally activated radical initiator may be used optionally. In some such aspects, the radical initiator is azobisisobutyronitrile.

In some step 2 aspects, the solvent system comprises or predominantly comprises at least one polar aprotic solvent and at least one polar protic solvent. In some such aspects, the solvent system comprises or predominantly comprises dichloromethane and water or dichloroethane and water.

In some step 2 aspects, compound 510a or 510b may be optionally isolated from the reaction product mixture.

Step 3 of the first scheme for preparing compound 038 comprises forming a reaction mixture comprising compound 510a or 510b, compound 520 (3-aminopyridinium salt), and a solvent system, and reacting the reaction mixture to form a reaction product mixture comprising compound 038 according to the following reaction scheme.

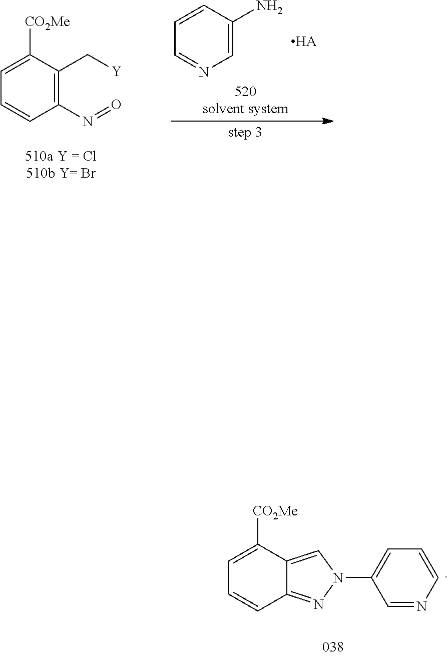

In some step 3 aspects, compound 520 is a hydrochloride, or hydrobromide, hydroiodide, sulfate, bisulfate, methanesulfonate, or p-toluenesulfonate salt. In some aspects, compound 520 is a hydrochloride salt. In some such step 3 aspects, the compound 520 optionally is prepared in situ or prepared and isolated prior to use.

In some step 3 aspects, the solvent system comprises or predominantly comprises a polar aprotic solvent. In some such aspects, the solvent system comprises or predominantly comprises dichloromethane, dichloroethane, or acetonitrile. In some step 3 aspects, the solvent system comprises or predominantly comprises at least one aprotic solvent and at least one polar protic solvent. In some such aspects, the solvent system comprises or predominantly comprises dichloromethane and water, dichloroethane and water, acetonitrile and water, or toluene and water.

In some step 3 aspects, a phase transfer catalyst is used. In some such step 3 aspects, the phase transfer catalyst is a quaternary ammonium salt. In some such step 3 aspects, the phase transfer catalyst is tetrabutylammonium chloride or tetrabutylammonium bromide.

In some step 3 aspects, compound 038 may be optionally isolated from the reaction product mixture.

In some aspects, any of compound 500, 510a or 510b, or 038 are optionally isolated from the reaction product mixture. In some aspects, any two sequential reactions, or all of the sequential reaction from compound 400 to 038 are done in a telescopic pot scheme.

The second scheme for preparing compound 038 comprises three steps.

Step 1 of the second scheme for preparing compound 038 comprises forming a reaction mixture comprising compound 400 where “Protec” refers to an amine protecting group, a source of bromine or a source of chlorine, and a solvent system, and reacting the reaction mixture by exposure to a source of light to form a reaction product mixture comprising compound 410 (methyl 3-amino-2-(bromomethyl)benzoate or methyl 3-amino-2-(chloromethyl)benzoate) according to the following reaction scheme

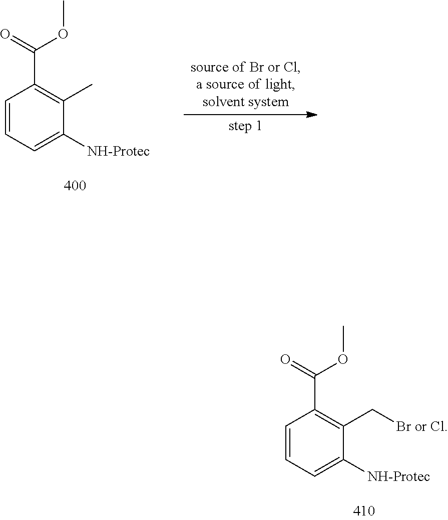

In some step 1 aspects, the source of Br is Br₂ and hydrogen peroxide. In some step 1 aspects, the source of Cl is Cl₂. In some step 1 aspects, the source of Cl is Cl₂. and hydrogen peroxide.

In some aspects, the protected amine is acetamide of the structure —NHC (O) CH₃.

In some step 1 aspects, the solvent system comprises or

predominantly comprises at least one polar aprotic solvent and at least one polar protic solvent. In some such aspects, the solvent system comprises or predominantly comprises dichloromethane and water.

In some step 1 aspects, compound 410 may be optionally isolated from the reaction product mixture.

The protecting group is removed from compound 400 prior to formation of compound 430 in step 2. When the protected amine is acetamide, deprotection by secondary amine diacylation may suitably be done by reacting 410 with a weak inorganic base or organic base in water, polar protic solvent, or polar nonprotic solvent.

Step 2 of the second scheme for preparing compound 038 comprises forming a reaction mixture comprising compound 410, compound 420, an acid, and a solvent system, and reacting the reaction mixture to form a reaction product mixture comprising compound 430 ((E)-2-bromo-6-(pyridin-3-yldiazenyl)benzyl acetate or (E)-2-chloro-6-(pyridin-3-yldiazenyl)benzyl acetate) according to the following

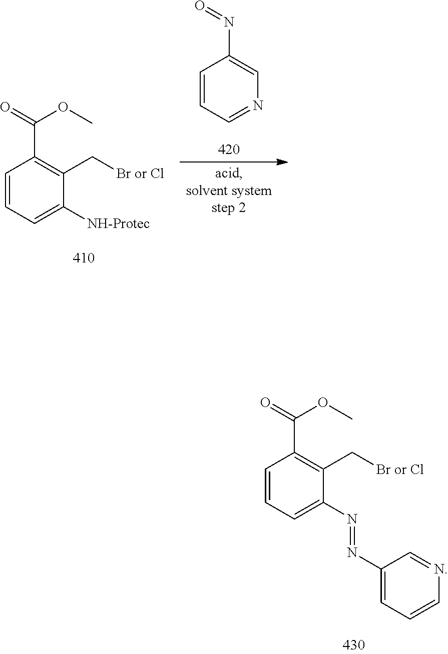

In some step 2 aspects, the acid is an organic acid. In some such aspects, the acid is acetic acid.

In some step 2 aspects, the solvent system comprises or predominantly comprises acetic acid.

In some step 2 aspects, compound 430 may be optionally isolated from the reaction product mixture.

Step 3 of the second scheme for preparing compound 038 comprises forming a reaction mixture comprising compound 430, a strong acid, and a solvent system, and reacting the reaction mixture to form a reaction product mixture comprising compound 038 according to the following reaction scheme

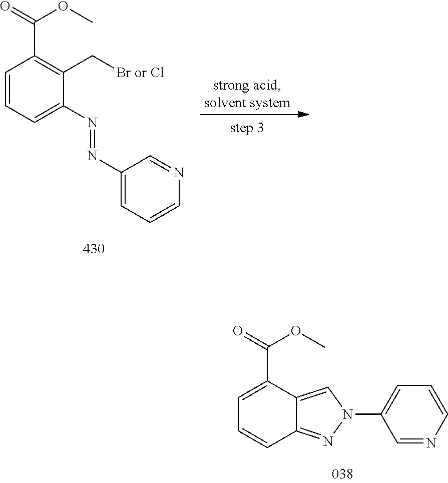

In some step 3 aspects, the strong acid is an inorganic acid. In some such aspects, the acid is a mineral acid. In some such aspects, the acid is HCl.

In some step 3 aspects, the solvent system comprises or predominantly comprises at least one polar protic solvent. In some such aspects, the solvent system comprises or predominantly comprises water, methanol, ethanol, isopropanol, acetic acid, or a combination thereof.

In some step 3 aspects, compound 038 may be optionally isolated from the reaction product mixture.

The third scheme for preparing 038 (methyl 2-(3-pyridyl) indazole-4-carboxylate) comprises one step. The process comprises forming a reaction mixture comprising compound 093a or 093b (4-chloro-2-(3-pyridyl) indazole or 4-bromo-2-(3-pyridyl) indazole), a catalyst, a ligand, CO, a base, and a solvent system, and reacting the reaction mixture to form a reaction product mixture comprising compound 038 according to the following reaction scheme.

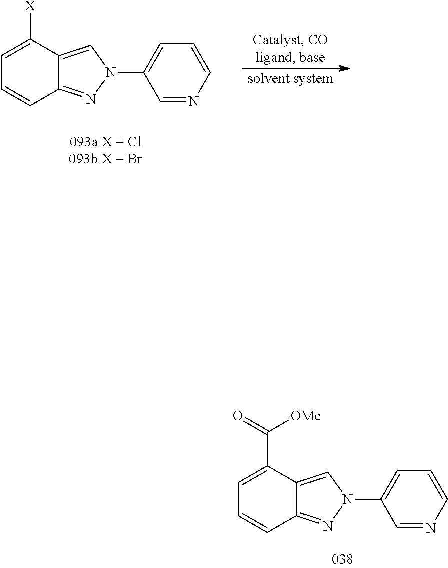

In some aspects, the catalyst is a transition metal catalyst. In some such aspects, the catalyst selected from a palladium catalyst, a nickel catalyst, or a copper catalyst. In some such aspects the catalyst is a palladium on carbon or palladium (II) acetate.

In some aspects, the ligand is a diphosphine ligand. In some such aspects the ligand is selected from 1,3-bis(dicyclohexylphosphino) propane, 1,3-bis(dicyclohexylphosphonium) propane bis(tetrafluoroborate), 1,3-bis(diphenylphosphino) propane, or 4,5-bis(diphenylphosphino)-9,9-dimethylxanthene.

In some aspects, the base is one or more weak inorganic base. In some such aspects, the base is a carbonate or phosphate. In some such aspects, the base is selected from sodium carbonate, sodium bicarbonate, potassium carbonate, potassium bicarbonate, sodium phosphate tribasic, and potassium phosphate tribasic or combinations thereof. In some aspects, the base is an organic base. In some such aspects, the base is triethylamine.

In some aspects, the solvent system comprises or predominantly comprises a nonpolar solvent and methanol. In some such aspects, the solvent system comprises or predominantly comprises xylenes and methanol, o-xylene and methanol, or toluene and methanol. In some aspects the solvent system comprises or predominantly comprises a polar aprotic solvent and methanol. In some such aspects, the solvent system comprises DMSO and methanol, DMF and methanol, NMP and methanol.

In some aspects, the reaction atmosphere is comprised of a mixture of CO and N₂. In some aspects, the reaction atmosphere is comprised of predominantly CO. In some aspects, the reaction temperature is at least 100° C.

In some aspects, the generated compound 038 can be converted in situ to compound 061 (2-(3-pyridyl) indazole-4-carboxylic acid) using hydrolysis under basic or acidic conditions. In some such aspects, the hydrolysis is conducted using alkali metal hydroxide bases. In some such aspects the hydrolysis is conducted using sodium hydroxide or potassium hydroxide.

Another aspect of the disclosure is directed to an alternative process for the preparation of compound 038 (methyl 2-(3-pyridyl) indazole-4-carboxylate). The process comprises forming a reaction mixture comprising compound 061 (2-(3-pyridyl) indazole-4-carboxylic acid), an acid, optionally an additive, and a solvent system containing methanol, and reacting the reaction mixture to form a reaction product mixture comprising compound 038 according to the following reaction scheme

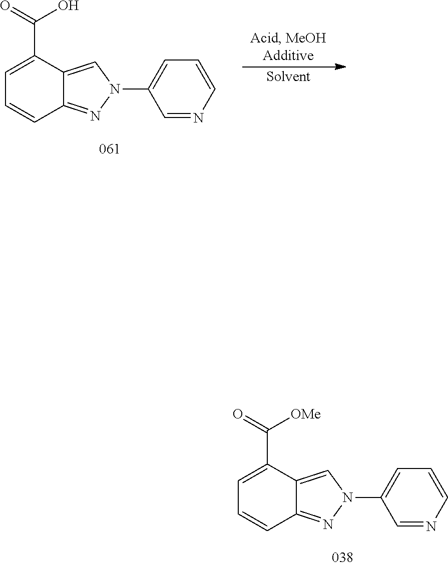 [SMILES: O=C(O)c1cccc2nn(-c3cccnc3)cc12 ; COC(=O)c1cccc2nn(-c3cccnc3)cc12]

In some aspects, the acid is an inorganic acid. In some such aspects, the acid is selected from sulfuric acid or hydrochloric acid. In some aspects, the acid is an organic acid. In some such aspects the acid is p-toluene sulphonic acid.

In some aspects the additive is a dehydrating agent such as molecular sieves.

In some aspects, the solvent system comprises or predominantly comprises a nonpolar solvent and methanol. In some such aspects, the solvent system comprises or predominantly comprises methanol, toluene, hexanes, or combinations thereof.

Some aspects of the disclosure are directed to a process for the preparation of compound 092 ((N-(1-methylcyclopropyl)-2-(3-pyridinyl)-2H-indazole-4-carboxamide) by a two-step process.

Step 1 for preparing compound 092 (N-(1-methylcyclopropyl)-2-(3-pyridyl) indazole-4-carboxamide) comprises forming a reaction mixture comprising compound 061 (2-(pyridin-3-yl)-2H-indazole-4-carboxylic acid), a chlorinating reagent, and a solvent system, and reacting the reaction mixture to form a reaction product mixture comprising compound 930 (2-(3-pyridyl) indazole-4-carbonyl chloride) HCl salt according to the following reaction scheme

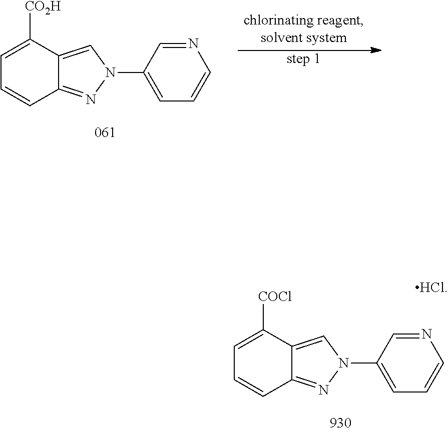 [SMILES: O=C(O)c1cccc2nn(-c3cccnc3)cc12 ; "·HCl." ; O=C(Cl)c1cccc2nn(-c3cccnc3)cc12]

In some step 1 aspects, the solvent system comprises or predominantly comprises a nonpolar solvent, a polar aprotic solvent, or a combination thereof. In some such aspects, the solvent system comprises or predominantly comprises toluene and N,N-dimethylformamide. In some such aspects, the solvent system comprises or predominantly comprises toluene. In some such aspects, the solvent system comprises or predominantly comprises acetonitrile and N,N-dimethylformamide. In some such aspects, the solvent system comprises or predominantly comprises acetonitrile.

In some step 1 aspects, the reaction mixture optionally further comprises a catalyst. A suitable catalyst can be selected from among N,N-dimethylformamide, 4-dimethylaminopyridine, triethylamine, N-methyl-2-pyrrolidone, N-methylformanilide, N-formylpyridine, and pyridine. In some such aspects, the catalyst is N,N-dimethylformamide. In some such aspects, the catalyst is pyridine.

In some aspects, the chlorinating reagent may be selected from thionyl chloride, oxalyl chloride, phosphorous oxychloride, cyanuric chloride, diphosgene, triphosgene, and phosgene. The chlorination reagent is preferably in stoichiometric excess compared to compound 930.

In some step 1 aspects, compound 930 may be optionally isolated from the reaction product mixture.

Step 2 comprises forming a reaction mixture comprising compound 930, compound 070 (1-methylcyclopropanamine), a base, and a solvent system, and reacting the reaction mixture to form a reaction product mixture comprising compound 092 according to the following reaction scheme

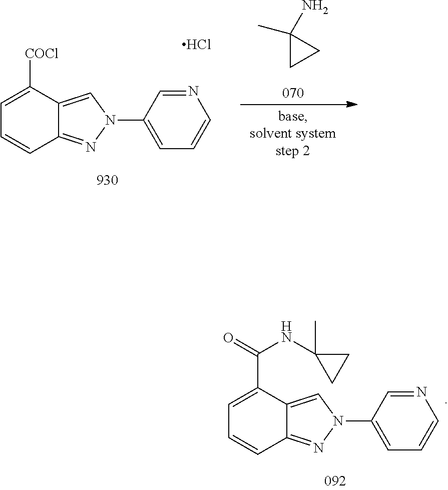

In some step 2 aspects, the solvent system comprises or predominantly comprises a nonpolar solvent, a polar aprotic solvent, or a combination thereof. In some step 2 aspects, the solvent system can be selected from among toluene, xylene, N,N-dimethylformamide, N-methyl-2-pyrrolidone, acetonitrile, dimethylacetamide, isopropyl acetate, tetrahydrofuran, dichloromethane, pyridine and sulfolane. In some such aspects, the solvent system comprises or predominantly comprises a polar solvent. In some such aspects, the solvent system comprises one or more polar solvents. In some such aspects, the solvent system comprises or predominantly comprises acetonitrile. In some such aspects, the solvent system comprises acetonitrile and N-methyl-2-pyrrolidone. In some such aspects, the solvent system comprises or predominantly comprises a nonpolar solvent. In some such aspects, the solvent system comprises or predominantly comprises toluene. In some such aspects, the solvent system comprises toluene and N-methyl-2-pyrrolidone.

In some step 2 aspects, the base is an inorganic base or an organic base. In some aspects, the organic base can be selected from among triethylamine, N,N-diisopropylethylamine, pyridine, 3-methylpyridine, dimethylaniline, N-methylimidazole, N-methylmorpholine, DABCO and DBU. In some step 2 aspects, the organic base is triethylamine. In some step 2 aspects, the organic base is N,N-diisopropylethylamine. In some aspects, the inorganic base can be selected from among sodium carbonate, potassium carbonate, sodium hydroxide, and potassium hydroxide.

Some aspects of the disclosure are directed to an alternative process for the preparation of compound 092 (N-(1-methylcyclopropyl)-2-(3-pyridyl) indazole-4-carboxamide). The process comprises forming a reaction mixture comprising compound 093a or 093b (4-chloro-2-(3-pyridyl) indazole or 4-bromo-2-(3-pyridyl) indazole), compound 070 (1-methylcyclopropanamine), a catalyst, a ligand, CO, a base, and a solvent system, and reacting the reaction mixture to form a reaction product mixture comprising compound 092 according to the following reaction scheme

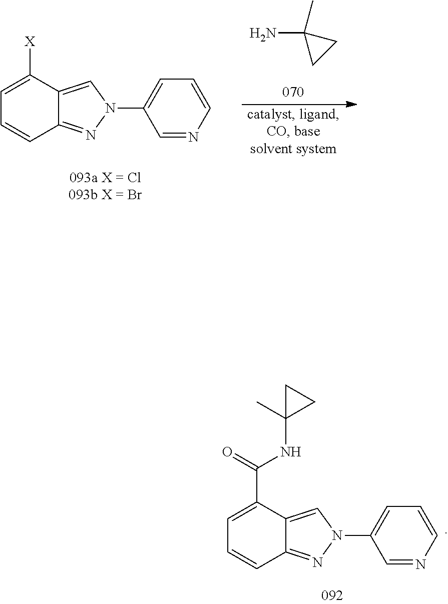

Aminocarbonylation reactions remain challenging even after several decades of work into the field. In particular, achieving good yield and selectivity using unreactive electrophiles such as aryl chlorides often requires the use of more forcing conditions such as high palladium loading (often at least 2 mol %) which can render a process uneconomical. Further, branched primary amines such as tert-butylamine are also often problematic due to their demanding sterics leading to poor yields in some cases as low as 46%. Strategies that utilize the formation of reactive activated ester-type species that are then converted to the desired amide via acyl transfer have been reported, but effective direct aminocarbonylation chemistry remains highly desirable. (Org. Chem. Front., 2022, 9, 2491; Org. Process Res. Dev. 2008, 12, 4, 566; Angew. Chem. Int. Ed. 2007, 46, 8460; ACS Catal. 2018, 8, 6, 5350)

In some aspects, the catalyst is a transition metal catalyst. In some aspects, the catalyst is selected from palladium catalysts, nickel catalysts, or platinum catalysts. In some such aspects the palladium catalyst is selected from palladium on carbon or palladium (II) acetate.

In some aspects, the ligand is a diphosphine ligand. In some such aspects the ligand is selected from 1,3-bis(dicyclohexylphosphino) propane and 1,3-bis(dicyclohexylphosphonium) propane bis(tetrafluoroborate).

In some aspects, the base is at least one weak inorganic base. In some such aspects, the base is a carbonate or a phosphate. In some such aspects, the base is selected from sodium carbonate, sodium bicarbonate, potassium carbonate, cesium carbonate, potassium bicarbonate, sodium phosphate dibasic, sodium phosphate tribasic, potassium phosphate dibasic, and potassium phosphate tribasic or combinations thereof. In some aspects, the base is aluminum hydroxide. In some aspects, the base is an acetate base. In some such aspects, the base is potassium acetate. In some aspects, the base is an alkoxide base. In some such aspects, the base is lithium t-butoxide, sodium t-butoxide, potassium t-butoxide or combinations thereof. In some aspects, the base is an organic base. In some such aspects, the base is a amine base. In some such aspects, the base is selected from among trimethylamine, triethylamine, tributylamine, DBU, DABCO, and N,N-diisopropylethylamine.

In some aspects, the solvent system comprises or predominantly comprises a polar aprotic solvent. In some aspects, the solvent system is selected from diglyme, dioxane, acetonitrile, DMF, DMAc, sulfolane, DMSO or some combination thereof. In some such aspects, the solvent system comprises or predominantly comprises dimethylsulfoxide.

In some aspects, the reaction atmosphere comprises or predominately comprises a mixture of CO and N₂. In some aspects, the reaction atmosphere comprises or predominately comprises CO. In some aspects, the reaction temperature is at least 100° C.

Some aspects of the disclosure are directed to an alternative process for the preparation of compound 092 (N-(1-methylcyclopropyl)-2-(3-pyridyl) indazole-4-carboxamide). the process comprising forming a reaction mixture comprising compound 061 (2-(3-pyridyl) indazole-4-carboxylic acid), compound 070 (1-methylcyclopropanamine), an activator, a base, and a solvent system, and reacting the reaction mixture to form a reaction product mixture comprising compound 092 according to the following reaction scheme

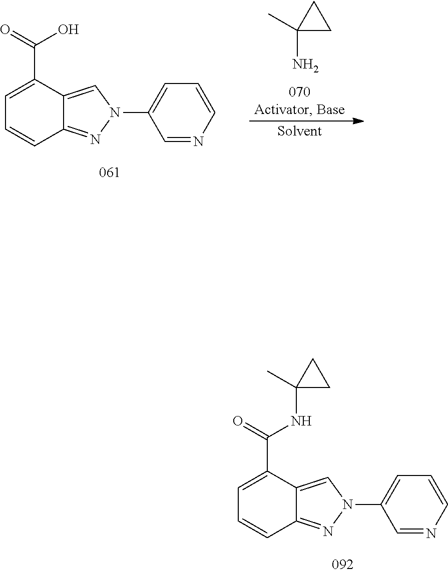

In some aspects, the activator is a acyl chloride, an anhydride, an alkyl chloroformate, a sulfonyl chloride, an acyl imidazole, or a triazine chloride. In some such aspects, the acyl chloride is selected from acetyl chloride, pivalic chloride, benzoyl chloride, or phosgene. In some such aspects, the anhydride is selected from acetic anhydride, pivalic anhydride, or di-tert-butyl dicarbonate. In some such aspects, the alkyl chloroformate is selected from methyl chloroformate, ethyl chloroformate, or isobutyryl chloride. In some such aspect, the sulfonyl chloride is selected from benzene sulfonyl chloride, p-toluene sulfonyl chloride, or methane sulfonyl chloride. In some such aspects, the acyl imidazole is selected from 1,1′-carbonyldiimidazole. In some such aspects, the triazine chloride is selected from cyanuric chloride or 2-chloro-4,6-dimethocyl-1,3,5-triazine.

In some aspects, the base is an organic base. In some such aspects, the base is a tertiary amine base such as triethylamine, diisopropylethylamine, N-methylmorpholine, N-methylpiperidine, diazobicylco[5.4.0]undec-7-ene, tributylamine, or N,N-dimethylbenzylamine. In some aspects, the base is a heterocyclic amine base such as pyridine, 2,6-lutidine, 3-picoline, imidazole, or N-methylimidazole.

In some aspects, the solvent system comprises or predominantly comprises at least one nonpolar solvent or polar aprotic solvent. In some such aspects, the solvent system comprises or predominantly comprises acetonitrile, N-methylpyrrolidine, toluene or combinations thereof. In some aspects, the solvent system comprises of predominantly comprises acetonitrile.

Some aspects of the disclosure are directed to an alternative process for the preparation of compound 092 (N-(1-methylcyclopropyl)-2-(3-pyridyl) indazole-4-carboxamide). the process comprising forming a reaction mixture comprising compound 038 (methyl 2-(3-pyridyl) indazole-4-carboxylate), compound 070 (1-methylcyclopropanamine), a base, and a solvent system, and reacting the reaction mixture to form a reaction product mixture comprising compound 092 according to the following reaction scheme

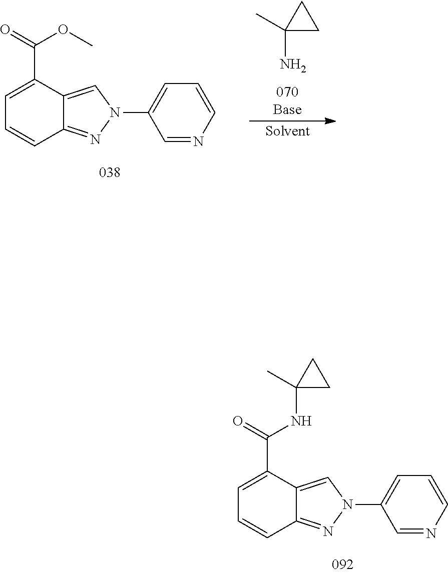

In some aspects, the base is an organometallic base. In some aspects the base is selected from organomagnesium bases or organoaluminum bases. In some such aspects, the base is an organomagnesium base such as, but not limited to, isopropyl magnesium chloride, isopropyl magnesium bromide, ethyl magnesium chloride, ethyl magnesium bromide, methyl magnesium chloride, or methyl magnesium bromide. In some aspects, the base is an organoaluminum base. In some such aspects, the organoaluminum base is a trialkylaluminum base. In some such aspects the base is selected from trimethylaluminum, triethylaluminum, or triisobutylaluminum. In some aspects, the base is lithium aluminum hydride.

In some aspects, the solvent system comprises or predominantly comprises a nonpolar solvent or polar aprotic solvent. In some such aspects, the solvent system comprises or predominantly comprises tetrahydrofuran, 2-methyl tetrahydrofuran, 1,4-dioxane, diethylene glycol dimethyl ether, 1,2-dimethyoxyethane, diethyl ether, diisopropyl ether, methyl-tert-butyl ether, or cyclopentylmethyl ether.

Some aspects of the disclosure are directed to a process for preparing a compound 092 salt, the process comprising forming a reaction mixture comprising compound 092, a solvent system, an acid, and reacting the reaction mixture to form a reaction product mixture comprising compound 092 salt according to the following reaction scheme

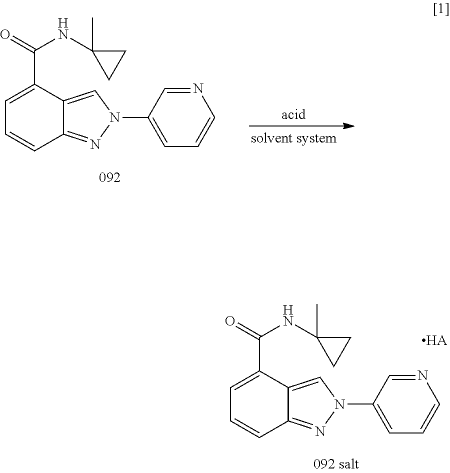

In some aspects, the solvent system comprises or predominantly comprises a nonpolar solvent, a polar solvent, or a combination thereof.

In some aspects, the acid is an inorganic acid or an organic acid. In some aspects, the inorganic acid can be selected from among hydrochloric acid, hydrobromic acid, sulfuric acid, phosphoric acid and nitric acid. In some aspects, the organic acid can be selected from among acetic acid, glucuronic acid, oxalic acid, malic acid, citric acid, tartaric acid, maleic acid, fumaric acid, succinic acid, benzoic acid, methanesulfonic acid, benzenesulfonic acid, para-toluenesulfonic acid and trifluoroacetic acid.

In some aspects, compound 092 may be isolated. In some aspects, compound 092 may be crystallized from a solution thereof in an organic solvent by adding compound 092 seed crystals to the solution of compound 092 followed by charging of water over a period of time and cooling. Crystalline compound 092 may then be isolated by methods known in the art (such as filtration or centrifugation) and optionally washed with water. The isolated crystalline compound 092 may then be optionally dried. In such aspects, step 2 may further comprise the following order of steps: (i) exchanging the solvent system to an organic solvent system suitable for crystallization and form a solution of compound 092 in the organic solvent system; (ii) adding water and optional compound 092 seed crystals thereto to form a slurry; (iii) cooling the slurry; and (iv) isolating crystalline compound 092.

In some aspects, the organic solvent is a polar solvent. In some aspects, the polar solvent is selected from water, a ketone, a nitrile, an amide, and a C₁₋₄ alcohol, and combinations thereof. In some aspects, the polar solvent is selected from water, N-methylpyrrolidone, acetonitrile, dichloromethane, dimethylformamide, dimethylacetamide, dimethylsulfoxide, sulfolane, methylethylketone, ethanol, methanol, propanol, butanol, and isopropanol, and combinations thereof.

In some aspects, the solution of compound 092 therein is a concentrated solution but below the saturation point at an elevated temperature of the solvent. Non-limiting examples of some suitable temperatures include, from about 75° C. to about 95° C. or from about 85° C. to about 95° C. However, one skilled in the art will recognize that higher or lower temperature ranges may be suitable depending on the boiling point of the solvent. In some aspects, from about 1% to about 5% of compound 092 seed crystals are added followed by the addition of water over a period of time to form a slurry of crystalline compound 092. In some aspects, the water addition step may be done at approximately the same temperature as the solution of compound 092 in the organic solvent. The volume ratio of water to organic solvent is suitably about 0.5:1, about 0.75:1, about 1:1, about 1.25:1, about 1.5:1, about 2:1, about 2:5:1 or about 3:1, and any range constructed therefrom, such as from about 0.5:1 to about 3:1 or from about 1:1 to about 1.5:1. The addition of water may suitably done over from about 1 hour to about 10 hours, such as about 1 hour, about 2 hours, about 3 hours, about 4 hours, or about 5 hours. The slurry of compound 092 may then be cooled over a period of time to complete compound 092 crystallization. The cooling time is suitably from about 1 hour to about 24 hours, or from about 2 hours to about 12 hours, such as about 3 hours, about 5 hours or about 8 hours. The final temperature is suitably about 5° C., about 10° C., about 15° C., about 20° C., or about 25° C. Crystalline compound 092 may then be isolated, washed with water, and dried. In some such aspects, the polar solvent is a C₁₋₄ alcohol, or is ethanol, and crystalline compound 092 is predominantly of Form A. In some aspects, the polar solvent is ACN, and crystalline compound 092 is predominantly of Form B.

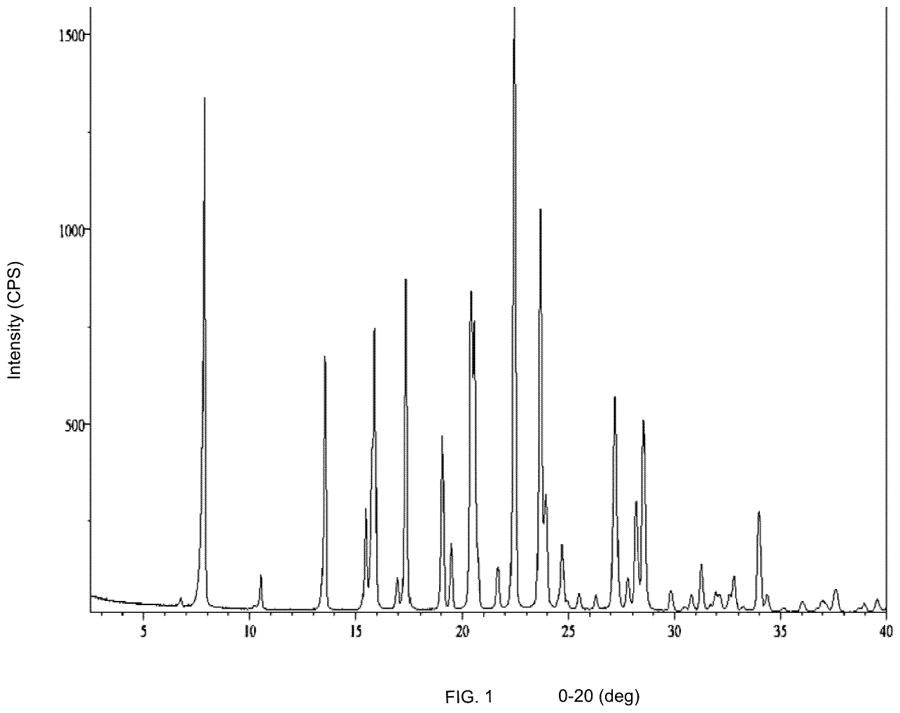

In some aspects, crystalline compound 092 Form A is characterized by a X-ray powder diffraction pattern generally in accordance with FIG. 1.

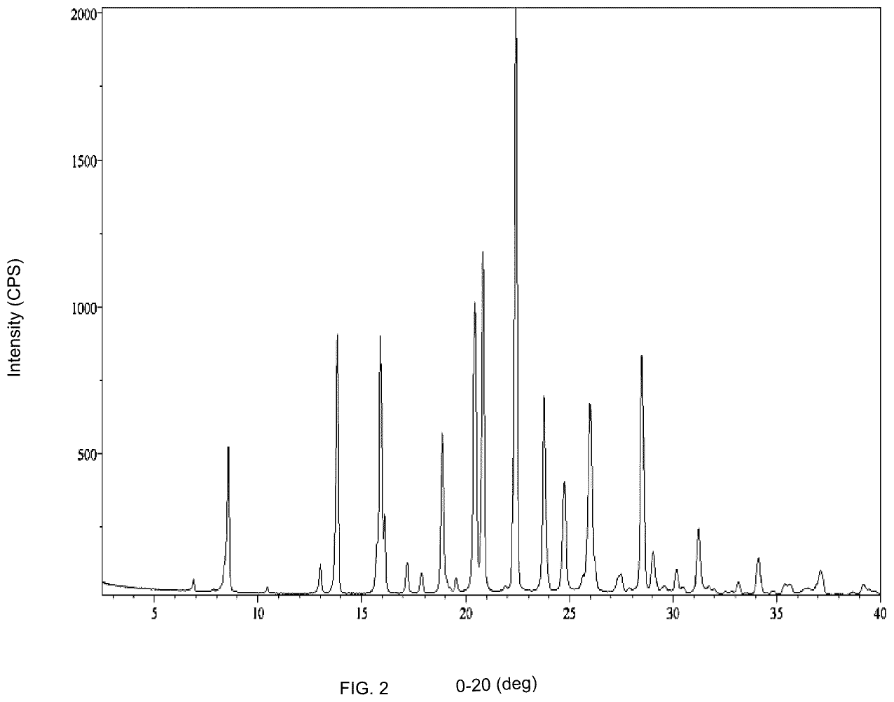

In some aspects, crystalline compound 092 Form B is characterized by a X-ray powder diffraction pattern generally in accordance with FIG. 2.

### EXAMPLES

### Example 1

Compound 223 was methylated with chloromethane to produce compound 775 as follows:

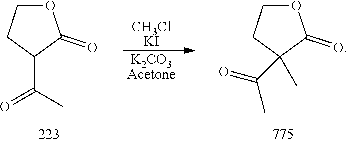

A 25 L autoclave was charged with K₂CO₃ powder (325 mesh, 1.55 kg, 1.2 eq.), potassium iodide (155.4 g, 0.1 eq) and acetone (7.2 L, 6V) at 25-30° C., followed by addition of α-acetylbutyrolactone (compound 223) (1.2 kg, 1 eq.). The autoclave vessel was closed and methyl chloride (2 eq) was charged into the autoclave until the pressure reached ˜25 psi. The reaction mixture was heated and the temperature maintained at 40-45° C. until <2 A % compound 223 remained as monitored by HPLC. After completion of the reaction, the reaction product mixture was cooled to room temperature, filtered and washed with acetone. The filtrate containing crude compound 775 was concentrated under vacuum at 40-45° C. to yield a brown liquid (1.3 kg). HPLC of isolated crude material: compound 223 (non-detect), compound 775 (94.13 A %), O-methylated byproduct (1.01 A %). The yield of compound 775 was 91.2% (93.2 wt %).

### Example 2

Methylation of compound 223 with chloromethane without addition of KI: Into an autoclave was charged K₂CO₃ (1.4 eq) and acetone (6V), then α-acetylbutyrolactone (compound 223, 2 g) was added. Methyl chloride (condensed at −30° C., 3 eq) was charged into the autoclave and the autoclave closed. The reaction mass was heated to 55-60° C. and maintained for 18 h. The progress of the reaction was monitored by HPLC. After completion of the reaction, the reaction mass was cooled to room temperature, filtered and washed with acetone. The filtrate containing compound 775 was concentrated under vacuum at 40-45° C. MTBE was charged into the crude product and stirred, then the mass was filtered and washed with MTBE. The filtrate was concentrated under vacuum at 40° C. to obtain crude compound 775 as a pale brown liquid. HPLC of isolated crude material: compound 223 (28.32 A %), compound 775 (38.31 A %), O-methylated byproduct (23.9 A %).

### Example 3

Methylation of compound 223 with dimethyl sulfate to produce compound 775: Compound 223 (1 eq.), solvent, dimethyl sulfate (1.2 eq.), base (1.4 eq.), and PTC were charged to a multi-neck round-bottom flask, and stirred for 1 h at 25-30° (reactions 6 and 7) or 50-55° (reactions 1-5 and 8-12). The reaction was monitored for completion by HPLC. The results are reported in Table 1, where “ND” refers to not detected, and “NR” refers to not reported.

TABLE 1

| | | | | | | | O- |
|---|---|---|---|---|---|---|---|
| | | | | | | | methylated |
| | | PTC | Solvent | Time | 223 | 775 | byproduct |
| Reaction | Base | (eq.) | (V) | (h) | (A %) | (A %) | (A %) |
| 1 | NaOH | TBAI | Toluene:Water | 18 | 2.04 | 12.89 | 65.06 |
| | | (0.02) | (5:1, 6 V) | | | | |
| 2 | NaOH | TBAI | Toluene:Water | 18 | ND | 10.77 | 62.13 |
| | | (0.02) | (3:3, 6 V) | | | | |
| 3 | NaOH | TBAI | THF | 2 | 0.41 | 8.35 | 68.76 |
| | | (0.02) | (5 V) | | | | |
| 4 | K₂CO₃ | TBAB | ACN | 2 | ND | 10.4 | 83.61 |
| | | (0.02) | (5 V) | | | | |
| 5 | | TBAB | ACN | 16 | 2.32 | 12.65 | 48.99 |
| | | (0.02) | (5 V) | | | | |
| 6 | K₂CO₃ | None | Toluene:Water | 2 | ND | ND | 73.69 |
| | | | (3:3, 6 V) | | | | |
| 7 | NaOH | TBAB | Toluene:Water | 3 | 4.55 | 9.42 | 57.88 |
| | | (0.02) | (3:3, 6 V) | | | | |
| 8 | NaOH | TBAB | Toluene:Water | 1 | 4.2 | 8.02 | 59.89 |
| | | (0.1) | (3:3, 6 V) | | | | |
| 9 | NaOH | None | THF | 5 | ND | 9.81 | 41.59 |
| | | | (5 V) | | | | |
| 10 | K₂CO₃ | None | Acetone | 5 | 0.55 | 7.35 | 84.41 |
| | | | (6 V) | | | | |
| 11 | NaOH | TBAB | THF:water | 5 | 5.12 | 13.8 | 63.8 |
| | | (0.02) | (3:3, 6 V) | | | | |
| 12 | NaOH | TBAB | THF | NR | 13.15 | 8.66 | 71.2 |
| | | (0.02) | (5 V) | | | | |

### Example 4

Preparation of Compound 069: Into a round-bottom flask was charged concentrated HCl (37%, 3V) and heated to 55-60° C. Compound 775 (1.3 kg, 92.1 wt %) was added slowly into the hot HCl with evolution of CO₂ gas. The reaction was maintained at this temperature for 1 hour, then cooled to room temperature. DCM was charged into the reaction mass and stirred. The two layers were separated, and the DCM removed under vacuum at 40° C. to afford crude 069 (1.14 kg, 91.2% yield, 92.2 wt % by GC).

### Example 5

Preparation of Compound 079: Into a round-bottom flask was charged KOH powder (1.05 eq.) and water (2V) at room temperature and the solution was heated to 50-55° C. Compound 069 (1.14 kg, 92.2 wt %) was charged to the heated reaction mass over 1 hour and held at this same temperature until compound 069 was <2 A % by GC. Upon completion of the reaction, the reaction mass was cooled to 15-20° C. and the pH was adjusted to ˜pH=2 with 25% aqueous H₂SO₄. The two layers were separated and the crude oil (751 g, 87.2% yield, 89 wt % assay by GC) was distilled under vacuum (10-20 mbar) at 45-90° C. to isolate compound 079 (591 g, 60.4% yield over 3 steps, 94 wt % by GC).

### Example 6

Preparation of Compound 200: Into a multi-neck round-bottom flask was charged 9.6% NaOCl (3.5 eq) and the solution was cooled to 10-15° C. Compound 079 (500 g) was charged slowly into the cooled solution. The reaction was held at room temperature until compound 079 was <2 A % by HPLC. The reaction mass was cooled down to 10-15° C. and quenched with aqueous sodium bisulfite (˜0.5 eq). The CHCl₃ and aqueous layers were separated, and the aqueous layer washed once with MTBE (5V). The aqueous layer was then cooled back to 10-15° C. and acidified to pH=2 with concentrated H₂SO₄ (˜630 mL) while maintaining the temperature <25° C. The aqueous layer was extracted with DCM (2×5V) and the combined DCM layers reduced to 5V by distillation. The solution of compound 200 in DCM was carried forward into the next step.

Preparation of Compound 144: Into a multi-neck round-bottom flask was charged a solution of compound 200 in 5V DCM and DMF (0.03 eq). Thionyl chloride (1.05 eq) was added slowly. After addition was complete, the reaction was held at room temperature until compound 200 was <2.0 A % by HPLC. In a separate multi-neck round-bottom flask, aqueous NH₃ (30% in water, 2.5 eq) was charged and cooled to 0-5° C. The acid chloride solution was charged slowly into the cooled ammonia solution and the reaction was held for 1 hour. Water (2V) was charged into the reaction mass and the DCM was distilled off at 40° C. The reaction mass was cooled to 10-15° C., filtered, and the filter cake washed with water (IV). The solid was dried at 40-45° C. to yield compound 144 (420 g, 82% yield over 3 steps, 98.6 wt % by HPLC).

### Example 7

Preparation of 1-methylcyclopropanamine (compound 070)

Preparation of Compound 070: 50% aqueous NaOH (4.0 eq) and water (5V) were charged into a round-bottom flask and cooled to 0-5° C. Compound 144 (400 g) was charged and to this slurry was slowly added NaOCl (10.1%, 1.5 eq.), keeping the temperature <10° C. The reaction mass was held at 0-5° C. for 2 hours. The reaction mass was then allowed to come to 20-25° C., held for 2 hours, heated to 40-45° C. and held for 2 hours. Compound 070 was distilled from the reaction mass and collected along with water (290 g, 79.5 wt % by GC, 80.4% yield). Compound 070 was further subjected to fractional distillation with a 1-foot packed column to yield compound 070 with a purity of 95.6 wt % by GC (70.6% yield).

### Example 8

Preparation of 2,2-dichloro-1-methyl-cyclopropanecarboxylic acid: Into a 500 mL, 4-neck round bottom flask, KOH (56.1 g, 1 mol) was added in a portion-wise manner into a mixture of chloroform (81 mL, 1 mol), methyl methacrylate (26.7 mL, 0.25 mol) and benzyltriethylammonium chloride (2.85 g, 0.012 mol) at 15-20° C. The reaction was maintained at 15-20° C. until complete consumption of starting material was confirmed.

Upon completion of the reaction, methanol (22 mL, 0.52 mol) and KOH (21.04 g, 0.38 mol) were added and the mixture was heated to 65° C. for 6-7 hr. Water (300 mL) was then added and the reaction mass was acidified with concentrated HCl to pH=1-2. The aqueous layer was extracted with toluene (250 mL), the layers separated, the organic layer dried over sodium sulfate, and the solvent removed under vacuum to give 2,2-dichloro-1-methyl-cyclopropanecarboxylic acid as a brown crystalline solid (41 g) in 88% yield (85 wt % by GC).

### Example 9

Example 8 was repeated with ethyl methacrylate. The yield of 2,2-dichloro-1-methylcyclopropyl carboxylic acid was 90%.

### Example 10

In a dry autoclave reactor, a mixture of t-butanol (100 mL, 1.04 mol), 2,2-dichloro-1-methyl-cyclopropanecarboxylic acid (5 g, 0.029 mol), KOH (11.76 g, 0.17 mol) and 5% Pd/C (1.04 g, 0.0002 mol) was stirred at 130° C. under hydrogen (45 bar). The reaction was held for 12-14 hrs. The reaction mass was filtered on a celite bed and washed with t-butanol. The filtrate was distilled to remove most of the t-butanol. The filtrate was then charged with water (50 mL) and was acidified with concentrated HCl to pH=1-2, followed by extraction with dichloromethane to yield a thick brown liquid comprising 1-methylcyclopropylcarboxylic acid (2.1 g) in 42.5% yield (59.3 wt % by HPLC).

### Example 11

Example 10 was repeated but with 2,2-dibromo-1-methyl-cyclopropanecarboxylic acid. A yield of 48% 1-methylcyclopropylcarboxylic acid was achieved.

### Example 12

Under nitrogen atmosphere, THF (120 mL, 1.44 mol) was charged into a 3 L 4-neck round bottom flask, followed by 2,2-dichloro-1-methyl-cyclopropanecarboxylic acid (20 g, 0.12 mol). Aqueous 15% methanol (790 mL) and sodium (109.88 g, 4.72 mol) was added simultaneously in portions over a period of ˜4-5 hours, then held for an additional 6-7 hours. The majority of the organic solvent was removed, the remaining residue diluted with water and acidified with concentrated HCl to pH=1-2. The reaction mass was extracted with dichloromethane and the solvent removed under vacuum to provide 1-methylcyclopropylcarboxylic acid as a brown liquid (11.4 g, 93% yield, 92.9 A % GC).

### Example 13

2,2-dichloro-1-methyl-cyclopropanecarboxylic acid (25 g, 0.14 mol), zinc dust (384.6 g, 5.91 mol) and KOH pellets (33.2 g, 0.59 mol) were refluxed together in 300 mL t-butanol for 46-48 hr. The reaction mass was cooled to 30° C., filtered under vacuum and the cake was washed with t-butanol. The combined filtrates were distilled under vacuum until the majority of the solvent was removed. Water was then charged into the residue, followed by acidification with concentrated HCl to pH=1-2. The aqueous layer was extracted with dichloromethane and the solvent was evaporated to give 1-methylcyclopropylcarboxylic acid as a pale, yellow liquid (13.9 g, 92.8% yield, 98.7 wt % by HPLC).

### Example 14

In a 2 L 4-neck round-bottom flask, 1-methylcyclopropanecarboxylic acid (48 g, 0.48 mol) and dimethylformamide (1.12 mL, 0.01 mol) were charged into dichloromethane (460 mL, 8.48 mol) under nitrogen atmosphere. The reaction mass was cooled to 0-5° C. and thionyl chloride (40 mL, 0.55 mol) was added slowly, maintaining the reaction mass temperature <5° C. After addition was complete, the temperature was raised to 23-25° C. and was held until consumption of starting material was confirmed. In a separate 2 L 4 neck round-bottom flask was charged methanolic ammonia (268 mL, 1.44 mol) and cooled to 0-5° C. The acid chloride solution was slowly added to the methanolic ammonia, keeping the temperature between 5-10° C. After addition was complete, the temperature was brought to 20-25° C. and held for 1 hr. Upon reaction completion, most of the solvent was distilled off. Dichloromethane (800 mL, 7.0 mol) was added to the residue, the solution was filtered, and the solvent was evaporated under vacuum to give 1-methyl cyclopropanecarboxamide (44.3 g) as white solid (88% yield, 95 wt % by HPLC).

### Example 15

Preparation of 1-(1-methylcyclopropyl) ethanone oxime: A reaction flask was charged with compound 079 (20 g, 94.3 wt %), EtOH (100 mL, 5V), and NH₂OH·HCl (19.7 g, 1.45 eq.) at 22° C. with stirring. NaOAc (24.6 g, 1.55 eq.) was charged in one portion. The reaction mass was stirred at room temperature and monitored by HPLC. When conversion was complete, the reaction mass was quenched slowly with saturated aqueous NaHCO₃ (400 mL, 20V). The reaction mass was extracted with EtOAc (3×10V). The combined organic layers were dried over NaSO₄ and concentrated under reduced pressure. 1-(1-methylcyclopropyl) ethanone oxime was obtained as a white solid (21.1 g, 95.3 wt %, 92% yield).

### Example 16

Preparation of N-(1-methylcyclopropyl) acetamide: 1-(1-methylcyclopropyl) ethanone oxime (20.0 g), TsCl, (1.6 g, 0.05 eq.) and MeCN (120 mL, 6V) were charged to a reaction flask and stirred. The reaction mass was heated to 80° C. and reaction progress was monitored by HPLC. Upon complete conversion, the reaction mass was cooled to ambient temperature and activated carbon was charged (1.5 g, 7.5% wt/wt) and stirred for 3 hours. The slurry was filtered through a bed of celite and washed with MeCN (100 mL, 5V). The filtrate was concentrated under reduced pressure to obtain crude compound N-(1-methylcyclopropyl) acetamide as an orange-colored solid (21.1 g, 80.2 wt %, 89% yield).

### Example 17

Preparation of 1-methylcyclopropanamine (070): Compound N-(1-methylcyclopropyl) acetamide (6 g) and water (42 mL, 7V) were charged into a reaction flask with stirring. The reaction was heated to 80° C. Concentrated sulfuric acid (5.38 mL) was charged into the heated solution in 215 μL portions at 15 min intervals over 6 hours. The reaction was held at 80° C. and monitored by GC. Upon completion of conversion (˜42 hours), the reaction mass was cooled to 0-5° C. The pH was adjusted to pH=10-11 with 50% aq. NaOH (˜15 g) while maintaining the temperature <15° C. The reaction mass was analyzed for 1-methylcyclopropanamine (1.86 g, 60% yield).

### Example 18

Preparation of 1-methylcyclopropanamine (070): Charged acetonitrile (2 kg), tetrahydrofuran (14.1 kg), and titanium (IV) isopropoxide (16.2 kg) into a jacketed reactor at 25-30° C. and agitated. Cooled the reaction mass to 5-10° C. and charged ethylmagnesium chloride (49 kg, 2 M in tetrahydrofuran), maintaining the internal temperature <20° C. After addition was complete, the reaction was allowed to warm to 25-30° C. and stirred for 2 hours. Conversion of acetonitrile was monitored by GC. Once the reaction was complete, the reaction mass was cooled to 5-10° C. and charged boron trifluoride diethyl etherate (14.1 kg), maintaining the internal temperature <20° C. Once addition was complete, the reaction mass was allowed to warm to 25-30° C. and stirred for 1 hour. The reaction was then cooled to 5-10° C. and a solution of potassium sodium tartrate tetrahydrate (8.4 kg dissolved in 32 kg water) was charged into the reaction mass, maintaining the internal temperature <20° C. Once addition was complete, the reaction mass was allowed to warm to 25-30° C., stirred for 1 hour, then filtered to remove the solids. The filtrate was collected and stored separately. The filter cake was re-charged back into the reactor and re-slurried with a tetrahydrofuran/water solution (10 kg/12 kg). After stirring for 30 minutes, the slurry was filtered. The filtrate was collected and stored separately, and the filter cake was subjected to re-slurry once more. After filtration, the filter cake was washed with a tetrahydrofuran/water solution twice (4.4 kg/5 kg). The combined filtrates were charged into a jacketed reactor and the organic solvent was distilled off. After completion of distillation, the reaction mass was cooled to 5-10° C. and the pH of the reaction mass was adjusted with aqueous sodium hydroxide (5.6 kg in 18 kg water) to PH ˜12. After stirring the reaction mass for a period of time, compound 070 was isolated by distillation, along with water (7 kg, 30.1 wt %, 60.9% yield).

### Example 19

Preparation of 1-methylcyclopropanamine (070): Charged acetonitrile (15 kg), tetrahydrofuran (105.6 kg), and titanium (IV) isopropoxide (119.4 kg) into a jacketed reactor at 25-30° C. and agitated. Cooled the reaction mass to 5-10° C. and charged ethylmagnesium chloride (383.6 kg, 2 M in tetrahydrofuran), maintaining the internal temperature <20° C. After addition was complete, the reaction was allowed to warm to 25-30° C. and stirred for 2 hours. Conversion of acetonitrile was monitored by GC. Once the reaction was complete, the reaction mass was cooled to 5-10° C. and charged boron trifluoride diethyl etherate (103.7 kg), maintaining the internal temperature <20° C. Once addition was complete, the reaction mass was allowed to warm to 25-30° C. and stirred for 1 hour. The reaction was then cooled to 5-10° C. and a solution of potassium sodium tartrate tetrahydrate (63 kg dissolved in 240 kg water) was charged into the reaction mass, maintaining the internal temperature <20° C. Once addition was complete, the reaction mass was allowed to warm to 25-30° C., stirred for 1 hour, then the organic solvent removed by distillation. Partway through the distillation, 300 kg water was charged into the reaction mass. After completion of distillation, the reaction mass was cooled to 5-10° C. and the pH of the reaction mass was adjusted with 50% aqueous sodium hydroxide (138.6 kg) to pH ˜12. After stirring the reaction mass for a period of time, compound 070 was isolated by distillation, along with water (fraction 1-40.6 kg, 34.6 wt %; fraction 2-88.6 kg, 1.3 wt %; 58.7% yield).

### Example 20A

Preparation of 2-chloro-6-nitro benzaldehyde (compound 378)

To a reactor was charged a solution of 2-(bromomethyl)-1-chloro-3-nitrobenzene (compound 181a) (92.6 A % by HPLC) in dichloromethane and the solution was concentrated to 1 V. DMF (1.0 V) was charged to the reactor and the solution was concentrated to 1 V. DMF (2.0 V) was charged to the reactor. Trimethylamine-N-oxide (2.2 eq., 50 wt % in water) was charged to the reactor and the mixture was heated to 50° C. The reaction mass was held at 50° C. until HPLC indicated completion of the reaction to compound 378. The reaction mass was 83.7 A % compound 378 by HPLC analysis.

### Example 20B

Preparation of 4-chloro-2-(3-pyridyl)indazole (compound 093a)

2-chloro-6-nitrotoluene (compound 339) (200.0 g) was added to a reactor under a flow of nitrogen and dissolved in 5 V dichloromethane (DCM) with agitation. Water (1 V), N-bromosuccinimide (1.25 equiv), and azobisisobutyronitrile (0.05 equiv) were added, and 400 W Hg lamps were turned on to initiate the photochemical bromination at a reaction temperature of 40-45° C. The reaction was sampled periodically for IPC analysis by HPLC until compound 339 was <1% by area. The reaction was cooled to room temperature and quenched with 4 V of a 10% solution of sodium sulfite in water. The aqueous layer was separated, and the DCM layer was washed with an additional 4 V of water. The aqueous layer was separated, combined with the first aqueous layer, and extracted with 2 V DCM. All of the organic layers were combined and washed with 4 V of a 5% sodium sulfite solution. The organic layer was separated again and washed with 4 V of a saturated sodium chloride solution. The organic layer was separated and distilled to 1 V. Acetonitrile (2 V) was added, and the combined solvents were distilled to 1 V ACN at room temperature. This solution was taken on to the next step.

The solution of 2-(bromomethyl)-1-chloro-3-nitrobenzene (compound 181a) from Step 1 (˜270 g of compound 181a) was charged to the reactor, and acetonitrile (970 mL) was added. Sodium carbonate (181 g) and water (1269 mL) were charged to the reactor under nitrogen, and the reaction mass was stirred at room temperature for 10 minutes. The reaction was then heated to 80-85° C. for 14-16 hours or until compound 181a was no more than 1% by area according to analysis by HPLC. The reaction mixture was cooled to 50° C., and the acetonitrile was distilled until its content was <5% by area according to analysis by gas chromatography. The reaction mass was cooled to 25-30° C., and 500 mL toluene were added. The mixture was stirred for 20 min at this temperature before the layers were allowed to separate. The toluene layer was kept aside, and the aqueous layer was charged back into the reactor, where it was extracted with another 250 mL of toluene. Both toluene layers were combined and washed with 212 mL water. The toluene solution containing (2-chloro-6-nitrophenyl) methanol (compound 050) was taken onto the next step.

The toluene solution of compound 050 was charged to the reactor at 25-30° C., and the reactor was inerted with nitrogen. Tetrabutylammonium bromide (TBAB, 1.04 g) and sodium bicarbonate (108.7 g) were added under nitrogen, followed by slow addition over 2 h of NaOCl solution (1121.6 g), keeping the reaction temperature below 30° C. The pH of the solution was between 9 and 10; if above 10, the pH was adjusted with 6N HCl until it was in the desired range. The reaction mass was stirred at 25-30° C. for 2 h or until compound 050 was no more than 1% by area according to HPLC analysis. Stirring was discontinued, and the layers were allowed to separate. A 5% aqueous solution of sodium thiosulfate was prepared by dissolving 10.55 g Na₂S₂O₃ in 212 mL water. This solution was added to the reaction mass, which was stirred for an additional 25-30 min at room temperature, at which time the layers were separated. The toluene layer was washed with water (2×212 mL), and the toluene solution containing 2-chloro-6-nitro benzaldehyde (compound 378) was taken onto the next step.

Under nitrogen, the toluene layer containing compound 378 was charged to the reactor along with 3-aminopyridine (98.8 g) and p-toluenesulfonic acid (0.095 g). The reaction mass was heated to 105-110° C.; the slurry became a dark homogeneous solution as the temperature was increased. The reaction was stirred at this temperature for 12-16 h with azeotropic distillation of water until compound 378 was no more than 2% by area according to HPLC analysis. The reaction mixture was cooled to room temperature, and the toluene solution of 1-(2-chloro-6-nitrophenyl)-N-(pyridine-3-yl) methanimine (compound 003a) was taken onto the next step.

Triethylphosphite (670.9 g) was added to the toluene solution of compound 003a at 25-30° C. under nitrogen. The reaction mixture was heated to 105-110° C. for 20-25 h or until compound 003a was no more than 2% by area according to HPLC analysis. At this time, the temperature was reduced to 45-50° C., and toluene was distilled under 50 mbar vacuum until it was <5% by area percent according to GC analysis. The reaction mass was cooled to 25-30° C., and 92.7 mL of isopropanol was added. The slurry was stirred for 30 min prior to addition of 1110 mL of water. The reaction mass was then cooled to 10-15° C. and stirred for 2 h. The resulting solid was isolated via filtration and washed with 277.5 mL water. The solid was then slurried with 92.7 mL isopropanol and 370 mL of water, filtered, and washed with 370 mL water. The solid was dried under vacuum at 50° C. until the water content was less than 1% according to analysis by KFT. The assay corrected yield for steps 1-5 was 45-47%, corresponding to 98.0-100 weight percent of 4-chloro-2-(3-pyridyl) indazole (compound 093a).

Compound 093a (56 kg), DMSO (4 V), and water (1 V) were added to the reactor under nitrogen purge. Then Pd/C (50 wt % water, 0.0015 equiv), 1,3-bis(dicyclohexylphosphino)propane bis(tetrafluoroborate) (0.0030 equiv), and potassium carbonate (1.5 equiv) were added under nitrogen purge. The reaction mass was degassed with nitrogen, the pressure was released, and the reactor was pressurized with 130-135 psi carbon monoxide and heated to 100-110° C. for 26 h or until 093a (4-chloro-2-(3-pyridyl) indazole) was no more than 3% by HPLC area percent analysis. The reaction mass was cooled to room temperature and diluted with 5 V water followed by stirring for 25-30 minutes. Then 1V 10% NaOH aqueous solution was added with an additional 15 minutes stirring before filtering through a bed of Celite. The aqueous reaction mass was extracted with toluene (2×2.25 V, and the separated aqueous layer was acidified with 3 N HCl until a pH of 3-4 was obtained. The resulting slurry was stirred at room temperature for 1 hour, at which point crystals of compound 061 (2-(3-pyridyl) indazole-4-carboxylic acid) were isolated via filtration, washed with water, and dried to a constant weight (54.4 kg, 93.5%).

### Example 21

The above procedure was repeated using compound 093b (4-bromo-2-(3-pyridyl) indazole). Analysis by UHPLC indicated the following area percent for the reaction mixture: compound 061 (2-(3-pyridyl) indazole-4-carboxylic acid) (93.5%), compound 093b (0.3%).

### Example 22

Preparation of 4-chloro-2-(3-pyridyl)indazole (compound 093a)

In an appropriately-sized reactor equipped with column and condenser (or Dean-Stark trap), 2,6-dichlorobenzaldehyde (compound 150) (450 g) and p-toluenesulfonic acid (0.0005 equiv) were dissolved in 5 V toluene. The solution was heated to 105-110° C. with continuous distillation of water until reaction completion (˜16 h). The toluene was distilled, and the resulting crude solid was suspended in 0.5 V isopropanol and 2 V hexane. The slurry was stirred for 1 hour at room temperature, and the solid was isolated via filtration and drying in vacuo at 45° C. or below. The yield of 1-(2,6-dichlorophenyl)-N-(pyridin-3-yl) methanimine (compound 086) was 560 g (86.7%).

Compound 086 (100 g) was dissolved in 10 V methanol at room temperature in a 2 L round bottom flask. The solution was cooled to 0-5° C., and sodium borohydride (0.8 equiv) was added in four portions over an hour. The reaction completed in 2 hours, after which time the reaction was quenched with 20 V water, stirred, and allowed to come to room temperature over an hour. The resulting slurry was filtered, and the solid was washed with 3 V water and dried in vacuo at 50° C. to yield 88.0 g (87%) of N-(2,6-dichlorobenzyl)pyridin-3-amine (compound 084).

Compound 084 (300 g) was dissolved in 6 V DCM in areactor at room temperature. The solution was cooled to 0° C. with stirring, and then NaNO2 (1.2 equiv) and p-toluenesulfonic acid (1.2 equiv) were added in four portions each while maintaining the temperature at 0° C. The reaction mass was then allowed to warm to room temperature and stirred for 6 hours. The reaction was then filtered through a bed of Celite, and the DCM solution was distilled to dryness to yield a brown solid compound 085 (300 g, 90%).

In a glass reaction flask, compound 085 (35.0 g) was dissolved in 13.5 V methanol. The solution was stirred at room temperature as thiourea dioxide (3.5 equiv) was added. The reaction mass was stirred for 5 minutes before cooling to −10° C. Sodium hydroxide (3 equiv of IN solution in water) was added, and the reaction mass was warmed to 50° C. and stirred for 5 hours. The reaction was then cooled to 25-30° C. and diluted with 15 V ethyl acetate. The organic layer was separated and washed with brine. The organic layers were combined and dried over sodium sulfate before filtration and distillation of the ethyl acetate to yield 30 g of a crude sample of compound 3-(1-(2,6-dichlorobenzyl) hydrazinyl)pyridine (compound 048) free base.

Crude compound 048 free base (18.0 g) was dissolved in 4 V ethyl acetate. The solution was cooled to 15-20° C., and 5 equiv of HCl in isopropanol were added slowly while maintaining this temperature. A solid formed upon addition of HCl. The reaction mass was stirred for 1 hour, and the solid was isolated via filtration and washing with 2 V ethyl acetate. It was dried in vacuo at 50° C. to yield 14 g of 048 (68%). The sample was ˜95.7 area percent of compound 048 HCl salt contaminated with 3.0 A % of the amine byproduct according to UHPLC analysis.

Column and method info: Acquity BEH C18 (2.1 mm×50 mm, 1.7 um), Mobile phases A: 0.05% formic acid in water, B: 0.05% formic acid in acetonitrile. Time (min)/% B: 0/10; 0.4/10; 4/100; 6/100; 6.1/10; 7/10. Column temp 35° C. Flow rate 0.5 mL/min.

Compound 048 HCl salt (15.0 g) was suspended in 10 V dichloromethane, and the resulting slurry was cooled to 5-10° C. Triethylamine (5 equiv) was added, and the solution was stirred for 15 minutes before acetic anhydride (3 equiv) was added. The reaction was warmed to room temperature and stirred for 8 hours. The solvents were distilled to 2 V, and the mass was cooled to 25-30° C. and held at this temperature as 5 V water were added over 30 minutes. The resulting slurry was filtered, and the solid was washed with 3 V water prior to drying in vacuo at 50° C. 12.2 g of N′-(2,6-dichlorobenzyl)-N′-(pyridin-3-yl) acetohydrazide (compound 083) were isolated in 80.2% yield.

Compound 083 (5.0 g) was charged in a reactor and dissolved in 6 V toluene. K₃PO₄ (2.5 equiv) was added, and the slurry was stirred for 10 minutes at room temperature. Then trans-N,N-dimethylcyclohexane-1,2-diamine (0.5 equiv) and copper iodide (0.1 equiv) were added, and the reaction mass was heated to 95-100° C. The reaction was stirred at this temperature for 20 hours, after which time toluene was distilled. 5 V of methanol were added, and the reaction temperature was allowed to come up to 60-65° C. The reaction was stirred at this temperature for 2 hours. Methanol was distilled to 1 V, and then 6 V of water were added to precipitate a solid. The solid was filtered and washed with 2 V of water to yield 2.7 g (71.6%) of compound 093a.

### Example 23

Preparation of 4-chloro-2-(3-pyridyl) indazole (compound 093a)

2,6-Dichlorobenzaldehyde (100 g) and acetohydrazide (1.05 equiv) were added to a reaction flask equipped with column and Dean Stark trap and dissolved in toluene (10V). p-TSA (0.0005 equiv) was added to the solution, and the reaction was heated to 90-95° C. for 8 h with azeotropic distillation of water. The reaction mass was cooled to 40-45° C., and toluene was removed via vacuum distillation to ˜1V. Heptane (3 V) was added to precipitate a solid, and the slurry was stirred at 25-30° C. for 1 h. The solid N-[(E)-(2,6-dichlorophenyl)methyleneamino]acetamide (compound 197) was filtered and washed with 1V heptane and isolated in 87.5% yield with 99.36 area % purity according to HPLC analysis.

Compound 197 (5 g) was added to a reaction flask and dissolved in methanol (10 V). Sodium cyanoborohydride (2 equiv) and acetic acid (3V) were added, and the reaction was stirred at room temperature for 10 h. After reaction completion, water (20V) and toluene (10V) were added. The organic layer was separated, and the aqueous layer was washed with toluene (10V). The organic layers were combined, and toluene was removed in vacuo to ˜1V. Hexane (3V) was added to precipitate solid N′-[(2,6-dichlorophenyl)methyl]acetohydrazide (compound 040), which was filtered and washed with hexane (IV). Yield: 2.5 g (50%) with a purity of 98.07 area % according to HPLC analysis. Up to 70% of the material could be isolated by basifying the reaction mass to pH 8-9 with saturated Na₂CO₃ instead of doing a neutral extraction.

Compound 040) (0.5 g), 3-bromopyridine (1.0 equiv), trans-N,N′-dimethylcyclohexyldiamine (0.5 equiv) or N,N′-dimethylethylenediamine (0.5 equiv), copper iodide (0.3 equiv), and toluene were added to a reaction flask under nitrogen. The reaction mixture was heated to 105-110° C. for 24 h. In-process check at this time indicated ˜60 area % of 1-[4-chloro-2-(3-pyridyl)-3H-indazol-1-yl]ethanone (compound 082) and ˜10% of compound 093a

### Example 24

Preparation of compound 4-chloro-2-(3-pyridyl) indazole (compound 093a)

3-aminopyridine (20.0 g, 0.213 mol) was dissolved in 30 mL acetonitrile. Methanesulfonic acid (21.4 g, 0.223 mol) was added dropwise over a period of 5 minutes. The reaction was allowed to stir for 2 hours, at which point and a thick beige slurry had formed. The solid was isolated via filtration, washed with acetonitrile (2×5 mL) and dried in vacuo at room temperature to give 39.24 g of the methanesulfonic acid of 3-aminopyridine (compound 520a). Under nitrogen, the compound 520a (11.4 g, 0.060 mol) was suspended in sulfolane (115 mL). The slurry was heated to 60° C. 2-(bromomethyl)-1-chloro-3-nitrobenzene (compound 181a) (10.0 g, 0.040 mol) was charged, and the solution was heated until it reached a temperature of 110° C. The reaction was stirred for a total of ˜20 hours at this temperature. HPLC area percent analysis at a wavelength of 220 nm indicated the following reactant and product distribution: 21.5% compound 520a, 14.6% 2-(bromomethyl)-1-chloro-3-nitrobenzene (compound 182a), 47.2 percent of the protonated salt of the desired product N-[(2-chloro-6-nitrophenyl)methyl]pyridine-3-amine, 11.1% of 2-chloro-6-nitrophenyl) methanol (compound 050), and 3.7% of compound 181a.

Compound 182a (0.1 g 0.38 mmol) and zinc dust (0.100 g, 1.52 mmol) were weighed into a 20 mL scintillation vial equipped with a magnetic stirbar. Tetrahydrofuran (2 mL) was added, and the gray suspension was stirred at room temperature. Shortly after stirring was initiated, NaOH (0.152 g, 3.80 mmol) in 2 mL water was added dropwise, and the reaction was allowed to stir at room temperature for 4 h. The organic layer was sampled and analyzed by HPLC, indicating compound 093a had formed in 71 area % along with 29% of N-[(2-chloro-6-aminophenyl)methyl]pyridine-3-amine.

### Example 25

Screening of 3-aminopyridine salts (compound 520) for the synthesis of compound 182

Compound 181a (1.0 eq.), the appropriate 3-aminopyridine·HA salt (compound 520) (1.5 eq), and solvent were charged to a round bottom flask. The reaction mass was then heated to the listed temperature and were monitored by HPLC analysis for conversion. The results are reported in table 2 where “ND” refers to not detected, and “NR” refers to not reported

TABLE 2

| | | | | | | Pyridinium | | |
|---|---|---|---|---|---|---|---|---|
| | | | Temp | Time | 182a | byproduct | 050 | 181a |
| Entry | Solvent | HA | (° C.) | (h) | A % | A % | A % | A % |
| 1 | ACN | HCl | 82.5 | 22 | ND | ND | ND | NR |
| 2 | Sulfolane | HCl | 90-110 | 36 | 48.4 | 7.4 | 7.3 | 13.5 + 3.3 |
| 3 | ACN | H₂SO₄ | NR | NR | NR | NR | NR | NR |
| 4 | Sulfolane | H₂SO₄ | 90 | 22 | 2.8 | 68.1 | 2.8 | 0.3 |
| 5 | ACN | HCOOH | 82.5 | 2 | ND | 70.9 | 0.9 | ND |
| 6 | Sulfolane | HCOOH | NR | NR | NR | NR | NR | NR |
| 7 | ACN | MsOH | 82.5 | 12 | 60.3 | 6.4 | 8.9 | 9.8 |
| 8 | Sulfolane | MsOH | 110 | 12.5 | 47.2 | 14.5 | 11.1 | 3.6 |
| 9 | ACN | TsOH | 82.5 | 46 | 19.8 | 8.4 | 2.0 | 15.4 |
| 10 | Sulfolane | TsOH | 110 | 23 | 30.5 | 27.3 | 5.3 | 4.3 |

### Example 26

Preparation of N-[(2-chloro-6-nitro-phenyl)methyl]pyridin-3-amine (compound 182a)

1-(2-chloro-6-nitrophenyl)-N-(pyridine-3-yl) methanimine (compound 003a) (1 g, 3.82 mmol) was dissolved in 10 mL methanol. Sodium borohydride (0.145 g, 3.82 mmol) was added portionwise as a solid until bubbling ceased. The solution was then heated to 65° C. at reflux for 20 minutes and cooled to room temperature. Water (10 mL) was added to precipitate an orange solid, which was collected by vacuum filtration, washed with water (10 mL) and dried in vacuo at room temperature to yield 0.99 g of compound 182a (99%).

### Example 27

Preparation of 4-chloro-2-(3-pyridyl) indazole (compound 093a)

Charged 3-chloro-2-methyl-aniline (compound 114a) (1.0 equiv) and dichloroethane (20 V) to a flask under nitrogen. Charged a solution of potassium peroxymonosulfate (4.0 equiv) in water (80 V) as a single portion and the reaction was held at 25° C. until HPLC indicated completion of the reaction to 3-chloro-2-methyl-nitrosobenzene (compound 115a). The layers were separated and the dichloroethane solution of compound 116 (91.6 A % by HPLC) was taken forward to the next step.

Charged water (40 V) and bromine (1.7 equiv) to the dichloroethane solution of compound 115a in a vessel that was then sealed. The vessel was heated to 70° C. and irradiated using a 4 W white LED lamp. The reaction was held at 70° C. until HPLC indicated completion of the reaction to 3-chloro-2-(bromomethyl)-nitrosobenzene (compound 116b). The layers were separated and the dichloroethane solution of compound 116b (72.2 A % by HPLC) was taken forward.

Charged water (10 V) and 3-aminopyridinium chloride (compound 520b) (2.5 equiv) to the dichloroethane solution of compound 116b. The reaction was then heated to 70° C. and held overnight. Further water (10 V) and compound 520b (0.5 eq) were charged then the reaction was held at 70° C. until HPLC indicated completion of the reaction to 4-chloro-2-(3-pyridyl) indazole (compound 093a). The layers were separated and the dichloroethane solution of compound 093a exhibited 32.0 A % by HPLC.

### Example 28

Preparation of methyl 2-(pyridin-3-yl)-2H-indazole-4-carboxylate (compound 038)

The procedure from Example 27 was repeated starting from compound 400 (methyl 3-amino-2-methylbenzoate) to prepare compound 038 giving a dichloroethane solution of compound 038 (20.0 A % by HPLC).

### Example 29

Preparation of N-(1-methylcyclopropyl)-2-(3-pyridyl) indazole-4-carboxamide (compound 092)

Into a 100 mL HEL Hastelloy pressure reactor at room temperature were charged compound 093a (4-chloro-2-(3-pyridyl)indazole) (4.98 g, 21.6 mmol), 1,3-bis(cyclohexylphosphino)propane tetrafluoroborate (0.1336 g, 0.44 mmol), potassium carbonate (7.64 g, 55.9 mmol), and Pd/C wet (0.1278 g, 0.25 mol %). The reactor was then purged with nitrogen via pressurizing up to three bar and releasing three times. This was followed by injection of DMSO (50 mL), compound 070 (1-methylcyclopropanamine) (7.8 mL, 86.4 mmol) and pentane (48 mL). The reactor was heated to 60 C, and a vent line was opened in an attempt to purge the pentane/water azeotrope. The reactor system was closed and purged again with nitrogen three times followed by a carbon monoxide purge. The reactor was then pressurized up to ˜ 100-120 psi with carbon monoxide and heated to 110 degrees C. with agitation at 200 rpm. Agitation continued under these conditions for 20 h, then the reactor was cooled to room temperature and purged with nitrogen for IPC sampling. Analysis by UHPLC indicated the following area %: compound 061 (2-(3-pyridyl) indazole-4-carboxylic acid) (4.04 RRT, 7.8%), compound 092 (4.99 RRT, 85.6%), compound 093a (9.00 RRT, 0.56%). RRT=relative retention time.

### Example 30

The above procedure was repeated using compound 093b (4-bromo-2-(3-pyridyl) indazole) using Pd/C (0.15 mol %). Analysis by UHPLC indicated the following area percent for the reaction mixture: compound 061 (2-(3-pyridyl) indazole-4-carboxylic acid) (4.04 RRT, 8.3%), compound 092 (4.99 RRT, 87.9%). RRT=relative retention time.

### Example 31

Preparation of N-(1-methylcyclopropyl)-2-(3-pyridyl) indazole-4-carboxamide (compound 092)

Into a 600 mL Parr pressure reactor at room temperature were charged compound 093a (4-chloro-2-(3-pyridyl)indazole) (41.35 g, 180.0 mmol), 1,3-bis(cyclohexylphosphino)propane tetrafluoroborate (0.661 g, 1.08 mmol), potassium phosphate tribasic (43.96 g, 207.05 mmol), and Pd/C dry (0.575 g, 0.30 mol %). The reactor was then purged with nitrogen via pressurizing up to three bar and releasing three times. This was followed by injection of DMSO (300 mL) and compound 070 (13.44 g, 189.04 mmol). The reactor was then pressurized up to 75 psi with carbon monoxide and heated to 110° C. with agitation at 200 rpm. Agitation continued under these conditions for 8 h, then the reactor was cooled to room temperature and purged with nitrogen for IPC sampling. Analysis by UHPLC indicated the following area percents: compound 061 (2-(3-pyridyl) indazole-4-carboxylic acid) (4.04 RRT, 5.4%), compound 092 (4.99 RRT, 91.8%), compound 093a (9.00 RRT, 0.30%). RRT=relative retention time. Typical reaction yield is ˜ 78% after filtration of the catalyst residue and recrystallization from DMSO with water as the antisolvent.

### Example 32

### Part A.

Compound 093a (4.6 g, 1.0 eq), 10 wt % Pd/C, 1,3-bis(cyclohexylphosphino) propane tetrafluoroborate (Pd:Ligand 2:1), base(s), compound 070 (4 eq.), and DMSO (10-11V) were charged into a reactor that was purged with nitrogen then pressurized with CO (100 psi). The reaction mass was then heated to 110° C. and agitated for 20 hr. The reaction mass was then analyzed using HPLC Area % analysis and the results are reported in table 3.

TABLE 3

| | | | Ligand | Conv., | 061 | 092 | Other |
|---|---|---|---|---|---|---|---|
| Base | Base eq. | Pd mol % | mol % | area % | Area % | Area % | Area % |
| Bu3N | 1.5 | 0.21% | 0.52% | 19.5% | 18.50% | 37.1% | 41.3% |
| Et3N | 3 | 0.41% | 0.82% | 33.4% | 7.80% | 45.6% | 43.9% |
| t-BuOK | 1.5 | 0.21% | 0.52% | 100.0% | 4.40% | 0.0% | 96.0% |
| KOAc | 1.5 | 0.21% | 0.52% | 58.7% | 45.10% | 26.6% | 25.6% |
| Al(OH)3 | 1.5 | 0.21% | 0.52% | 59.6% | 8.00% | 32.6% | 55.0% |
| Cs2CO3 | 1.5 | 0.21% | 0.52% | 95.0% | 86.40% | 11.9% | 1.4% |
| Na3PO4 | 1.5 | 0.21% | 0.52% | 43.7% | 15.60% | 59.3% | 19.1% |
| Na2HPO4 | 1.5 | 0.21% | 0.52% | 44.2% | 6.50% | 41.5% | 48.0% |
| Na3PO4 and | 2 | 0.40% | 0.80% | 99.0% | 26.90% | 72.9% | 0.3% |
| Na2HPO4 | | | | | | | |
| (3:1) | | | | | | | |
| K3PO4 | 1.5 | 0.40% | 0.82% | 99.6% | 16.00% | 77.5% | 2.7% |
| K3PO4 and | 2 | 0.40% | 0.80% | 99.7% | 13.40% | 74.9% | 11.4% |
| K2HPO4 | | | | | | | |
| (3:1) | | | | | | | |
| K3PO4 and | 2 | 0.20% | 0.40% | 89.7% | 15.90% | 75.1% | 8.5% |
| K2HPO4 | | | | | | | |
| (3:1) | | | | | | | |

Part. B

Compound 093a (4.6 g, 1.0 eq), 10 wt % Pd/C (0.41 mol %), 1,3-bis(cyclohexylphosphino) propane tetrafluoroborate (Pd:Ligand 2:1), K₃PO₄:K₂HPO₄ (3:1, 2 eq.), compound 070 (4 eq.), and solvent (10 V) were charged into a reactor that was purged with nitrogen then pressurized with CO (100 psi). The reaction mass was then heated to 110° C. and agitated for 20 hr. The reaction mass was then analyzed using HPLC Area % analysis and the results are reported in table 4.

TABLE 4

| | Conv. | 061 | 092 | Other |
|---|---|---|---|---|
| Solvent | Area % | Area % | Area % | Area % |
| Diglyme | 19.7% | 58.5% | 8.6% | 32.9% |
| Dioxane | 16.9% | 58.3% | 23.2% | 20.0% |
| acetonitrile | 83.6% | 24.4% | 54.2% | 22.3% |
| DMF | 72.6% | 14.1% | 63.8% | 23.1% |
| DMAc | 32.4% | 37.3% | 8.6% | 55.2% |
| Sulfolane | 99.8% | 7.9% | 55.3% | 50.8% |
| DMSO | 99.7% | 6.9% | 85.3% | 7.3% |

### Example 33: Effect of CO Partial Pressure on the Rate of Consumption of Compound 093 in the Aminocarbonylation Reaction to Form Compound 092

Compound 093 (5.74 g, 1.0 eq), Pd (0.3 mol %) charged as 10 wt % Pd on Carbon, 1,3-bis(cyclohexylphosphino)propane tetrafluoroborate (Ligand:Pd 2:1), H₃PO₄ base (1.2 eq.), compound 070 (1.5 eq.), and DMSO (7.3V) were charged into a reactor that was purged with nitrogen, purged with CO, then pressurized with CO to the desired pressure. The reaction mass was then heated to 110° C. and agitated for 20 hr. The CO gas uptake was measured to determine the rate and conversion of each reaction as a function of time. The amount of CO consumed and reaction conversion at 5.1 hr is shown for each reaction in Table 5. The final reaction mass at 20 hr was then analyzed using HPLC Area % analysis, and the results are reported in Table 6. The data show that reaction selectivity is approximately the same at full conversion regardless of CO pressure or rate.

TABLE 5
CO Consumed and Reaction Conversion at 5.1 hr

| CO Pressure, | CO Consumed, | Reaction |
|---|---|---|
| psig | mmol | Conversion, % |
| 25 | 24.6 | 98.4% |
| 50 | 17.7 | 70.8% |
| 100 | 14.8 | 59.2% |
| 180 | 7.0 | 28.0% |

TABLE 6
Conversion and Selectivity at 20 hr

| CO Pressure, | Conv., | 061 | 092 | Other |
|---|---|---|---|---|
| psig | area % | Area % | Area % | Area % |
| 25 | 99.7% | 9.2% | 88.9% | 1.9% |
| 50 | 99.7% | 7.3% | 90.9% | 1.8% |
| 100 | 99.4% | 8.4% | 89.7% | 1.9% |
| 180 | 90.0% | 12.2% | 85.1% | 2.7% |

### Example 34

Preparation of methyl 2-(3-pyridyl) indazole-4-carboxylate (compound 038)

Into a 100 mL HEL Hastelloy pressure reactor at room temperature were charged compound 093a (4-chloro-2-93-pyridyl) indazole) (4.00 g, 0.015 mol), 1,3-bis(cyclohexylphosphino) propane tetrafluoroborate (0.106 g, 0.17 mmol), sodium phosphate (4.28 g, 0.026 mol), and palladium acetate (0.0195 g, 0.087 mmol). The reactor was then purged with nitrogen via pressurizing up to three bar and releasing three times. This was followed by injection of xylenes (36.4 mL) and methanol (8.5 mL). The reactor system was closed and purged again with nitrogen three times followed by a carbon monoxide purge. The reactor was then pressurized up to ˜60 psi with carbon monoxide and heated to 140 degrees C. with agitation at 800 rpm. The gauge pressure was ˜ 75 psi. Agitation continued under these conditions for 20 h, then the reactor was cooled to room temperature and purged with nitrogen for IPC sampling. Analysis by UHPLC indicated the following area percents: compound 061 (2-(3-pyridyl) indazole-4-carboxylic acid) (4.04 RRT, 5.6%), compound 038 (7.06 RRT, 91.3%), compound 093a (4-chloro-2-(3-pyridyl) indazole) (9.00 RRT, 0.3%). RRT=relative retention time.

### Example 35

Preparation of methyl 2-(3-pyridyl) indazole-4-carboxylate (compound 038) and in situ conversion to 2-(3-pyridyl) indazole-4-carboxylic acid (compound 061)

Into a dry reactor under nitrogen was charged 4-bromo-2-(3-pyridyl)-indazole (compound 93b) (25.59 kg, 1.0 eq), o-xylene (6 V), methanol (1.5 V), and triethylamine (1.01 V) then the reaction mass was degassed with nitrogen. Then palladium acetate (2.0 mol %) and 4,5-Bis(diphenylphosphino)-9,9-dimethylxanthene (4 mol %) was charged and the reaction was further degassed with nitrogen. The reactor was then pressurized with CO (60-70 psi) and heated to 75° C. until completion of the reaction to compound 038 according to HPLC analysis. Analysis by HPLC indicated the following A %: compound 93 (0.22 A %), compound 38 (95.55 A %), and compound 061 (0.64 A %). The reaction mass was then returned to atmospheric pressure and purged with nitrogen. Aqueous NaOH (3 N, 9 V) was added then the reaction mass was heated to 80° C. until completion of the reaction to compound 061 was confirmed by HPLC analysis. Analysis by HPLC indicated the following A %: compound 061 (95.04 A %). Activated charcoal (2.95 kg) was charged to the reaction mass, the reaction was filtered through a celite bed (14.79 kg) and the celite bed was washed with water (5.0 V). The organic and aqueous layers were separated and the aqueous layer was washed with a mixture of ethyl acetate (5 V) and toluene (10 V). The aqueous layer was acidified to pH 3 using concentrated hydrochloric acid then further water (5 V) was charged. The aqueous layer was then filtered and the solid was dried to give compound 61 (14.0 kg). Further compound 061 was recovered from the organic layer and the celite bed to give a total of 20.5 kg of compound 061.

### Example 36

Preparation of methyl 2-(pyridyl)-indazole-4-carboxylate (compound 038)

Charged 2-(3-pyridyl) indazole-4-carboxylic acid (compound 061) (7 g) and methanol (14.3 V) to a flask under nitrogen. Sulfuric acid (3.0 eq.) was charged dropwise over 2 min. The reaction was then heated to 60° C. and held at that temperature until HPLC indicated completion of the activation. The reaction was then cooled to 0° C. An aqueous sodium bicarbonate solution (194 mL, 5 wt %) was then added dropwise over 20 min. The slurry was then filtered and the filter cake was washed with the filtrate. The solid was dried at 90° C. under vacuum to yield crude compound 038 as a tan solid (10 g, 95.6 A %).

### Example 37

Preparation of N-(1-methylcyclopropyl)-2-(3-pyridinyl)-2H-indazole 4-carboxamide (compound 092)

Charged 2-(3-pyridyl) indazole-4-carboxylic acid (5.0 g) (compound 061), tosyl chloride (1.2 eq.), and acetonitrile (15 V) into a jacketed reactor with stirring under nitrogen. The reaction mass was heated to 80° C. Then charged N-methylimidazole (2.0 eq) slowly into the reaction mass over 5 min. The reaction was held at 80° C. until HPLC indicated completion of the activation. Then 1-methylcyclopropaneamine (compound 070) (1.2 eq) was charged slowly into the reaction mass over 5 min. The reaction was held at 80° C. until HPLC indicated completion of the reaction to compound 092. Then water (10 V) was added slowly over 10 min and the reaction mass was heated until it returned to 80° C. The slurry was then filtered, washed with the filtrate, and washed with water (2×10 V). The solid was dried at 60° C. under vacuum to yield compound 092 as an off-white solid (4.52 g, 95.1 wt %, 70.3% yield)

### Example 38

Preparation of N-(1-methylcyclopropyl)-2-(3-pyridinyl)-2H-indazole 4-carboxamide (compound 092)

Charged methyl 2-(pyridin-3-yl)-2H-indazole-4-carboxylate (compound 038) (200 mg), THF (10 V), and 1-methylcyclopropaneamine (compound 070) (2.5 eq.) to a dry flask with stirring under nitrogen. The reaction mass was cooled to 0° C. Then isopropyl magnesium chloride (2.0 eq.) was charged slowly over 5 min, keeping the reaction temperature below 10° C. The reaction was then held at 0° C. for 4 hrs. Then water (5 V) was added slowly over 5 minutes after which additional water (15 V) was added and the reaction mass was stirred at 23° C. for 10 min. The slurry was filtered and washed with water (3×15 V). The solid was dried at 80° C. under vacuum to yield compound 092 as a light-yellow solid (200 mg, 98 A %, 86.6% yield)

### Example 39

Preparation of N-(1-methylcyclopropyl)-2-(3-pyridinyl)-2H-indazole 4-carboxamide (compound 092)

Part A. Charged 2-(3-pyridyl) indazole-4-carboxylic acid (25 g) (compound 061), toluene (5V), and catalyst (0.05 eq.) into a jacketed reactor with stirring under nitrogen. Thionyl chloride (1.25 eq.) was charged dropwise into the reaction mass. After addition was complete, the reaction mass was heated to 85-90° C. and held until the reaction was complete by HPLC. Toluene (2V) was distilled off at 85-90° C. under vacuum (˜300 torr). The reaction mass was cooled to 40° C. and fresh toluene (3V) was charged into the reaction mass. In some cases, N-methyl-2-pyrrolidone (1V) was charged in replacement of 1V toluene.

Part B. A solution of 1-methylcyclopropanamine (1.2 eq.) (compound 070) in toluene (2V) was charged dropwise into the reaction mass from Part A and stirred for 20 mins. Then the base was charged dropwise into the reaction mass, followed by heating the reaction mass to 60-65° C. The reaction was held at this temperature until the reaction was indicated complete by HPLC analysis. The yield of N-(1-methylcyclopropyl)-2-(3-pyridinyl)-2H-indazole 4-carboxamide (compound 092) was determined by HPLC wt % analysis and is shown in Table 7.

TABLE 7

| | | Toluene | NMP | | Base | Compound |
|---|---|---|---|---|---|---|
| Exp | Catalyst | (V) | (V) | Base | (Eq.) | 092 yield (%) |
| 1 | DMF | 7 | 1 | TEA | 1.7 | 89 |
| 2 | DMF | 7 | 1 | Pyridine | 3.2 | 89 |
| 3 | DMF | 7 | 1 | DIPEA | 2.2 | 93 |
| 4 | DMF | 8 | 0 | DIPEA | 2.2 | 93 |
| 5 | Pyridine | 8 | 0 | DIPEA | 2.2 | 93 |

### Example 40

Preparation of N-(1-methylcyclopropyl)-2-(3-pyridinyl)-2H-indazole 4-carboxamide (compound 092) Form A

Part A. Charged 2-(3-pyridyl) indazole-4-carboxylic acid (100 g) (compound 061), toluene (5V), and N,N-dimethylformamide (0.05 eq.) into a jacketed reactor with stirring under nitrogen. Thionyl chloride (2 eq.) was charged dropwise into the reaction mass. After addition was complete, the reaction mass was heated to 70-75° C. and held until the reaction was complete by HPLC. The reaction mass was cooled to 40-50° C. and toluene was distilled off under vacuum until 1V remained. Additional toluene (2V) was charged and distilled to 1V to remove any remaining thionyl chloride. Heptane (2V) was charged and distilled until 1V remained. The reaction mass was cooled to room temperature and additional heptane (5V) was charged. The slurry was filtered under a N₂ blanket and the acid chloride reaction product (compound 930) wet cake was used directly in the next step.

Part B. Charged the acid chloride from Part A, acetonitrile (14V), and N-methylpyrrolidone (1V) into a jacketed reactor with stirring under nitrogen. Then charged 1-methylcyclopropanamine (1.2 eq.) (compound 070) slowly into the reaction mass. Lastly, charged triethylamine (2.1 eq.) slowly into the reaction mass, maintaining the temperature <40° C. The reaction mass was heated to 60-65° C. and held until HPLC indicated completion of the reaction to compound 092. Acetonitrile was distilled off at <50° C. to ˜3V under vacuum. The reaction mass was cooled to room temperature and N-methylpyrrolidone (2V) was charged. The reaction mass was heated to 95-100° C. and held for 10 mins, cooled to 90° C. and held for 15 mins, then 3 wt % N-(1-methylcyclopropyl)-2-(3-pyridinyl)-2H-indazole 4-carboxamide (compound 092) seed was charged. Water (2.5V) was charged at 90° C. over 3 hours. The slurry was cooled to 20° C. over 5 hours, stirred for 20 mins, filtered, and washed with water (4.5V). The solid was dried at 60° C. to yield N-(1-methylcyclopropyl)-2-(3-pyridinyl)-2H-indazole 4-carboxamide (compound 092) as an off-white solid (106.5 g, 97.4 wt %, 84.9% yield). The solid was identified as polymorph Form A.

### Example 41

Preparation of N-(1-methylcyclopropyl)-2-(3-pyridinyl)-2H-indazole 4-carboxamide (compound 092) Form B

Part A. Charged 2-(3-pyridyl) indazole-4-carboxylic acid (15 g) (compound 061), toluene (15V), and N,N-dimethylformamide (0.1V) into a 250 mL multi-neck round-bottom flask with stirring under nitrogen. Thionyl chloride (2 eq.) was charged slowly into the reaction mass. After addition was complete, the reaction mass was heated to 70-75° C. and held until the reaction was complete by HPLC. The reaction mass was cooled to 40-50° C. and toluene was distilled off under vacuum. Fresh toluene (3V) was charged and distilled again. A third charge of fresh toluene (3V) was added and distilled off. The reaction mass was cooled to 40-45° C. and N-methylpyrrolidone (1V) was charged. The reaction mass was distilled to remove most of the remaining toluene. The reaction mass was then cooled to 25-30° C. and acetonitrile (14V) was charged, followed by slow addition of 1-methylcyclopropanamine (1.2 eq.) (compound 070) into the reaction mass. Lastly, triethylamine (2 eq.) was charged slowly into the reaction mass. The reaction mass was heated to 60-65° C. and held until HPLC indicated completion of the reaction to compound 092. Acetonitrile was distilled off at <50° C. to ˜3V under vacuum. The reaction mass was cooled to 25-30° C. An aqueous 1% NaOH solution (10V) was charged into the reaction mass and stirred for 1 hr. The reaction mass was filtered to collect the solid and washed with water (5V). The solid was dried under vacuum at 45-50° C. to yield N-(1-methylcyclopropyl)-2-(3-pyridinyl)-2H-indazole 4-carboxamide (compound 092) as an off-white solid (14.5 g, 98.8 wt %, 78.3% yield). The solid was identified as polymorph Form B.

### Example 42

Preparation and Analysis of the Crystalline Compound 092 Form A by single-crystal X-ray diffraction

Solids of compound 092 (109.1 mg) were combined with EtOH (4 mL) with stirring at about 72° C., and the resulting slurry was left to stir at about 72-73° C. After about 1 day, the bulk of solids in the sample were isolated by positive-pressure filtration. Some solids remained in the original vial following filtration, and from these a single crystal was culled and analyzed. A colorless needle having approximate dimensions of 0.55×0.04× 0.03 mm³, was mounted on a polymer loop in random orientation. Preliminary examination and data collection were performed on a Rigaku SuperNova diffractometer, equipped with a copper anode microfocus sealed X-ray tube (Cu Kα λ=1.54184 Å) and a Dectris Pilatus3 R 200K hybrid pixel array detector. Form A is an anhydrous/non-solvated form of compound 092. The crystal data and data collection parameters for compound 092 Form A are shown in the table 8.

TABLE 8

| Empirical formula | | C₁₇H₁₆N₄O |
|---|---|---|
| Formula weight (g mol⁻¹) | | 292.34 |
| Temperature (K) | | 299.8(4) |
| Wavelength (Å) | | 1.54184 |
| Crystal system | | monoclinic |
| Space group | | P2₁ |
| Unit cell parameters | | |
| a = 11.2344(3) | Å | α = 90° |
| b = 4.98046(11) | Å | β = 91.803(2)° |
| c = 13.0556(3) | Å | γ = 90° |

| | |
|---|---|
| Unit cell volume (Å³) | 730.13(3) |
| Cell formula units, Z | 2 |
| Calculated density (g cm⁻³) | 1.330 |
| Absorption coefficient (mm⁻¹) | 0.694 |
| F(000) | 308 |
| Crystal size (mm³) | 0.55 × 0.04 × 0.03 |
| Reflections used for cell measurement | 1774 |
| ϑ range for cell measurement | 3.9240°-77.2770° |
| Total reflections collected | 3167 |
| Index ranges | −14 ≤ h ≤ 14; −4 ≤ k ≤ |
| | 6; −16 ≤ l ≤ 15 |
| ϑ range for data collection | ϑₘᵢₙ = 3.387°, ϑₘₐₓ = 77.736° |
| Completeness to θₘₐₓ | 94.5% |
| Completeness to θ_{full} = 67.684° | 99.7% |
| Absorption correction | multi-scan |
| Transmission coefficient range | 0.829-1.000 |
| Transmission coefficient range | 0.829-1.000 |
| Refinement method | full matrix least-squares on F² |
| Independent reflections | 2044 [Rᵢₙₜ = 0.0296, R_{σ} = |
| | 0.0499] |
| Reflections [I > 2σ(I)] | 1809 |
| Reflections/restraints/parameters | 2044/1/263 |
| Goodness-of-fit on F² | S = 1.01 |
| Final residuals [I > 2σ(I)] | R = 0.0381, R_{w} = 0.0971 |
| Final residuals [all reflections] | R = 0.0442, R_{w} = 0.1012 |
| Largest diff. peak and hole (e Å⁻³) | 0.137, −0.178 |
| Max/mean shift/standard uncertainty | 0.000/0.000 |

### Example 43

Preparation and Analysis of the Crystalline Compound 092 Form B by single-crystal X-ray diffraction

Solids of compound 092 (102.6 mg) were combined with ACN (4 mL) with stirring at about 72-73° C., and the resulting slurry was left to stir at about 72-73° C. After 1 day, the solids in the sample were isolated by positive-pressure filtration, and the warm filtrate was placed directly on the lab bench to cool to RT. After standing at RT for 6 days, a clear liquid and white solids consisting of spherulites of thin needles were observed. A needle was culled and analyzed. A colorless needle having approximate dimensions of 0.31×0.03× 0.02 mm³, was mounted on a polymer loop in random orientation. Preliminary examination and data collection were performed on a Rigaku SuperNova diffractometer, equipped with a copper anode microfocus sealed X-ray tube (Cu Ka1=1.54184 Å) and a Dectris Pilatus3 R 200K hybrid pixel array detector. Form B is an anhydrous/non-solvated form of compound 092. The crystal data and data collection parameters for compound 092 Form B are shown in the table 9.

TABLE 9

| Empirical formula | | C₁₇H₁₆N₄O |
|---|---|---|
| Formula weight (g mol⁻¹) | | 292.34 |
| Temperature (K) | | 299.6(4) |
| Wavelength (Å) | | 1.54184 |
| Crystal system | | orthorhombic |
| Space group | | P2₁2₁2₁ |
| Unit cell parameters | | |
| a = 5.05866(12) | Å | α = 90° |
| b = 11.2486(3) | Å | β = 90° |

| | |
|---|---|
| c = 25.5418(8) | γ = 90° |
| Unit cell volume (Å³) | 1453.40(7) |
| Cell formula units, Z | 4 |
| Calculated density (g cm⁻³) | 1.336 |
| Absorption coefficient (mm⁻¹) | 0.698 |
| F(000) | 616 |
| Crystal size (mm³) | 0.31 × 0.03 × 0.02 |
| Reflections used for cell measurement | 2011 |
| ϑ range for cell measurement | 4.2980°-74.9370° |
| Total reflections collected | 4550 |
| Index ranges | −6 ≤ h ≤ 6; −14 ≤ k ≤ |
| | 14; −31 ≤ l ≤ 15 |
| ϑ range for data collection | ϑₘᵢₙ = 4.295°, ϑₘₐₓ = 77.364° |
| Completeness to θₘₐₓ | 96.8% |
| Completeness to θ_{full} = 67.684° | 99.8% |
| Absorption correction | multi-scan |
| Transmission coefficient range | 0.780-1.000 |
| Refinement method | full matrix least-squares on F² |
| Independent reflections | 2683 [Rᵢₙₜ = 0.0280, R_{σ} = |
| | 0.0450] |
| Reflections [I > 2σ(I)] | 2171 |
| Reflections/restraints/parameters | 2683/0/263 |
| Goodness-of-fit on F² | S = 1.04 |
| Final residuals [I > 2σ(I)] | R = 0.0373, R_{w} = 0.0845 |
| Final residuals [all reflections] | R = 0.0514, R_{w} = 0.0916 |
| Largest diff. peak and hole (e Å⁻³) | 0.150, −0.186 |
| Max/mean shift/standard uncertainty | 0.000/0.000 |

### Example 44

X-Ray Powder Diffraction characterization of Compound 092 for polymorphs form identification

Powder X-ray diffraction was used to identify the crystalline phases of various samples of Compound 092. X-ray diffraction patterns were collected with a PANalytical X′Pert PRO MPD or a PANalytical Empyrean diffractometer using an incident beam of Cu radiation produced using an Optix long, fine-focus source. An elliptically graded multilayer mirror was used to focus Cu Kα X-rays through the specimen and onto the detector. Prior to the analysis, a silicon specimen (NIST SRM 640e) was analyzed to verify the observed position of the Si 111 peak is consistent with the NIST-certified position. A specimen of the sample was sandwiched between 3-μm-thick films and analyzed in transmission geometry. A beam-stop, short antiscatter extension, and antiscatter knife edge were used to minimize the background generated by air. Soller slits for the incident and diffracted beams were used to minimize broadening from axial divergence. Diffraction patterns were collected using a scanning position-sensitive detector (X′Celerator) located 240 mm from the specimen and Data Collector software v. 5.5.

Diffraction maxima were identified from the X-ray diffractograms generated by the software. A characteristic subset of the 20 diffraction maxima for polymorph A of Compound 092 is given in Table 1. A characteristic subset of the 20 diffraction maxima for polymorph B of Compound 092 is given in Table 2. As the X-ray diffraction patterns for polymorph A and polymorph B of Compound 092 are distinctly different from each other, the polymorph form of a sample of unknown polymorph form could be easily determined by comparing its characteristic 20 X-ray maxima to the characteristic 20 maxima shown in Tables 1 and 2, respectively.

The X-ray powder diffraction (XRPD) diagram for compound 092 Form A is shown in FIG. 1. The XRPD diagram for compound 092 Form B is shown in FIG. 2.

TABLE 10
Characteristic 2θ X-ray Maxima (in degrees)
for Polymorph Form A of Compound 092
2θ

7.9
13.6
15.5
17.4
20.4
21.7
22.4
27.2
28.2
34.0

TABLE 11
Characteristic 2θ X-ray Maxima (in degrees)
for Polymorph Form B of Compound 092
2θ

8.6
13.1
13.9
16.0
17.2
20.5
20.9
22.4
26.0
29.1

### Example 45

### Stable Polymorph Form Screen for Compound 092

Slurry experiments were conducted to identify the stable form at various conditions. In these experiments, saturated solutions in various solvents containing excess undissolved solids (starting with Form A+minor Form B) were stirred for extended durations. Slurries at room temperature (RT) and 2-8° C. were typically conducted for approximately 2 weeks, while slurries at 72-73° C. were stirred for a shorter duration (1 day) to minimize the potential for decomposition. The Form B component converted to Form A in nearly all slurries, indicating that Form A is more stable than Form B between 2° C. and 73° C. The results are shown in the table 12 where the time and temperatures are approximate.

TABLE 12

| | | | Final form |
|---|---|---|---|
| Solvent | Conditions | Observations | (XRPD Result) |
| acetone | RT, 13 d | opaque off-white suspension; | Form A |
| | | fines | (analyzed |
| | | | damp) |
| ACN | 72-73° C., | opaque off-white suspension, | Form A + |
| | 1 d | light brown and off-white | minor |
| | | solids on walls above soln.; | Form B |
| | | fines and agglomerates, B/E | |
| DCM | RT, 13 d | opaque off-white suspension; | Form A |
| | | fines | (analyzed |
| | | | damp) |
| DMF | RT, 13 d | opaque off-white suspension; | Form A |
| | | fines | (analyzed |
| | | | damp) |
| EtOH | RT, 13 d | opaque off-white suspension; | Form A |
| | | fines | (analyzed |
| | | | damp) |
| | 72-73° C., | clear soln., off-white and | Form A |
| | 1 d | light brown solids on walls | |
| | | above soln.; thin needles and | |
| | | agglomerates | |
| IPA | RT, 13 d | opaque off-white suspension; | Form A |
| | | fines | (analyzed |
| | | | damp) |
| | 72-73° C., | slightly hazy soln., light | Form A + |
| | 1 d | brown solids on walls above | very minor |
| | | soln.; small needles, | Form B |
| | | agglomerates, and fines | |
| MEK | RT, 13 d | opaque off-white suspension; | Form A |
| | | fines | (analyzed |
| | | | damp) |
| | 72-73° C., | opaque off-white suspension; | Form A |
| | 1 d | tiny needles | |
| MeOH | RT, 13 d | opaque off-white suspension; | Form A |
| | | fines | (analyzed |
| | | | damp) |
| | 2-8° C., | opaque off-white suspension; | Form A |
| | 14 d | fines | |

### Example 46

### Compound 092 Form A and Form B Interconversion Study

Relative thermodynamic stabilities of anhydrous/non-solvated Forms A and B were studied at 2-8° C., room temperature (RT) and at 71-72° C. via interconversion (competitive) slurries. In these slurries, a given solvent system was pre-saturated with compound 092 (Form A+minor Form B) at the stated temperature, and a portion of the liquid phase was filtered into a mixture of solids containing relatively equal amounts of Form A and Form B. Interconversion slurries at 2-8° C. and RT were allowed to stir for 22 days, while slurries at 71-72° C. stirred for shorter duration (2-4 days). Almost all of the competitive slurries exhibited conversion to Form A, indicating that Form A is the thermodynamically stable form between 2° C. and 72° C. The results are reported in table 13 below where: liquid phase for each slurry was pre-saturated using as-received Form A+minor Form B; solvent ratios are v/v; water activity values were calculated using UNIFAC calculator at RT; and times and temperatures are approximate.

TABLE 13

| Starting | | | | Final form |
|---|---|---|---|---|
| Materials/ | | | | (XRPD |
| Forms | Solvent | Conditions | Observations | Result) |
| Form A + | THF | 2-8° C., | opaque off-white | Form A + |
| Form B | | 22 d | suspension; fin | Form B |
| | | RT, 22 d | opaque off-white | Form A + |
| | | | suspension; fines | very minor |
| | | | | Form B |
| | EtOH | RT, 22 d | opaque off-white | Form A |
| | | | suspension; fines | |
| | | 71-72° C., | opaque tan | Form A |
| | | 2 d | suspension; fines | |
| | | | and needles | |
| | NMP/water | 2-8° C., | opaque off-white | Form A |
| | 80:20 | 22 d | suspension; fines | |
| | (a_{w} 0.39) | RT, 22 d | opaque off-white | Form A |
| | | | suspension; fines | |
| | | 71-72° C., | opaque tan | Form A |
| | | 2 d | suspension; fines | (analyzed |
| | | | and needles | damp) |
| | | | (isolated as a | |
| | | | damp paste) | |
| | NMP/water | RT, 22 d | opaque off-white | Form A + |
| | 3:97 | | suspension; fines | Form B |
| | (a_{w} 0.99) | 71-72° C., | opaque off-white | Form A + |
| | | 4 d | suspension; fines | minor Form B |

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

It is to be noted that the term “a” or “an” entity refers to one or more of that entity; for example, “a solvent system” is understood to represent one or more solvent systems. As such, the terms “a”, “an”, “one or more”, and “at least one” can be used interchangeably herein.

Efforts have been made to ensure accuracy with respect to numbers used (such as without limitation, temperature, concentration, etc.), but some experimental errors and deviations may be accounted for.

Throughout this specification and the claims, the words “comprise”, “comprises”, and “comprising” are used in a non-exclusive sense, except where the context requires otherwise. It is understood that embodiments and aspects described herein include “predominantly comprising”, “consisting of” and/or “consisting essentially of” embodiments and aspects.

As used herein, the word “predominantly” means greater than 50%, at least 75%, at least 90%, at least 95%, or at least 99% on a relevant basis such as, for instance and without limitation, population %, w/w %, w/v %, v/v %, and area %.

Where a range of values is provided, it is understood that each intervening value, to the tenth of the unit of the lower limit, unless the context clearly dictates otherwise, between the upper and lower limit of the range and any other stated or intervening value in that stated range, is encompassed herein. The upper and lower limits of these small ranges which can independently be included within the smaller ranges is also encompassed herein, subject to any specifically excluded limit in the stated range. Where the stated range includes one or both of the limits, ranges excluding either or both of those include limits are also included herein.

Many modifications and other embodiments and aspects of the invention set forth herein may come to mind to one skilled in the art to which these inventions pertain having the benefit of the teachings presented in the foregoing descriptions. Therefore, it is to be understood that the inventions are not limited to the specific embodiments and aspects disclosed and that modifications and other embodiments and aspects are intended to be within the scope of the claims. Although specific terms may be employed herein, they are used in a generic and descriptive sense only and not for purposes of limitation.

1. A process for the preparation of compound 093a, the process comprising:
(1) forming compound 181a by step 1 comprising forming a reaction mixture comprising compound 339, a source of Br, optionally a photo- or thermally sensitive radical initiator, and a solvent system, and reacting the reaction mixture under photochemical conditions to form a reaction product mixture comprising compound 181a according to the following reaction scheme 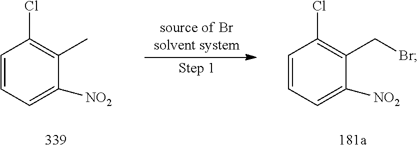
(2) forming compound 378 by the (i) the combination of steps 2(a) and 2(b) or by (ii) step 2,
(i) wherein step 2(a) comprises forming a reaction mixture comprising compound 181a, a base, and a solvent system, and reacting the reaction mixture to form a reaction product mixture comprising compound 050 according to the following reaction scheme 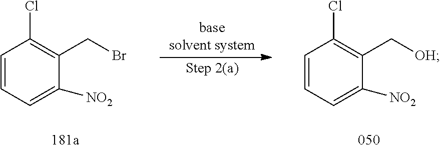 [SMILES: O=[N+]([O-])c1cccc(Cl)c1CBr ; O=[N+]([O-])c1cccc(Cl)c1CO]
and
wherein step 2(b) comprises forming a reaction mixture comprising compound 050, an oxidizing reagent, a phase transfer catalyst, and a solvent system, and reacting the reaction mixture to form a reaction product mixture comprising compound 378 according to the following reaction scheme 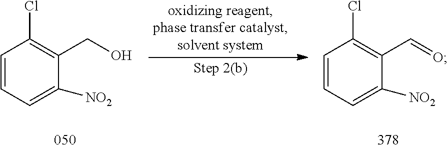 [SMILES: O=[N+]([O-])c1cccc(Cl)c1CO ; O=Cc1c(Cl)cccc1[N+](=O)[O-]]
and
(ii) wherein step 2 comprises forming a reaction mixture comprising compound 181a, an oxidizing reagent, and a solvent system, and reacting the reaction mixture to form a reaction product mixture comprising compound 378 according to the following reaction scheme 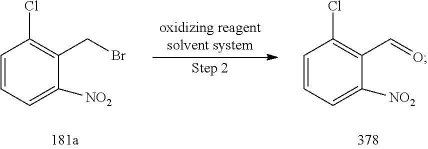 [SMILES: O=[N+]([O-])c1cccc(Cl)c1CBr ; O=Cc1c(Cl)cccc1[N+](=O)[O-]]
(3) forming compound 003a by step 3 comprising forming a reaction mixture comprising compound 378, 3-aminopyridine, an acid catalyst, and a solvent system, and reacting the reaction mixture to form a reaction product mixture comprising compound 003a according to the following 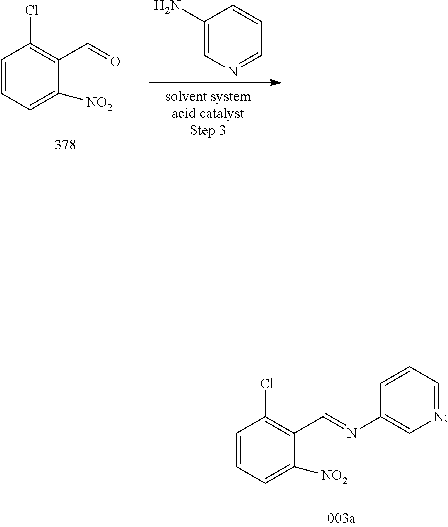 [SMILES: O=Cc1c(Cl)cccc1[N+](=O)[O-] ; Nc1cccnc1 ; O=[N+]([O-])c1cccc(Cl)c1/C=N/c1cccnc1]
and
(4) forming compound 093a by step 4 comprising forming a reaction mixture comprising compound 003a, a phosphine or a phosphite, and a solvent system, and reacting the reaction mixture to form a reaction product mixture comprising compound 093a according to the following reaction scheme 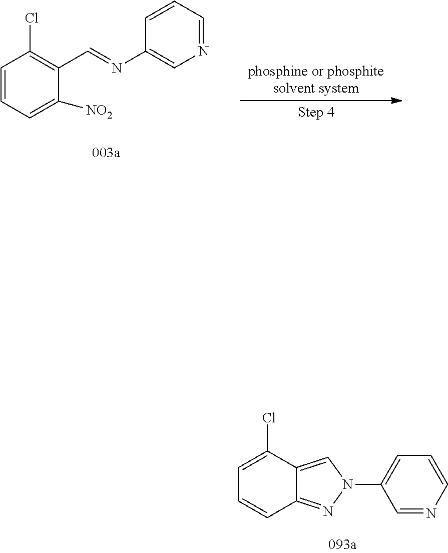 [SMILES: O=[N+]([O-])c1cccc(Cl)c1/C=N/c1cccnc1 ; Clc1cccc2nn(-c3cccnc3)cc12]
wherein the phosphine is selected from tri (C₁₋₄) alkyl phosphine and triaryl phosphine and the phosphite is selected from tri (C₁₋₄) alkyl phosphite and triaryl phosphite.

2. The process of claim 1, wherein for step 1:
(1) the source of Br is (a) selected from N-bromosuccinimide, Br₂, HBr and H₂O₂ or a combination thereof, or (b) N-bromosuccinimide;
(2) (a) the reaction is run under photochemical conditions. (b) the reaction is promoted by a light source, (b) the reaction is promoted by visible light, (c) the reaction is promoted by visible light provided by about 400W of a light source; and/or
(3) (a) A photosensitive or thermally activated radical initiator is used or (b) the radical initiator is azobisisobutyronitrile;
(4) (a) the solvent system comprises at least one polar aprotic solvent (b) the solvent system comprises dichloromethane, (c) the solvent system comprises a combination of least one polar aprotic solvent and at least one polar protic solvent, or (d) the solvent system comprises dichloromethane and water.

3. The process of claim 1 or claim 2, wherein for step 2(a):
(1) the weak base (a) is an inorganic base, (b) is a carbonate or a bicarbonate, (c) is selected from sodium carbonate, sodium bicarbonate, potassium carbonate, potassium bicarbonate, and magnesium carbonate, or (d) sodium carbonate; and/or
(2) (a) the solvent system comprises at least one polar solvent, (b) the solvent system comprises water, (c) the solvent system comprises acetonitrile or (d) the solvent system comprises water and acetonitrile.

4. The process of claim 1 or claim 3, wherein for step 2(b):
(1) the oxidizing reagent is NaOCl;
(2) the phase transfer catalyst is (a) a quaternary ammonium salt or (b) tetrabutylammonium bromide; and/or
(3) the solvent system (a) comprises a non-polar solvent or (b) comprises toluene.

5. The process of any one of claims 1 to 4, wherein for step 2:
(1) the oxidizing reagent is (a) a N-oxide reagent or (b) trimethylamine N-oxide; and/or
(2) the solvent system (a) comprises or predominately comprises at least one polar solvent or (b) comprises DMF.

6. The process of any one of claims 1 to 5, wherein for step 3:
(1) the solvent system (a) comprises a non-polar solvent or (b) comprises toluene; and/or
(2) the reaction is run at reflux with concomitant azeotropic distillation of water.

7. The process of any one of claims 1 to 6, wherein for step 4:
(1) the solvent system (a) comprises or predominately comprises a polar protic solvent, (b) comprises or predominately comprises a C₁₋₄ alcohol, (c) comprises or predominately comprises isopropyl alcohol, (d) comprises or predominately comprises a non-polar solvent, or (e) comprises or predominately comprises toluene;
(2) the phosphine or phosphite is triethyl phosphite; and/or
(3) the reaction is run at reflux.

8. The process of any one of claims 1 to 7, wherein any of compounds 181a, 050, 378, 003a, and 093a are optionally isolated from the reaction product mixture.

9. The process of any one of claims 1 to 7, wherein any two sequential reactions, or all of the sequential reactions from compound 339 to compound 093a are done in a one pot scheme.

10. A process for the preparation of compound 093a and 093b according to the following scheme
(1) Step 1 comprising forming a reaction mixture comprising compound 114a or 114b, an oxidizing agent, and a solvent system, and reacting the reaction mixture to form a reaction product mixture comprising compound 115a or 115b according to the following reaction scheme 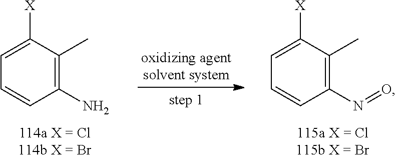
(2) Step 2 comprising forming a reaction mixture comprising compound 115a or 115b, a source of bromine or a source of chlorine, and a solvent system, and reacting the reaction mixture under photochemical conditions to form a reaction product mixture comprising any one of compounds 116a-d according to the following reaction scheme 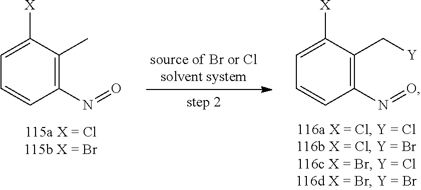
and
(3) Step 3 comprising forming a reaction mixture comprising compound any one of compounds 116a-d, compound 520, and a solvent system, and reacting the reaction mixture to form a reaction product mixture comprising compound 093a or 093b according to the following reaction scheme

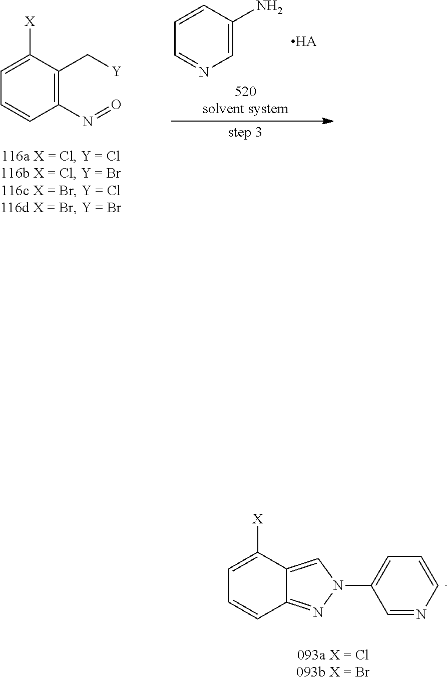

11. The process of claim 10, wherein for step 1 of the first scheme for preparing compound 093a or 093b:
(1) the oxidizing agent is (a) a peroxide containing reagent, (b) selected from potassium peroxymonosulfate, sodium perborate, sodium percarbonate, hydrogen peroxide, peracetic acid, or 3-chloroperbenzoic acid, or (c) potassium peroxymonosulfate; and/or
(2) (a) the solvent system comprises at least one polar aprotic solvent and at least one polar protic solvent or (b) the solvent system comprises dichloromethane and water or the solvent system comprises dichloroethane and water.

12. The process of claim 10 or 11, wherein for step 2 for preparing compound 093a or 093b:
(1) (a) the source of Br is N-bromosuccinimide, Br₂, or HBr and hydrogen peroxide, (b) the source of Br is Br₂, (c) the source of Cl is N-chlorosuccinimide, trichloroisocyanuric acid, Cl₂, or HCl and hydrogen peroxide, or (d) the source of Cl is Cl₂; and/or
(2) (a) the process comprises a light source;
(3) (a) A photosensitive or thermally activated radical initiator is used or (b) the radical initiator is azobisisobutyronitrile;
(4) (a) the solvent system comprises at least one polar aprotic solvent and at least one polar protic solvent or (b) the solvent system comprises dichloromethane and water or dichloroethane and water.

13. The process of any one of claims 10 to 12, wherein for step 3 for preparing compound 093a or 093b:
(1) Compound 520 is (a) a hydrochloride, or hydrobromide, sulfate, methanesulfonate, or p-toluenesulfonate salt, (b) a hydrochloride salt, (c) prepared in situ, or (d) prepared and isolated prior to use;
(2) The solvent system comprises at least one polar aprotic solvent and at least one polar protic solvent, or (b) comprises dichloromethane and water, dichloroethane and water, or toluene and water.

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. A process for the preparation of compound 093a or 93b according to the following scheme which comprises two steps:
(1) Step 1 comprising forming a reaction mixture comprising compound 181a or 181b, compound 520 (3-aminopyridinium salt), and a solvent system, and reacting the reaction mixture to form a reaction product mixture comprising compound 182a or 182b according to the following reaction scheme first scheme for preparing compound 093a or 93b. 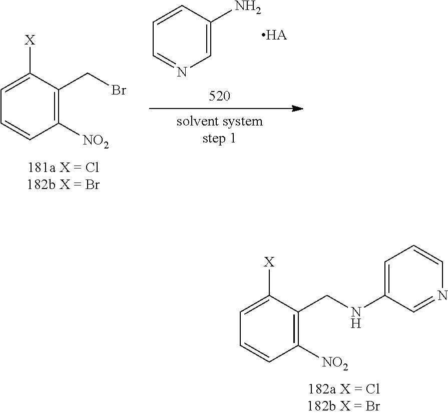
(2) Step 2 comprises forming a reaction mixture comprising compound 182a or 182b, a reductant, a base, and a solvent system, and reacting the reaction mixture to form a reaction product mixture comprising compound 093a or 93b according to the following reaction scheme

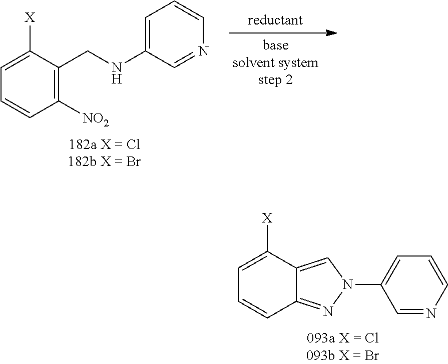

26. The process of claim 25, wherein for step 1 of the scheme for preparing compound 093a or 93b
(1) Compound 520 is (a) a inorganic or organic acid salt, (b) a hydrochloride, hydrobromide, sulfate, formic, methanesulfonate, or p-toluenesulfonate salt, (c) the methanesulfonate salt, (d) prepared in situ, or (e) prepared and isolated before use;
(2) The solvent system (a) comprises a polar aprotic solvent, (b) is selected from acetonitrile, benzonitrile, sulfolane, (c) comprises a combination of a non-polar solvent and a polar protic solvent, (d) is a combination of toluene and water.
(3) When the solvent system comprises toluene and water (a) a phase transfer catalyst is also included, (b) the phase transfer catalyst included is a quaternary ammonium salt, (c) the phase transfer catalyst included is tetrabutylammonium bromide.
(4) Compound 182a or 182b is (a) isolated as a salt, (b) isolated as the anilinium bromide salt, the di-bromide salt, or a salt mixture of HBr and methanesulfonic acid or p-toluenesulfonic acid, or (c) is treated with a base after the reaction and is isolated as the free base.

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. A polymorph of N-(1-methylcyclopropyl)-2-(3-pyridinyl)-2H-indazole 4-carboxamide designated Form A characterized by a X-ray powder diffraction pattern having at least 20 reflections positions
2θ
7.9
13.6
15.5
17.4
20.4
21.7
22.4
27.2
28.2
34.0

39. A polymorph of N-(1-methylcyclopropyl)-2-(3-pyridinyl)-2H-indazole 4-carboxamide designated Form B characterized by a X-ray powder diffraction pattern having at least 20 reflections positions
2θ
8.6
13.1
13.9
16.0
17.2
20.5
20.9
22.4
26.0
29.1
